(19) United States
(12) Patent Application Publication
(10) Pub. No.: US 2025/0177765 A1
(43) Pub. Date: Jun. 5, 2025

(54) TUMOR TREATING FIELDS SYSTEM AND INSULATED ELECTRODE THEREOF

(71) Applicant: JIANGSU HEALTHY LIFE INNOVATION MEDICAL TECHNOLOGY CO., LTD, Jiangsu (CN)

(72) Inventors: Jun ZHANG, Jiangsu (CN); Hu SUN, Jiangsu (CN); Jing YU, Jiangsu (CN); Sheng CHEN, Jiangsu (CN); Jianjun YING, Jiangsu (CN); Qichao SHEN, Jiangsu (CN); Yidong SUN, Jiangsu (CN); Jiajie HUI, Jiangsu (CN)

(21) Appl. No.: 18/723,209

(22) PCT Filed: Dec. 20, 2022
(86) PCT No.: PCT/CN2022/140249

§ 371 Date: Feb. 18, 2025

(30) Foreign Application Priority Data
Dec. 22, 2021 (CN) 202111578573.9
Dec. 22, 2021 (CN) 202111578597.4
Dec. 22, 2021 (CN) 202111580105.5
Dec. 22, 2021 (CN) 202111580121.4
Dec. 22, 2021 (CN) 202111580125.2
Dec. 22, 2021 (CN) 202111580130.3
Dec. 22, 2021 (CN) 202111580142.6
Dec. 22, 2021 (CN) 202111580208.1
Dec. 24, 2021 (CN) 202111596993.X
Dec. 24, 2021 (CN) 202111601004.1

(51) Int. Cl. A61N 1/40

(52) U.S. Cl. CPC A61N 1/40

(57) ABSTRACT

A tumor treating fields system and an associated insulated electrode includes at least one electrode sheet applying an alternating electric signal; and an electrical connector detachably connected with the sheet, the sheet includes an individual electrode unit and a first wire connected with the electrode unit, the sheet is detachably connected with the electrical connector through the first wire. The sheet of the insulated electrode is detachably connected with the electrical connector through the first wire, and the sheet includes one electrode unit. When the electrode unit is damaged and is nonfunctional, only the damaged sheet is replaced, reducing the cost of tumor treatment. The electrode units are combined in quantity or adjusted in position according to a patient's tumor site, position and tumor size, to ensure the coverage area for tumor treating fields therapy performed by the insulated electrodes, enhancing the electric field strength for tumor treating fields therapy.

### CROSS-REFERENCE TO RELATED APPLICATION(S)

The present application is a us national application of PCT application numbered as PCT/CN/2022/140249 which claims priority to Chinese Patent Application No. 202111580142.6 field on Dec. 22, 2021, Chinese Patent Application No. 202111596993.X field on Dec. 24, 2021, Chinese Patent Application No. 202111601004.1 field on Dec. 24, 2021, Chinese Patent Application No. 202111580208.1 field on Dec. 22, 2021, Chinese Patent Application No. 202111578597.4 field on Dec. 22, 2021, Chinese Patent Application No. 202111580105.5 field on Dec. 22, 2021, Chinese Patent Application No. 202111580121.4 field on Dec. 22, 2021, Chinese Patent Application No. 202111580130.3 field on Dec. 22, 2021, Chinese Patent Application No. 202111578573.9 field on Dec. 22, 2021, and Chinese Patent Application No. 202111580125.2 field on Dec. 22, 2021, the entire disclosures of which are incorporated herein by reference.

### FIELD

The present application relates to a tumor treating fields system and an insulated electrode thereof, which belongs to the technical field of medical devices.

### BACKGROUND

At present, methods of treating tumor mainly include surgery, radiotherapy, chemotherapy, etc. However, each of them has corresponding shortcomings. For example, radiotherapy and chemotherapy may produce side effects and kill normal cells. Using electric fields to treat tumor is also one of the frontiers of current research. Tumor treating fields therapy is a tumor treatment method that produces alternating electric fields with low-intensity and medium-high frequency by a special electric field generator to interfere the mitosis process of tumor cells. Studies have shown that, the tumor treating fields therapy is significantly effective in the treatment of glioblastoma, non-small cell lung cancer, malignant pleural mesothelioma and other diseases. The electric fields applied by such treatment method may affect the aggregation of tubulin, prevent the formation of spindle, inhibit the mitosis process and induce the apoptosis of cancer cells.

In general, the existing tumor treating fields system includes an electric field generator and two pairs of insulated electrodes electrically connected with the electric field generator and attached to the body surface corresponding to the patient's tumor site. The electric field generator generates the alternating electrical signals needed for the tumor treating fields therapy, and applies the alternating electrical signals to the two pairs of insulated electrodes electrically connected thereto, so as to further apply the alternating electric field to the patient through the insulated electrodes attached to the body surface corresponding to the patient's tumor site for the tumor treating fields therapy. Each of the existing insulated electrodes includes a flexible circuit board, a plurality of ceramic sheets arranged on the flexible circuit board, and a wire electrically connected with the flexible circuit board, as disclosed by the China Patent Publication No. 112717272 or the China Patent Publication No. 113164745. The flexible circuit board includes a flexible substrate, a plurality of conductive traces embedded in the flexible substrate, and a plurality of conductive pads exposed from the flexible substrate and electrically connected with a same conductive trace. The plurality of ceramic sheets are arranged on the flexible circuit board by welding with the corresponding conductive pads, and then are connected in series through a conductive trace electrically connected with all the conductive pads. One end of the wire is electrically connected with the flexible circuit board, and the other end of the wire is provided with a plug which may be plugged with the electric field generator.

The above insulated electrode of the tumor treating fields system is plugged with the electric field generator through the plug arranged at one end of the wire to realize the electrical connection between the insulated electrode and the electric field generator, and then the alternating current signals generated by the electric field generator are transmitted to the flexible circuit board through the wire, and then the alternating current signals are transmitted to all conductive pads through the conductive trace electrically connected with all the conductive pads and the flexible circuit board. Further, the alternating current signals are simultaneously transmitted to all ceramic sheets connected in series through that conductive trace connected with all the conductive pads, so that the alternating electric field may be applied to the patient's tumor site through the plurality of ceramic sheets arranged on the flexible circuit board for tumor treating fields therapy. Although the tumor treating fields system can apply the alternating electric signals to the patient's tumor site through the plurality of ceramic sheets arranged on the flexible circuit board, the ceramic sheets are all connected in series through the same conductive trace of the flexible circuit board, and thus there is a problem that the alternating electric signal cannot be transmitted to the ceramic sheets due to the fracture of the conductive trace of the flexible circuit board or the poor welding of a certain ceramic sheet, which may result in the insulated electrode being totally scrapped and cannot be used due to being tested as unqualified during manufacturing. It then results in low product manufacturing yield and increased manufacturing cost, and further affects the treatment effects due to the insufficient alternating electric field strength applied to the patient's tumor site when in use. In addition, since all the conductive pads on the flexible circuit board are connected in series through a conductive trace, it is necessary to conduct electrical tests on the flexible circuit board before the ceramic sheets are welded to the flexible circuit board. After the ceramic sheets are welded to the flexible circuit board, it needs to again conduct electrical tests to confirm them one by one, resulting in complicated working procedures and low efficiency.

In addition, since the ceramic sheets of the insulated electrode used for applying alternating electric signals to the patient's tumor site in the above tumor treating fields system are all welded to the flexible circuit board through the conductive pads on the flexible circuit board, the relative positions of the plurality of ceramic sheets are fixed and unchangeable. The number of the ceramic sheets is also fixed, and cannot be freely increased or decreased. However, the positions, locations, and sizes of patients' tumors are different from each other. If the above insulated electrode is always used for treatment, it may result in insufficient electric field strength applied to the tumor site for treatment due to the insufficient number of ceramic sheets for applying alternating electric signals or the improper position of some ceramic sheets, or the electric field does not cover part of the tumor area, thus affecting the treatment effect.

Therefore, it is necessary to provide an improved insulated electrode and a tumor treating fields system to overcome the problems existing in the prior art.

### SUMMARY

The present application provides an insulated electrode and a tumor treating fields system that may easily replace the electrode sheet when the electrode sheet is damaged, and the electrode sheets may be freely combined to ensure the treating effect.

The insulated electrode of the present application may be implemented by the following technical solution: An insulated electrode for tumor treating fields therapy, comprises: at least one electrode sheet configured to apply an alternating electric signal; and an electrical connector detachably connected with the electrode sheet, wherein the electrode sheet comprises: an individual electrode unit; and a first wire electrically connected with the electrode unit, wherein the electrode sheet is detachably connected with the electrical connector through the first wire.

Preferably, a plurality of electrode sheets are connected to the electrical connector in parallel through the corresponding first wires.

Preferably, the first wire of the electrode sheet has a first plug detachably plugged with the electrical connector, wherein the first plug and the electrode unit are located at opposite ends of the first wire respectively.

Preferably, the electrical connector has a plurality of sockets which can be detachably plugged with the first plugs of the first wires of the corresponding electrode sheets.

Preferably, the electrical connector is further provided with a second wire, wherein the second wire and the plurality of sockets are respectively located at opposite ends of the electrical connector.

Preferably, the second wire has a second plug arranged at an end thereof.

Preferably, the electrical connector has a body, wherein the plurality of sockets and the second wire are respectively arranged at opposite ends of the body.

Preferably, the electrode sheet further comprises a wiring part connected with the electrode unit, wherein the wiring part is welded with an end of the first wire away from the first plug.

Preferably, the electrode unit comprises a main body part and a dielectric element welded on one side of the main body part, wherein the wiring part is laterally extended from the main body part.

Preferably, the wiring part and the main body part of the electrode unit form a flexible circuit board of the electrode sheet.

Preferably, the electrode unit further comprises at least one temperature sensor, wherein the temperature sensor is arranged on the main body part and at the same side as the dielectric element.

Preferably, the dielectric element is configured with at least one perforation extended through the middle thereof, wherein the temperature sensor is accommodated in the corresponding perforation of the dielectric element.

Preferably, the electrode unit further comprises an insulating plate adhered to a side of the main body part away from the dielectric element.

Preferably, the periphery of a welding point of the first wire and the wiring part is covered with a heat shrinkable sleeve.

Preferably, the first wire is detachably connected with the electrode unit.

Preferably, the electrode sheet comprises a wiring part electrically connected with the electrode unit, wherein an end of the wiring part away from the electrode unit is configured with a docking socket.

Preferably, an end of the first wire away from the first plug is provided with a docking plug, wherein the docking plug is detachably plugged with the docking socket.

Preferably, the electrode sheet further comprises a backing adhered to the electrode unit: a support member arranged around the electrode unit and adhered to the backing and an adhesive covering sides of the electrode unit and the support member away from the backing.

The tumor treating fields system of the present application may be implemented by the following technical solution: A tumor treating fields system comprises an electric field generator; and the above insulated electrode connected with the electric field generator.

Preferably, the system also comprises an adapter electrically connected with the electric field generator, wherein the insulated electrode is detachably assembled on the adapter and is electrically connected with the electric field generator through the adapter.

Preferably, the insulated electrode is detachably assembled on the electric field generator.

The electrode sheets of the insulated electrode of the present application are detachably connected with the electrical connector through the first wires thereof, and each electrode sheet only comprises one electrode unit, so that when the electrode unit is damaged and is unable to work, it may replace the electrode sheet containing the damaged electrode unit without scrapping all the electrode sheets, which may reduce the scrap cost. In addition, the electrode sheets of the insulated electrodes of the present application may be freely combined in quantity or freely adjusted in position according to a patient's tumor site, tumor position and tumor size, so as to ensure the coverage area for tumor treating fields therapy performed by the insulated electrodes, ensure the electric field strength for tumor treating fields therapy performed by the insulated electrodes, and ensure the effect of the treating fields therapy. At the same time, the adjustment of the relative positions between the plurality of electrode sheets allow the skin of the body surface of a patient's site where the electrode sheets are adhered to breathe freely, so as to avoid accumulation of heat on the body surface corresponding to the patient's tumor site caused by a long-term treating fields therapy, which causes sweating and clogging pores and results in skin inflammation.

The insulated electrode of the present application may be implemented by the following technical solution. An insulated electrode for tumor treating fields therapy comprises: a flexible circuit board: a dielectric element and a temperature sensor both arranged on a same side of the flexible circuit board; and a wire electrically connected with the flexible circuit board. The temperature sensor has a ground terminal and a signal terminal. The flexible circuit board has an insulating substrate and three conductive traces embedded in the insulating substrate; wherein one conductive trace of the three conductive traces is electrically connected with the dielectric element, one conductive trace is electrically connected with the ground terminal of the temperature sensor, and one conductive trace is electrically connected with the signal terminal of the temperature sensor. The wire is electrically connected with the three conductive traces of the flexible circuit board.

Preferably, the flexible circuit board has three golden fingers exposed from the insulating substrate and electrically connected with the corresponding parts of the wire.

Preferably, the three golden fingers are respectively electrically connected with three conductive traces of the flexible circuit board.

Preferably, the flexible circuit board is configured with a conductive pad corresponding to the dielectric element, wherein the conductive pad is welded with the dielectric element.

Preferably, the conductive pad is exposed from the insulating substrate and connected to a conductive trace of the flexible circuit board that electrically connected with the dielectric element.

Preferably, the conductive pad comprises a plurality of conductive cores arranged at intervals, wherein the plurality of conductive cores are connected in series by the conductive trace of the flexible circuit board that electrically connected with the dielectric element.

Preferably, the flexible circuit board is configured with a pair of pads exposing the insulating substrate and corresponding to the temperature sensor.

Preferably, one of the two pads is welded with the ground terminal of the temperature sensor, and the other pad of the two pads is welded with the signal terminal of the temperature sensor.

Preferably, one of the two pads is connected to the conductive trace of the flexible circuit board that electrically connected with the ground terminal of the temperature sensor, and the other pad is connected to the conductive trace of the flexible circuit board that electrically connected with the signal terminal of the temperature sensor.

Preferably, one end of the wire is electrically connected with the flexible circuit board, and the other end of the wire is provided with a plug.

Preferably, a heat shrinkable sleeve is arranged at a joint of the wire and the flexible circuit board.

Preferably, the dielectric element is configured with a perforation extended therethrough, wherein the temperature sensor is accommodated in the perforation.

Preferably, among the three conductive traces, the conductive trace electrically connected with the dielectric element is a first conductive trace, the conductive trace electrically connected with the ground terminal of the temperature sensor is the second conductive trace, and the conductive trace electrically connected with the signal terminal of the temperature sensor is the third conductive trace. The flexible circuit board is configured with a conductive pad that connected with the first conductive trace. The flexible circuit board is configured with two pads, wherein one pad is connected with the second conductive trace and the other pad is connected with the third conductive trace.

Preferably, the conductive pad and the pads are arranged on the same side of the flexible circuit board.

Preferably, both the conductive pad and the two pads are exposed from the insulating substrate of the flexible circuit board.

Preferably; the flexible circuit board further has three golden fingers welded with the wire, wherein the golden fingers are exposed from the insulating substrate of the flexible circuit board.

Preferably, the golden fingers, the conductive pad and the two pads are located at the same side of the flexible circuit board.

Preferably, the insulated electrode further comprises a backing adhered to a corresponding part of the flexible circuit board.

Preferably, the insulated electrode further comprises an insulating plate arranged at a side of the flexible circuit board far away from the dielectric element, wherein the insulating plate corresponds to the dielectric element along the thickness direction. The insulating plate is sandwiched between the flexible circuit board and the backing.

The insulated electrode of the present application may be implemented by the following technical solution: An insulated electrode for tumor treating fields therapy comprises: a flexible circuit board, a single dielectric element electrically connected with the flexible circuit board; and a plurality of temperature sensors. The number of the temperature sensors is n, wherein n is an integer greater than 1 and not greater than 8. The temperature sensor has a ground terminal and a signal terminal. The flexible circuit board has an insulating substrate and a plurality of conductive traces embedded in the insulating substrate, wherein the plurality of conductive traces are n+2 conductive traces. Among the conductive traces, one conductive traces is electrically connected with the dielectric element, one conductive trace is electrically connected with ground terminals of all the temperature sensors, and the rest conductive traces are electrically connected with corresponding signal terminals of the temperature sensors respectively.

Preferably, the flexible circuit board has a wiring part electrically connected with both the dielectric element and the temperature sensor, wherein both the dielectric element and the temperature sensor are located at one end of the wiring part.

Preferably, the insulated electrode further comprises a wire electrically connected with the wiring part of the flexible circuit board. The wire and the dielectric element are located at opposite ends of the wiring part respectively.

Preferably; one end of the wire is electrically connected with the wiring part of the flexible circuit board, and the other end of the wire is provided with the plug.

Preferably, the flexible circuit board is configured with a conductive pad welded with the dielectric element, wherein the conductive pad is arranged at one end of the wiring part.

Preferably, the conductive pad is exposed from the insulating substrate and connected to the conductive trace of the flexible circuit board that electrically connected with the dielectric element.

Preferably, the n temperature sensors are all arranged in an area surrounded by conductive pad, wherein an extension direction of a straight line where the n temperature sensors are located is consistent with an extension direction of the wiring part.

Preferably, the conductive pad comprises a plurality of conductive cores arranged at intervals, wherein the plurality of conductive cores are connected in series by the conductive trace of the flexible circuit board that is electrically connected with the dielectric element.

Preferably, the plurality of conductive cores are arranged in a matrix at intervals, wherein, among the plurality of conductive cores, four conductive cores located in adjacent rows and adjacent columns are center-symmetrically arranged.

Preferably, the n temperature sensors are respectively arranged offset from the symmetrical center of the four conductive cores corresponding to the conductive pad.

Preferably, there are two temperature sensors, wherein one of the two temperature sensors is arranged on a side of the symmetrical center of the corresponding four conductive cores away from the wiring part, and the other one of the two temperature sensors is arranged on a side of the symmetrical center of the corresponding four conductive cores close to the wiring part.

Preferably, the flexible circuit board is configured with n pairs of pads corresponding to the temperature sensors and located at one end of the wiring part, wherein the n pairs of pads and the conductive pad are located at the same end of the wiring part.

Preferably, each pair of pads comprises a first pad and a second pad, wherein the first pad is welded with the ground terminal of the corresponding temperature sensor, and the second pad is welded with the signal terminal of the corresponding temperature sensor.

Preferably, each pair of pads is arranged offset from the symmetrical center of the corresponding four conductive cores.

Preferably, there are two pairs of pads, wherein one pair of pads is arranged on a side of the symmetrical center of the corresponding four conductive cores away from the wiring part, and the other pair of pads is arranged on a side of the symmetrical center of the corresponding four conductive cores close to the wiring part.

Preferably, a straight line where the symmetry center of each pair of the n pairs of pads is located is parallel to an extension direction of the wiring part.

Preferably, the first pad is connected to the conductive trace of the flexible circuit board that is electrically connected with the ground terminal of the temperature sensor, and the second pads are respectively connected to the corresponding conductive traces of the flexible circuit board that are electrically connected with the signal terminals of the corresponding temperature sensors.

Preferably, the dielectric element has a perforation arranged corresponding to the temperature sensor, wherein the temperature sensor is accommodated in the corresponding perforation.

Preferably, the number of the temperature sensors is two, the number of the conductive traces is four, and the number of the conductive cores is six.

Preferably, the insulated electrode further comprises a backing adhered to a corresponding part of the flexible circuit board.

Preferably, the insulated electrode further comprises an insulating plate arranged opposite to the dielectric element, wherein the insulating plate is arranged corresponding to the dielectric element along the thickness direction. The insulating plate is sandwiched between the dielectric element and the backing.

The insulated electrode of the present application may be implemented by the following technical solution: An insulated electrode for tumor treating fields therapy comprises: a flexible circuit board: a dielectric element and a plurality of temperature sensors arranged at the same side of the flexible circuit board; and a wire electrically connected with the flexible circuit board. The number of the temperature sensors is n, wherein n is an integer greater than 1 and not greater than 8. Each temperature sensor has a ground terminal and a signal terminal. The flexible circuit board has an insulating substrate and a plurality of conductive traces embedded in the insulating substrate, wherein the plurality of conductive traces are n+2 conductive traces. One of the conductive traces is electrically connected with the dielectric element, one of the conductive traces is electrically connected with ground terminals of all the temperature sensors, and the rest conductive traces are electrically connected with corresponding signal terminals of the temperature sensors respectively. The wire is electrically connected with the plurality of conductive traces of the flexible circuit board.

Preferably, the flexible circuit board has a plurality of golden fingers exposing the insulating substrate and electrically connected with the corresponding parts of the wire.

Preferably, the golden fingers are electrically connected with the conductive traces of the flexible circuit board respectively.

Preferably, the number of the temperature sensors is two, the number of the conductive traces is four, and the number of the golden fingers is four.

Preferably, the flexible circuit board is configured with a conductive pad corresponding to the dielectric element, wherein the conductive pad is welded with the dielectric element.

Preferably, the conductive pad is exposed from the insulating substrate and connected to the conductive trace of the flexible circuit board that electrically connected with the dielectric element.

Preferably, the conductive pad comprises a plurality of conductive cores arranged at intervals, wherein the plurality of conductive cores are connected in series by the conductive trace of the flexible circuit board that electrically connected with the dielectric element.

Preferably, the flexible circuit board is configured with n pairs of pads, wherein each pair of pads is located between two corresponding conductive cores arranged at intervals.

Preferably, each pair of pads is arranged on the flexible circuit board at a position corresponding to the corresponding temperature sensor, wherein each pair of pads is exposed from the insulating substrate of the flexible circuit board.

Preferably, each pair of pads comprises a first pad and a second pad, wherein the first pad is welded with the ground terminal of the corresponding temperature sensor, and the second pad is welded with the signal terminal of the corresponding temperature sensor.

Preferably, the first pad is connected to the conductive trace of the flexible circuit board that electrically connected with the ground terminal of the temperature sensor, and each second pad is respectively connected to the conductive trace of the flexible circuit board that electrically connected with the signal terminal of the corresponding temperature sensor.

Preferably, one end of the wire is electrically connected with the flexible circuit board, and the other end of the wire is provided with a plug.

Preferably, a heat shrinkable sleeve is arranged at a joint of the wire and the flexible circuit board.

Preferably, the dielectric element has a plurality of perforations arranged corresponding to the temperature sensors, wherein the temperature sensor is accommodated in the corresponding perforation.

Preferably, the conductive trace electrically connected with the dielectric element is the first conductive trace, the conductive trace that electrically connected with the ground terminals of the temperature sensors is the second conductive trace, and the rest n conductive traces that respectively and electrically connected with signal terminals of the corresponding temperature sensors are third conductive traces. The flexible circuit board is configured with a conductive pad that connected with the first conductive trace. The flexible circuit board is configured with n pairs of pads, wherein one pad of each pair of pads is connected with the second conductive trace and the other pad of each pair of pads is connected with the corresponding third conductive trace.

Preferably, the conductive pad and the pads are arranged on the same side of the flexible circuit board.

Preferably, both the conductive pad and the pads are exposed from the insulating substrate of the flexible circuit board.

Preferably, the flexible circuit board further has a plurality of golden fingers welded with the wire, wherein the golden fingers are all exposed from the insulating substrate of the flexible circuit board. There are n+2 golden fingers, wherein n is an integer greater than 1 and not greater than 8.

Preferably, there are four golden fingers, two temperature sensors, two pairs of pads, and two third conductive traces.

Preferably; the golden fingers, the conductive pad and the two pairs of pads are located at the same side of the flexible circuit board.

Preferably, the insulated electrode further comprises a backing adhered to a corresponding part of the flexible circuit board.

Preferably, the insulated electrode further comprises an insulating plate arranged at a side of the flexible circuit board far away from the dielectric element, wherein the insulating plate corresponds to the dielectric element along the thickness direction. The insulating plate is sandwiched between the flexible circuit board and the backing.

The insulated electrode of the present application may be implemented by the following technical solution. An insulated electrode for tumor treating fields therapy comprises: a flexible circuit board: at least one dielectric element arranged on the flexible circuit board; and a wire electrically connected with the flexible circuit board. The flexible circuit board is provided with a wiring part welded with the wire and a reinforcing plate arranged on the wiring part, wherein the wiring part is provided with a plurality of golden fingers welded with the wire and conductive traces electrically connected with the corresponding golden fingers respectively. The reinforcing plate is arranged at a position opposite to the connection position of the conductive traces and the golden fingers of the wiring part.

Preferably, the reinforcing plate and the dielectric element are located on opposite sides of the flexible circuit board.

Preferably, the plurality of golden fingers are arranged on a side of the wiring part that is the same as the dielectric element.

Preferably, the reinforcing plate is made of polyimide material with a thickness of 0.6 mm-1 mm.

Preferably, the reinforcing plate is made of epoxy glass fiber material with a thickness of 0.2 mm-0.5 mm.

The tumor treating fields system of the present application may be implemented by the following technical solution. A tumor treating fields system comprises: a first pair of the above-mentioned insulated electrodes configured on the surface of a patient's head: a second pair of the above-mentioned insulated electrodes configured on the surface of the patient's head: a control signal generator, which generates a periodic control signal with a first output state and a second output state, wherein the first output state has a first time period t1, the second output state has a second time period t2, the first time period t1 and the second time period t2 are both between 400 ms and 980 ms: an AC signal generator, which generates a first AC signal with a first frequency between the first pair of insulated electrodes when the control signal is in the first output state and a second AC signal with a second frequency between the second pair of insulated electrodes when the control signal is in the second output state, wherein the first frequency is the same as the second frequency, and wherein the switching between generation of the first AC signal between the first pair of insulated electrodes and the second AC signal between the second pair of insulated electrodes by the AC signal generator are implemented by controlling the control signal generator to switch between the first output state and the second output state.

Preferably, the first time period t1 and the second time period t2 have the same duration.

Preferably, the first time period t1 and the second time period t2 are both 50% of a work cycle.

Preferably, the first AC signal has an amplitude that rises in the switch-to-turn-on period t3 and falls in the switch-to-turn-off period t4 in each of the first time periods t1, and the second AC signal has an amplitude that rises in the switch-to-turn-on period t3 and falls in the switch-to-turn-off period t4 in each of the second time periods t2.

Preferably, the duration of the switch-to-turn-on period t3 and the switch-to-turn-off period t4 are both less than 10% of the duration of the first or second time period t1, t2.

Preferably, the first AC signal is applied to the first pair of insulated electrodes to generate a first electric field between the first pair of insulated electrodes, and the second AC signal is applied to the second pair of insulated electrodes to generate a second electric field between the second pair of insulated electrodes.

Preferably, the direction of the first electric field is perpendicular to the direction of the second electric field.

Preferably, the periodic control signal is a periodic square wave signal.

Preferably, both the first AC signal and the second AC signal have a field strength of at least 1V/cm.

Preferably, the system further includes an inverter: a first switch/amplifier module; and a second switch/amplifier module. A control terminal of the first switch/amplifier module is directly connected with the control signal generator, and a control terminal of the second switch/amplifier module is connected with the control signal generator through the inverter. Input terminals of the first switch/amplifier module and the second switch/amplifier module are both connected with the AC signal generator. An output terminal of the first switch/amplifier module is connected with the first pair of insulated electrodes, and an output terminal of the second switch/amplifier module is connected with the second pair of insulated electrodes.

### BRIEF DESCRIPTION OF THE DRAWINGS

FIG. 1 illustrates a block diagram of an embodiment of a tumor treating fields system of the present application.

FIG. 2 is a schematic diagram of control signals for turning on or off a first electric field and a second electric field in the tumor treating fields system shown in FIG. 1.

FIG. 3 shows a relationship between cell growth rate and electric field work cycle.

FIG. 4 is a schematic diagram of an AC signal applied to one pair of the insulated electrodes shown in FIG. 1.

FIG. 5 is a three-dimensional assembled view of a first embodiment of an insulated electrode of the tumor treating fields system shown in FIG. 1.

FIG. 6 is a three-dimensional exploded view of the insulated electrode shown in FIG. 4.

FIG. 7 is a three-dimensional exploded view of a wire and an electrical functional component of the insulated electrode shown in FIG. 6.

FIG. 8 is a front wiring diagram of a flexible circuit board of the electrical functional component shown in FIG. 7.

FIG. 9 is a back wiring diagram of a flexible circuit board of the electrical functional component shown in FIG. 7.

FIG. 10 a three-dimensional assembled view of a second embodiment of an insulated electrode of the tumor treating fields system shown in FIG. 1.

FIG. 11 a three-dimensional exploded view of the insulated electrode shown in FIG. 10.

FIG. 12 is a three-dimensional exploded view of a wire and an electrical functional component of the insulated electrode shown in FIG. 11.

FIG. 13 is a planar diagram of a flexible circuit board of the electrical functional component shown in FIG. 12.

FIG. 14 is a front wiring diagram of the flexible circuit board of the electrical functional component shown in FIG. 13.

FIG. 15 is a back wiring diagram of the flexible circuit board of the electrical functional component shown in FIG. 13.

FIG. 16 is a three-dimensional view of an alternate embodiment of the insulated electrode of the second embodiment shown in FIG. 10.

FIG. 17 is a three-dimensional assembled view of a third embodiment of an insulated electrode of the tumor treating fields system shown in FIG. 1.

FIG. 18 is a three-dimensional exploded view of the insulated electrode shown in FIG. 17.

FIG. 19 is a three-dimensional exploded view of a wire and an electrical functional component of the insulated electrode shown in FIG. 18.

FIG. 20 is a planar diagram of a flexible circuit board of the electrical functional component shown in FIG. 19.

FIG. 21 is a front wiring diagram of the flexible circuit board shown in FIG. 20.

FIG. 22 is a back wiring diagram of the flexible circuit board shown in FIG. 20.

FIG. 23 is a three-dimensional exploded view of an alternate embodiment of the insulated electrode of the third embodiment shown in FIG. 17.

FIG. 24 is an assembled view of a fourth embodiment of an insulated electrode of the tumor treating fields system shown in FIG. 1.

FIG. 25 is an exploded view of an electrical connector and an electrode sheet of the insulated electrode shown in FIG. 24.

FIG. 26 is a three-dimensional exploded view of the electrode sheet shown in FIG. 25.

FIG. 27 is a three-dimensional exploded view of a first wire and an electrical functional component of the electrode sheet shown in FIG. 26.

FIG. 28 is a planar diagram of a flexible circuit board of the electrode sheet shown in

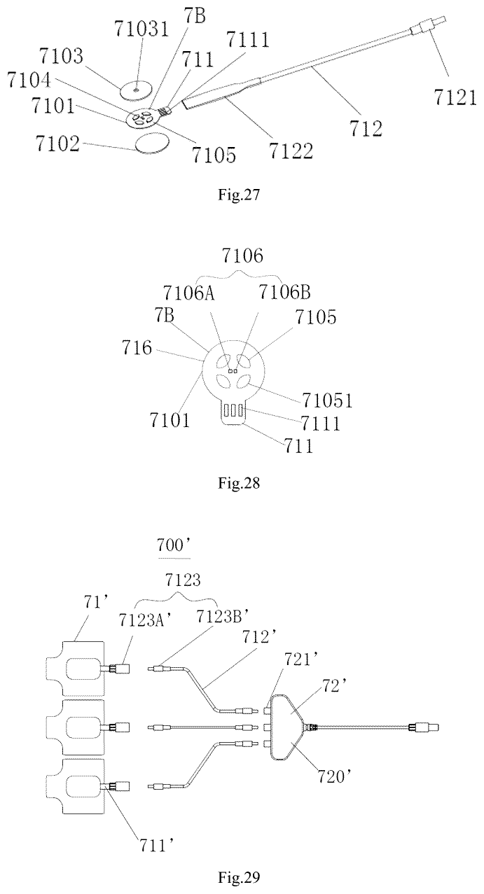

FIG. 27.

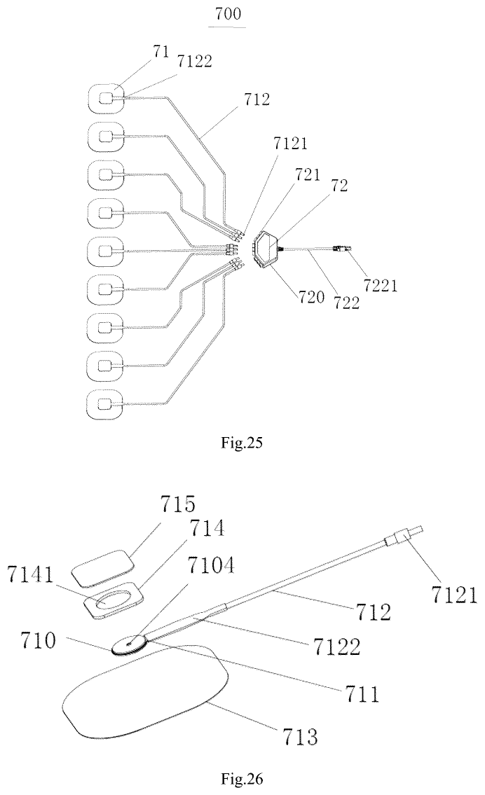

FIG. 29 is an exploded view of an alternate embodiment of the insulated electrode of the fourth embodiment shown in FIG. 25.

FIG. 30 a three-dimensional exploded view of the electrode sheet shown in FIG. 29.

FIG. 31 is a three-dimensional assembled view of a fifth embodiment of an insulated electrode of the tumor treating fields system of the present application.

FIG. 32 is a three-dimensional exploded view of the insulated electrode shown in FIG. 31.

FIG. 33 is a three-dimensional exploded view of a wire and an electrical functional component of the insulated electrode shown in FIG. 32.

FIG. 34 is a planar diagram of a flexible circuit board of the insulated electrode shown in FIG. 33.

FIG. 35 is a planar diagram from another perspective of the flexible circuit board of the insulated electrode shown in FIG. 33.

FIG. 36 is a three-dimensional view of a dielectric element of the insulated electrode shown in FIG. 33.

FIG. 37 is a schematic block diagram of an embodiment of an electric field generator of a tumor treating fields system of the present application.

FIG. 38 is similar to FIG. 37, and is a schematic block diagram of another embodiment of an electric field generator of a tumor treating fields system of the present application.

FIG. 39 is a schematic block diagram of a tumor treating fields system including the electric field generator shown in FIG. 37 or FIG. 38.

FIG. 40 is a flowchart of a method of applying alternating current signals by the tumor treating fields system shown in FIG. 39.

FIG. 41 is a partial flowchart of controlling the electric field generator to apply alternating current signals to the insulated electrode in step 8420 shown in FIG. 40.

FIG. 42 is a further flowchart of controlling the electric field generator to apply alternating current signals to the insulated electrode in step 8420 shown in FIG. 40.

FIG. 43 is a flowchart of an operation of applying alternating current signals by the tumor treating fields system shown in FIG. 39.

FIG. 44 is similar to FIG. 39, and is a structural schematic block diagram of another embodiment of a tumor treating fields system of the present application.

FIG. 45 is planar diagram of the insulated electrode shown in FIG. 44.

FIG. 46 is a three-dimensional exploded view of an electrode sheet of the insulated electrode shown in FIG. 45.

FIG. 47 is a schematic block diagram of a circuit connection of the tumor treating fields system shown in FIG. 44, which only illustrates a circuit connection between one insulated electrode and an adapter.

FIG. 48 is a schematic block diagram of the internal structure of the adapter shown in FIG. 47.

FIG. 49 is a schematic block diagram of a logic circuit of the adapter shown in FIG. 48, in which a serial communication circuit, a signal processor, and a digital-to-analog converter of the adapter are independent modules.

FIG. 50 is a flowchart of a method of the tumor treating fields system shown in FIG. 44 applying alternating current signals.

FIG. 51 is an operation flowchart of controlling to apply alternating current signals in the step 2 shown in FIG. 50.

FIG. 52 is a further operation flowchart of controlling to apply alternating current signals in the step 2 shown in FIG. 50.

### DETAILED DESCRIPTION OF EMBODIMENTS

Exemplary embodiments will be described in detail herein, examples of which are illustrated in the accompanying drawings. When the following description refers to the drawings, unless otherwise indicated, the same numbers in different drawings indicate the same or similar elements. The embodiments described in the following exemplary embodiments do not represent all embodiments consistent with the present disclosure. Rather, they are merely examples of apparatus, systems, devices, and methods consistent with some aspects of the present disclosure.

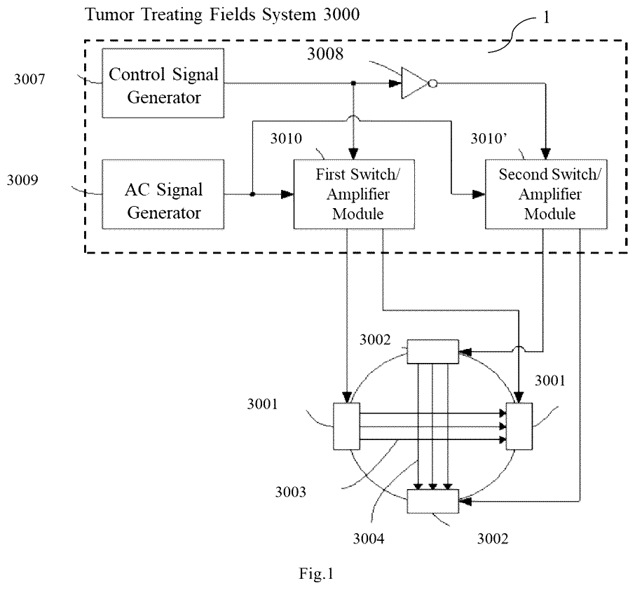

FIG. 1 illustrates a block diagram of an embodiment of a tumor treating fields system 3000 of the present application. The tumor treating fields system 3000 includes a first pair of insulated electrodes 3001, a second pair of insulated electrodes 3002, and a tumor treating fields apparatus (not labeled) electrically connected with the first pair of insulated electrodes 3001 and the second pair of insulated electrodes 3002. The tumor treating fields apparatus (not labeled) applies alternating current signals for tumor treatment to the first pair of insulated electrodes 3001 and the second pair of insulated electrodes 3002. The tumor treating fields apparatus (not labeled) includes an electric field generator (not shown) and an adapter (not shown) electrically connected with the electric field generator (not shown). The first pair of insulated electrodes 3001 and the second pair of insulated electrodes 3002 may be directly electrically connected with the electric field generator (not shown), or may be first electrically connected with the adapter (not shown), and then electrically connected with the electric field generator (not shown) through the adapter (not shown).

The tumor treating fields apparatus (not labeled) includes: a control signal generator 3007; an inverter 3008 electrically connected with the control signal generator 3007; an AC signal generator 3009; a first switch/amplifier module 3010 electrically connected with both the AC signal generator 3009 and the control signal generator 3007; and a second switch/amplifier module 3010′ electrically connected with both the AC signal generator 3009 and the inverter 3008. In one embodiment, the control signal generator 3007, the inverter 3008, the AC signal generator 3009, the first switch/amplifier module 3010 and the second switch/amplifier module 3010′ may all be arranged in an electric field generator (not shown) electrically connected with the first pair of insulated electrodes 3001 and the second pair of insulated electrodes 3002. In another embodiment, the control signal generator 3007, the inverter 3008 and the AC signal generator 3009 are arranged in an electric field generator (not shown), and the first switch/amplifier module 3010 and the second switch/amplifier module 3010′ are both arranged in the adapter (not shown).

In one embodiment, the first switch/amplifier module 3010 may be divided into two elements: a first switch arranged in the adapter (not shown), and an amplifier arranged in the electric field generator (not shown). The second switch/amplifier module 3010′ may also be divided into two elements a second switch and an amplifier, and the second switch is arranged in the adapter and the amplifier is arranged in the electric field generator (not shown). In another embodiment, the AC signal generator 3009, the control signal generator 3007, the inverter 3008 and the amplifiers are all arranged in the electric field generator (not shown), and the first switch and the second switch are both arranged in the adapter (not shown).

The AC signal generator 3009 is used to output sinusoidal signals with adjustable frequency and adjustable amplitude. In the present embodiment, the control signal generator 3007 is a square wave generator generating square wave signals, and the inverter 3008 is used to invert square wave signals from the control signal generator 3007. A control terminal of the first switch/amplifier module 3010 is directly connected with the control signal generator 3007, and a control terminal of the second switch/amplifier module 3010′ is connected with the control signal generator 3007 through the inverter 3008. Input terminals of the first switch/amplifier module 3010 and the second switch/amplifier module 3010′ are both connected with the AC signal generator 3009. An output terminal of the first switch/amplifier module 3010 is connected to the first pair of insulated electrodes 3001, and an output terminal of the second switch/amplifier module 3010′ is connected to the second pair of insulated electrodes 3002. The first switch/amplifier module 3010 and the second switch/amplifier module 3010′ have the function of signal amplification, and also serve as switches. The control signal generator 3007 controls turning on of the first switch/amplifier module 3010 and the second switch/amplifier module 3010′, so that AC signals generated by the AC signal generator 3009 are applied to the first pair of insulated electrodes 3001 and the second pair of insulated electrodes 3002.

When the first pair of insulated electrodes 3001 are turned on, a first electric field 3003 in a first direction is generated between in the first pair of insulated electrodes 3001, and when the second pair of insulated electrodes 3002 are turned on, a second electric field 3004 in a second direction is generated between the second pair of insulated electrodes 3002. The first pair of insulated electrodes 3001 and the second pair of insulated electrodes 3002 are arranged in such a way that the electric field directions of the first electric field 3003 and the second electric field 3004 are perpendicular to each other. Each insulated electrode in the first pair of insulated electrodes 3001 and the second pair of insulated electrodes 3002 includes: an electrical functional component, and a backing that supports the electrical functional component. Preferably, the backing has an adhesive layer. The adhesive layer is attached to a patient's head to place the electrical functional component on the surface of the patient's head. The first pair of insulated electrodes 3001 and the second pair of insulated electrodes 3002 are controlled to be alternately turned on, forming alternating treating electric fields acting on a target area, that is, the first electric field 3003 and the second electric field 3004 that applied alternately.

As one embodiment, the AC signal generator 3009 generates an alternating current signal with intermediate frequency of 200 KHz. The control signal generator 3007 outputs a square wave having a first output state with a high level of 1 and a second output state with a low level of 0. In another embodiment, the AC signal generator 3009 may also generate an alternating current signal with intermediate frequency of 150 KHz.

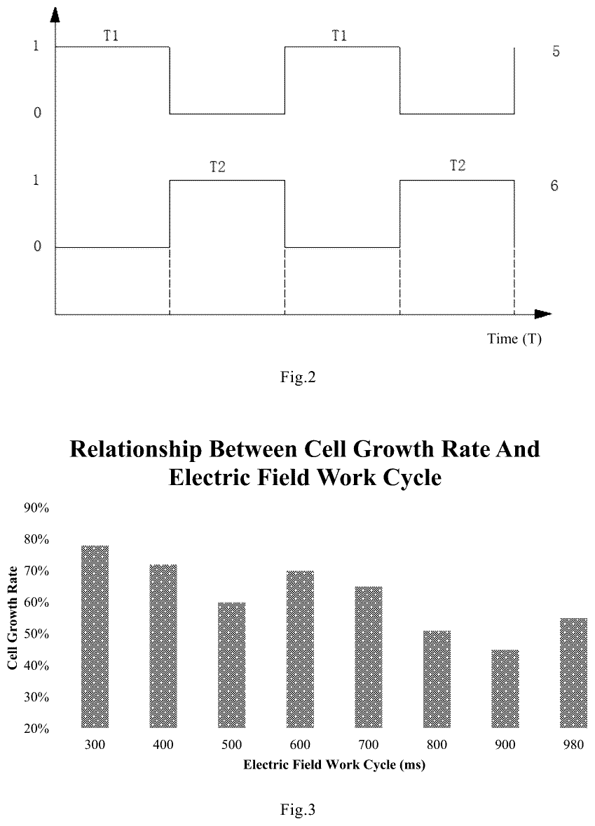

FIG. 2 is a schematic diagram of control signals for turning on or off the first electric field 3003 and the second electric field 3004 in the tumor treating fields system shown in FIG. 1. A control signal input to the first switch/amplifier module 3010 by the control signal generator 3007 is similar to the signal 3005 in FIG. 2 used to turn on and off the first electric field 3003. Due to the arrangement of the inverter 3008, a control signal received by the second switch/amplifier module 3010′ is similar to the signal 3006 in FIG. 2 used to turn on and off the second electric field 3004. The AC signal generator 3009 generating an alternating current signal with intermediate frequency of 200 KHz is taken as an example for illustration.

During the first time period t1, when the control signal generator 3007 outputs a control signal with a first output state, the first switch/amplifier module 3010 is turned on and controls the AC signal on the first pair of insulated electrodes 3001. A first AC signal with a frequency of 200 KHZ is applied to conductors of the first pair of insulated electrodes 3001. A first electric field 3003 with an intensity of at least 1V/cm is generated in a target induction area. Meanwhile, the AC signal on the second pair of insulated electrodes 3002 is turned off, and the second electric field 3004 is turned off. At this point, the signal 3005 is at the high level 1, and the signal 3006 is at the low level 0.

During the second time period t2, when the control signal generator 3007 outputs a control signal with a second output state, the second switch/amplifier module 3010′ is turned on and controls turning on the AC signal on the second pair of insulated electrodes 3002. A second AC signal with a frequency of 200 KHZ is applied to conductors of the second pair of insulated electrodes 3002. A second electric field 3004 with an intensity of at least 1V/cm is generated in the target induction area. Meanwhile, the AC signal on the first pair of insulated electrodes 3001 is turned off, and the first electric field 3003 is turned off. At this point, the signal 3005 is at the low level 0, and the signal 3006 is at the high level 1.

The first time period t1 is a duration time of the control signal of the control signal generator 3007 in the first output state, is a continuous conducting duration of the first electric field 3003 in each work cycle, and is also a turn-off duration of the second electric field 3004. The second time period t2 is a duration time of the control signal of the control signal generator 3007 in the second output state, is a continuous conducting duration of the second electric field 3004 in each work cycle, and is also a turn-off duration of the first electric field 3003. In the present embodiment, the first and second time periods t1, t2 are the same and the first and second time periods t1, t2 each occupies half of the work cycle of the control signals of the control signal generator 3007.

By controlling the first switch/amplifier module 3010 and the second switch/amplifier module 3010′, the control signal generator 3007 may switch the AC signal with intermediate frequency of 200 KHz generated by the AC signal generator 3009 between the first pair of insulated electrodes 3001 and the second pair of insulated electrodes 3002, so that the first electric field 3003 and the second electric field 3004 are alternately applied to the target induction area.

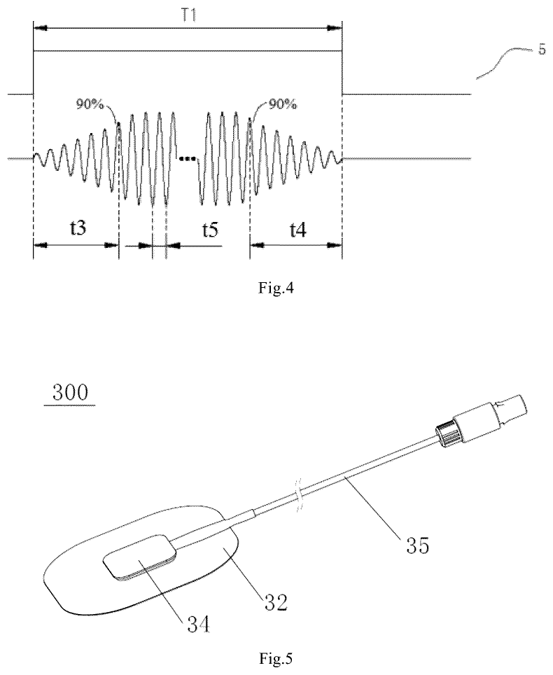

FIG. 3 shows an effect of applying electric fields with different work cycles on cell proliferation during glioma cell culture. The applied electric fields switch at different rates between different directions and thus the inhibitory effects of tumor treating fields therapy on proliferating cells in tissue culture and malignant cells in experimental animals are different.

In the experiment, glioma cells were cultured in a petri dish, and two pairs of mutually perpendicular alternating current signals with frequencies of 200 KHz were applied around the glioma cells. The proliferation of the cells was observed by changing the switching rate of the first electric field 3003 and the second electric field 3004. As shown in FIG. 3, the first electric field 3003 is switched to the second electric field 3004 after working for time length of the first time period t1, and the second electric field 3004 is switched to the first electric field 3003 after working for time length of the second time period t2, and so on, where the first and second time periods t1, t2 are the same and each is half of a cycle of the control signals of the control signal generator 3007. The experimental results show that, when the first and second time periods t1. 12 are within a range from 400 ms to 980 ms, the effects of inhibiting cell proliferation are superior to other rates. Preferably, when the first and second time periods t1, t2 are about 500 ms or between 700 ms and 980 ms, the effects of inhibiting cell proliferation are much better. In the present embodiment. U87MG glioma is used as cell tissue for culture. However, the effect of switching rates on inhibiting cell proliferation is not limited to that cell. Other rapidly proliferating cells may also be suitable.

Since there are non-pure resistive devices in the system, the spike caused by the non-pure resistive devices needs to be suppressed for biological applications. In addition to using insulated electrodes as barriers, preferably, such a phenomenon can be effectively avoided by controlling the climbing rate of the AC signal generated by the AC signal generator 3009 when it is turned on and off. FIG. 4 illustrates an AC signal applied to the first pair of insulated electrodes 3001, in which the climbing rate of the AC signal at the time of turning on and off has been optimized.

During the first time period t1, the AC signal generator 3009 applies the first AC signal on the first pair of insulated electrodes 3001, and generates the first electric field 3003 between the first pair of insulated electrodes 3001. In the initial process of the formation of the first AC signal, the voltage of the first AC signal is increased in a step-up manner. That is, during the switch-to-turn-on period t3, the AC voltage amplitude of the first AC signal applied to the first pair of insulated electrodes 3001 is gradually increased from 0V to a specific value in the step-up manner. The specific value is 90% of a peak value of the target voltage amplitude. The peak value of the target voltage amplitude is a peak value of the output AC voltage amplitude set by the electric field generator (not shown). The first AC signal also has several stable-output-AC-voltage periods 15 between the switch-to-turn-on period t3 and the switch-to-turn-off period t4. During the stable-output-AC-voltage period 15, the AC voltage value of the first AC signal applied to the first pair of insulated electrodes 3001 is between the specific value and the peak value of the output AC voltage amplitude set by the electric field generator (not shown). During the switch-to-turn-off period t4, the AC voltage of the first AC signal applied to the first pair of insulated electrodes 3001 is gradually and slowly reduced from the specific value to 0V in a step-down manner. Similarly, during the second time period t2, the AC signal generator 3009 applies the second AC signal to the second pair of insulated electrodes 3002, and generates the second electric field 3004 between the second pair of insulated electrodes 3002. In the initial process of the formation of the second AC signal, the voltage of the second AC signal is increased in a step-up manner. That is, during the switch-to-turn-on period t3, the AC voltage amplitude of the second AC signal applied to the second pair of insulated electrodes 3002 is gradually increased from 0V to a specific value in the step-up manner. The specific value is 90% of a peak value of the target voltage amplitude. The peak value of the target voltage amplitude is a peak value of the output AC voltage amplitude set by the electric field generator (not shown). The second AC signal also has several stable-output-AC-voltage periods 15 between the switch-to-turn-on period t3 and the switch-to-turn-off period t4. During the stable-output-AC-voltage period t5, the AC voltage value applied to the second pair of insulated electrodes 3002 is between the specific value and the peak value of the output AC voltage amplitude set by the electric field generator (not shown). During the switch-to-turn-off period t4, the AC voltage of the second AC signal applied to the second pair of insulated electrodes 3002 is gradually and slowly reduced from the specific value to 0V. The switching of turning on the first AC signal applied to the first pair of insulated electrodes 3001 when the AC voltage of the second AC signal applied to the second pair of insulated electrodes 3002 is reduced to 0V may effectively avoid the problem that the AC signal generator 3009 applies the AC signals to the first pair of insulated electrodes 3001 and the second pair of insulated electrodes 3002 at the same time which is caused because the AC voltage of the second AC signal applied to the second pair of insulated electrodes 3002 is switched without being reduced to 0V when the second AC signal applied to the second pair of insulated electrodes 3002 is cut off. Therefore, the situation where the first electric field 3003 and the second electric field 3004 coexist and overlap is avoided.

The switch-to-turn-on period t3 and the switch-to-turn-off period t4 are usually within 10% of the time length of the first or second time period t1, t2, so as to avoid the peak signal generated by a sudden change of alternating current signals when switching to turn-on or switching to turn-off impact and damage the control signal generator 3007 or other electronic components. At the same time, the time reaching the electric field intensity for tumor treatment may be ensured as long as possible during each work cycle, so as to ensure the effect of tumor treating fields therapy. In the present embodiment, the sum of the switch-to-turn-on period t3, the switch-to-turn-off period t4, and the several stable-output-AC-voltage periods 15 of the alternating current signal is equal to the working time length of the first or second time period t1, t2 in each cycle of the alternating current signal. During the first time period t1, the first electric field 3003 generated between the first pair of insulated electrodes 3001 is turned on, and the second electric field 3004 generated between the second pair of insulated electrodes 3002 is turned off: while during the second time period t2, the first electric field 3003 generated between the first pair of insulated electrodes 3001 is turned off, and the second electric field 3004 generated between the second pair of insulated electrodes 3002 is turned on, thereby completing a cyclic switching.

Based on the above description, the AC signal generator 3009 of the tumor treating fields system 3000 of the present application generates alternating current signals with a specific intermediate frequency of 200 KHz or 150 KHz which are alternately applied to the two pairs of insulated electrodes 3001 and 3002 to form two perpendicular electric fields with an intensity of 1V/cm applied to the target sensing area, and the control signals generated by the control signal generator 3007 are switched between the first output state and the second output state thereof, so that the system 3000 can realizes the switching between the first AC signal generated between the first pair of insulated electrodes 3001 and the second AC signal generated between the second pair of insulated electrodes 3002, and the durations of the first output state and the second output state are between 400 ms and 980 ms, a better effect of inhibiting tumor cell proliferation may be achieved.

The four insulated electrodes of the first pair of insulated electrodes 3001 and the second pair of insulated electrodes 3002 in the tumor treating fields system 3000 of the present embodiment have the same structure. The insulated electrodes of the present application may have different implementations. The insulated electrodes of the present application are provided with the following various implementations.

A first embodiment of the insulated electrode

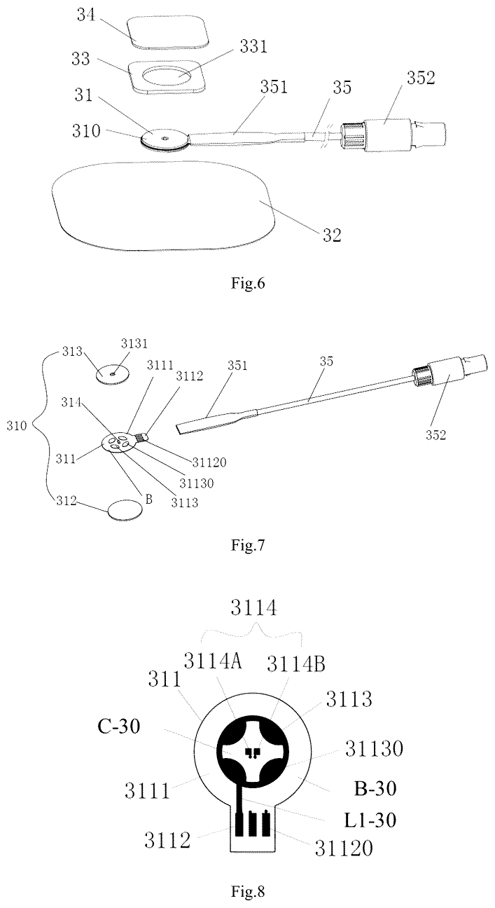

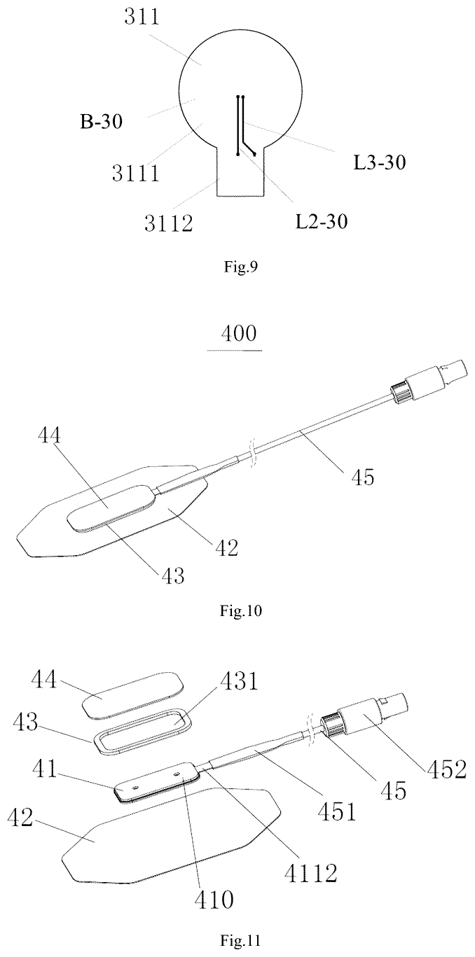

Referring to FIG. 5 to FIG. 9, the insulated electrode 300 of the present embodiment includes: a backing 32: an electrical functional component 31 adhered to the backing 32: a support member 33 adhered to the backing 32: an adhesive 34 that covers the site corresponding to the electrical functional component 31 and the support member 33 and adheres to the body surface skin corresponding to the patient's tumor site; and a wire 35 electrically connected to the electrical functional component 31. The insulated electrode 300 is attached to the body surface corresponding to a patient's tumor site through the backing 32, and an alternating electric signal is applied to the patient's tumor site through the electrical functional component 31 to interfere or prevent the mitosis of the patient's tumor cells, so as to achieve the purpose of treating the tumor.

The electrical functional component 31 includes a single electrode unit 310 in a circular sheet shape and a wiring part 3112 connected with the electrode unit 310. The wiring part 3112 is welded with the wire 35 to realize the electrical connection between the electrical functional component 31 and the wire 35. A plurality of golden fingers 31120 are arranged on the surface of the wiring part 3112. In the present embodiment, the plurality of golden fingers 31120 are arranged on the surface of the wiring part 3112 facing the skin. The wire 35 is welded with the golden fingers 31120 of the wiring part 3112, and the periphery of the welding point is covered with a heat shrinkable sleeve 351. The heat shrinkable sleeve 351 provides support and insulation protection for the connecting point between the wire 35 and the wiring part 3112 of the electrical functional component 31, so as to prevent the connecting point between the wire 35 and the wiring part 3112 of the electrical functional component 31 from being broken. Meanwhile, it may also be dustproof and waterproof. The end of the wire 35 away from the wiring part 3112 is configured with a plug 352 electrically connected to an electric field generator (not shown). One end of the wire 35 is electrically connected to the golden fingers 31120 of the wiring part 3112, and the other end of the wire 35 is electrically connected with the electric field generator (not shown) through the plug 352, so as to provide the insulated electrode 300 with alternating current signals for tumor treatment during the tumor treating fields therapy.

The electrode unit 310 includes: a main body part 3111: an insulating plate 312 arranged at one side of the main body part 3111 away from the human skin: a dielectric element 313 arranged at one side of the main body part 3111 facing the human skin; and a temperature sensor 314 arranged on the main body part 3111 and located at the same side as the dielectric element 313. The main body part 3111, the insulating plate 312 and the dielectric element 313 are all in a circular sheet structure, and have centers located on the same straight line. The wiring part 3112 is laterally extended from the main body part 3111 of the electrode unit 310.

The main body part 3111 includes an insulating substrate B-30 and three paths of conductive traces L-30 embedded in the insulating substrate B-30. The three paths of conductive traces are: a first conductive trace L1-30 arranged on a side of the insulating substrate B-30 close to the dielectric element 313; and a second conductive trace L2-30 and a third conductive trace L3-30 located on a side of the insulating substrate B-30 close to the insulating plate 312. The diameter of the main body part 3111 is greater than 20 mm, preferably 21 mm. The main body part 3111 is provided with a conductive pad 3113 that is exposed from the insulating substrate B-30 and is electrically connected with the first conductive trace L1-30. The conductive pad 3113 may be welded with the dielectric element 313 to assemble the dielectric element 3113 on the main body part 3111. The conductive pad 3113 may be completely covered by the dielectric element 313, so that the conductive pad 3113 and the dielectric element 313 can be welded by solder (not shown). The center of the conductive pad 3113 is located on the center line of the main body part 3111. The conductive pad 3113 includes a plurality of conductive cores 31130 center-symmetrically arranged, which may effectively prevent the offset of the position of the dielectric element 313 caused by the stacking of the solder (not shown) during the welding process. The top surfaces of the plurality of conductive cores 31130 are located on the same plane, which may avoid pseudo soldering when welding with the dielectric element 313. The plurality of conductive cores 31130 are all connected with the first conductive trace L1-30. The plurality of conductive cores 31130 are connected in series by the first conductive trace L1-30.

In the present embodiment, the conductive pad 3113 of the main body part 3111 includes four petal-shaped conductive cores 31130 center-symmetrically arranged and arranged at intervals. The conductive cores 31130 are arranged in a multi-point interval mode, so that the consumption of cop foil for manufacturing the conductive core 31130 can be reduced. At the same time, the amount of solder (not shown) used for welding the conductive cores 31130 and the dielectric element 313 can be saved, reducing the manufacturing cost. The four conductive cores 31130 of the conductive pad 3113 are all in petal-shaped structures. Each conductive core 31130 includes an inner arc (not labeled) and an outer arc (not labeled) connected end to end. The inner arcs (not labeled) and the outer arcs (not labeled) of the conductive cores 31130 are axial-symmetrically arranged. Inner arcs (not labeled) of the four conductive cores 31130 of the same conductive pad 3113 are all recessed in a direction toward the center of the conductive pad 3113. Outer arcs (not labeled) of the four conductive cores 31130 of the same conductive pad 3113 all protrude in a direction away from the center of the conductive pad 3113.

The four conductive cores 31130 constituting the conductive pad 3113 are both center-symmetrically arranged and axial-symmetrically arranged, and each conductive core 31130 is also axial-symmetrically arranged, so that when the four conductive cores 31130 of the conductive pad 3113 of the main body part 3111 are welded with the dielectric element 313, the stress balance of each welding point is guaranteed, ensuring an overall welding balance of the dielectric element 313 and improving the welding quality. Therefore, it may avoid an inclination of the dielectric element 313 caused by the unbalanced welding stress, which may result in a weak strength and easy fracture of the welding point on a side with larger spacing between the dielectric element 313 and the main body part 3111. At the same time, it may also avoid affecting the fit of the insulated electrode 300. Outer arcs (not labeled) of the four conductive cores 31130 of the conductive pad 3113 are substantially located on a same circumference, and the four conductive cores 31130 are connected in series by the first conductive trace L1-30. Every two of the four conductive cores 31130 of the conductive pad 3113 are arranged at intervals, and a spacing C-30 is formed between two adjacent conductive cores 31130. The four spacings C-30 are substantially in a cross shape. Adjacent spacings C-30 are communicated. The extension direction of the two opposite spacings C-30 is consistent with the extension direction of the wiring part 3112.

The main body part 3111 further includes a pair of pads 3114 that is exposed from the insulating substrate B-30 thereof. The pair of pads 3114 can be welded with the corresponding part of the temperature sensor 314 to realize the electrical connection between the temperature sensor 314 and the main body part 3111. The two pads 3114 are surrounded by four conductive cores 31130 of the conductive pads 3113. The two pads 3114 are substantially located on the symmetrical center of the plurality of conductive cores 31130. One pad 3114 is connected with the second conductive trace L2-30, and the other pad is connected with the third conductive trace L3-30. The first pad 3114A is the one of the two pads that is connected with the second conductive trace L2-30, and the second pad 3114B is the other one of the two pads that is connected with the third conductive trace L3-30. The temperature sensor 314 has a signal terminal (not shown) and a ground terminal (not shown). The first pad 3114A is welded with the ground terminal (not shown) of the temperature sensor 314, and the second pad 3114B is welded with the signal terminal (not shown) of the temperature sensor 314.

The insulating plate 312 is made of an insulating material. Preferably, the insulating plate 312 is made of an epoxy glass cloth laminate plate. The insulating plate 312 is adhered to a surface of the main body part 3111 away from human skin by sealant (not shown), which can enhance the strength of the main body part 3111, provide a flat welding plane for the welding operation between the main body part 3111 and the dielectric element 313, and improve the product yield rate. At the same time, the insulating plate 312 may also isolate the water vapor in the air on the side of the insulated electrode 300 away from the skin from the solder (not shown) located between the main body part 3111 and the dielectric element 313, so as to prevent the water vapor from eroding the solder (not shown) between the main body part 3111 and the dielectric element 313, which may affect the electrical connection between the main body part 3111 and the dielectric element 313.

The dimension of the insulating plate 312 is substantially the same as the dimension of the main body part 3111, so as to prevent the sealant (not shown) from climbing to the side of the main body part 3111 facing the human skin due to capillary effect when the insulating plate 312 is adhered to the side of the main body part 3111 away from the human skin by sealant (not shown), which may affect the filling of the sealant (not shown) in a gap (not shown) formed by welding the dielectric element 313 and the main body part 3111, resulting the existence of cavities in the sealant (not shown). This may further prevent the sealant (not shown) from bursting due to the rapid expansion of water vapor caused by a large difference in thermal expansion coefficients between the sealant (not shown) and the water vapor in the cavities during high-temperature curing, which may lead to popcorn phenomenon and damage the products.

The dielectric element 313 is made of a material with a high dielectric constant, and has conductive characteristics of blocking the conduction of direct current and allowing alternating current to pass through, which may ensure human safety. Preferably, the dielectric element 313 is made of a dielectric ceramic sheet. The dielectric element 313 has an annular structure, with a perforations 3131 extended therethrough in the middle corresponding to the pair of pads 3114 of the main body part 3111 for accommodating the temperature sensor 314. An annular metal layer (not shown) is attached to the surface of the dielectric element 313 facing the main body part 3111. A point-to-side welding is formed between the metal layer (not shown) of the dielectric element 313 and the conductive cores 31130 of the conductive pad 3113 of the main body part 3111, so that it is more convenient to weld without requiring higher welding alignment accuracy: The inner ring of the metal layer (not shown) of the dielectric element 313 and the edge of the perforation 3131 of the dielectric element 313 are arranged at intervals, so as to prevent the solder (not shown) between the metal layer (not shown) of the dielectric element 313 and the main body part 3111 from spreading in a direction toward the perforation 3131 of the dielectric element 313 when heated and melted, which may result in a short circuit of the temperature sensor 314. The outer ring of the metal layer (not shown) of the dielectric element 313 and the outer edge of the dielectric element 313 are also arranged at intervals, so as to prevent the solder (not shown) between the metal layer (not shown) of the dielectric element 313 and the main body part 3111 from overflowing to the outside of the main body part 3111 when heated and melted, which may result in a direct current that is not hindered by the dielectric element 313 passes through and acts on the patient's body surface when the insulated electrode 300 is attached to the body surface of the patient's tumor site.

The gap (not shown) formed by welding the dielectric element 313 and the main body part 3111 is filled with sealant (not shown) to protect the solder (not shown) between the dielectric element 313 and the main body part 3111, so as to prevent the dielectric element 313 from being affected by external forces, which leads to the fracture of the welding part, and further leads to the failure of the alternating electric fields to be applied to the patient's tumor site through the dielectric element 313. At the same time, it can also prevent the water vapor in the air from entering the gap (not shown) and eroding the solder (not shown) between the dielectric element 313 and the main body part 3111, which may affect the electrical connection between the dielectric element 313 and the main body part 3111. The outer diameter of the dielectric element 313 is slightly smaller than the diameter of the main body part 3111. When filling the sealant (not shown), the sealant (not shown) can be filled into the gap (not shown) along the edge of the main body part 3111 located outside the dielectric element 313 by capillary phenomenon, which is beneficial to the filling of the sealant (not shown) in the gap (not shown) formed by welding the dielectric element 313 and the main body part 3111. When filling the sealant (not shown) in the gap (not shown) formed by welding the dielectric element 313 and the main body part 3111, the air in the gap (not shown) may be exhausted from the perforation 3131 of the dielectric element 313, so as to prevent the sealant (not shown) filled in the gap (not shown) from generating cavities, and thus improve the product quality.

The temperature sensor 314 is arranged on the main body part 3111 by welding the ground terminal (not shown) thereof with the first pad 3114A arranged on the main body part 3111, and welding the signal terminal (not shown) thereof with the second pad 3114B arranged on the main body part 3111. The first pad 3114A of the main body part 3111 is connected with the second conductive trace L2-30, the second pad 3114B is connected with the third conductive trace L3-30, and the first pad 3114A is welded with the ground terminal (not shown) of the temperature sensor 314, the second pad 3114B is welded with the signal terminal (not shown) of the temperature sensor 314, thereby the ground terminal (not shown) of the temperature sensor 314 is electrically connected with the second conductive trace L2-30 of the main body part 3111, and the signal terminal (not shown) of the temperature sensor 314 is electrically connected with the third conductive trace L3-30 of the main body part 3111. That is, the temperature sensor 314 transmits signals through the second conductive trace L2-30 and the third conductive trace L3-30. The temperature sensor 314 is welded on the main body part 3111 and is accommodated in the perforation 3131 of the dielectric element 313. Preferably, the temperature sensor 314 is a thermistor. The temperature sensor 314 is used to monitor the temperature of the adhesive 34 covering the surface of the dielectric element 313 of the electrical functional component 31 facing the human skin, and further detect the temperature of the human skin to which the adhesive 34 is attached. When the temperature monitored by the temperature sensor 314 exceeds the upper limit of the safe temperature of human body, the tumor treating fields system 3000 may reduce the alternating voltage of the alternating electrical signals or turn off the alternating electrical signals applied to the insulated electrode 300 in time, so as to avoid cryogenic burns of human body. The temperature sensor 314 is welded to the main body part 3111 through the pair of pads 3114 of the main body part 3111, and is then sealed by sealant (not shown), so as to prevent the water vapor from eroding the temperature sensor 314 and causing a failure of the temperature sensor 314.

The wiring part 3112 has the same structure as the main body part 3111, and also has a corresponding insulating substrate B-30 and three paths of the conductive traces L-30 embedded in the insulating substrate B-30. The three paths of the conductive traces L-30 of the wiring part 3112 are also electrically connected to the corresponding conductive traces L-30 of the main body part 3111, respectively. The wiring part 3112 has three golden fingers 31120, which are exposed from the side of the insulating substrate B-30 close to the dielectric element 313. The three paths of the conductive traces L-30 of the wiring part 3112 are electrically connected with the golden fingers 31120, respectively. The three paths of the conductive traces L-30 of the wiring part 3112 are also the first conductive trace L1-30, the second conductive trace L2-30, and the third conductive traces L3-30, respectively. The first conductive trace L1-30 of the wiring part 3112 is extended from the first conductive trace L1-30 of the main body part 3111. The second conductive trace L2-30 of the wiring part 3112 is extended from the second conductive trace L2-30 of the main body part 3111. The conductive traces L3-30 of the 25 wiring part 3112 is extended from the third conductive trace L3-30 of the main body part 3111.

The wiring part 3112 realizes the electrical connection with the conductive pad 3113 of the main body part 3111 by connecting the first conductive trace L1-30 thereof with the first conductive trace L1-30 of the main body part 3111, and connecting the first conductive trace L1-30 of the main body part 3111 with the conductive pad 3113 on the main body part 3111, and further realizes the electrical connection with the dielectric element 313 by welding the conductive pad 3113 of the main body part 3111 with the dielectric element 313. The wiring part 3112 realizes the electrical connection with the first pad 3114A of the main body part 3111 by connecting the second conductive trace L2-30 thereof with the second conductive trace L2-30 of the main body part 3111, and connecting the second conductive trace L2-30 of the main body part 3111 with the first pad 3114A on the main body part 3111, and further realizes the electrical connection with the ground terminal (not shown) of the temperature sensor 314 by welding the first pad 3114A with the ground terminal (not shown) of the temperature sensor 314. The wiring part 3112 realizes the electrical connection with the second pad 3114B of the main body part 3111 by connecting the third conductive trace L3-30 thereof with the third conductive trace L3-30 of the main body part 3111, and connecting the third conductive trace L3-30 of the main body part 3111 with the second pad 3114B, and further realizes the electrical connection with the signal terminal (not shown) of the temperature sensor 314 by welding the second pad 3114B with the signal terminal (not shown) of the temperature sensor 314.

The main body part 3111 and the wiring part 3112 together constitute the flexible circuit board 311 of the electrical functional component 31. The respective insulating substrates B-30 of the main body part 3111 and the wiring part 3112 together constitute the insulating substrate B-30 of the flexible circuit board 311. The conductive traces L-30 of the main body part 3111 and the conductive traces L-30 of the wiring part 3112 are in one-to-one correspondence, and form the conductive trace L-30 of the flexible circuit board 311. The insulating substrate B-30 of the flexible circuit board 311 may isolate the water vapor in the air around the insulated electrode 300 from the solder (not shown) located between the conductive pad 3113 and the dielectric element 313, and prevent the water vapor in the air away from the skin from eroding the solder (not shown) located between the dielectric element 313 and the conductive pad 3113 on the main body part 3111 of the flexible circuit board 311. The insulating substrate B-30 of the flexible circuit board 311 and the insulating plate 312 function as a double isolation, which may prolong the service life of the insulated electrode 300.

In view of the formation of the electrode unit 310, the insulating plate 312 is arranged on the side of the main body part 3111 of the flexible circuit board 311 away from the human skin, the dielectric element 313 is arranged on the side of the main body part 3111 of the flexible circuit board 311 facing the human skin, and the temperature sensor 314 is arranged on the side of the main body part 3111 of the flexible circuit board 311 facing the human skin. The insulating plate 312 and the dielectric element 313 are respectively arranged on opposite sides of the main body part 3111 of the flexible circuit board 311. The first conductive trace L1-30 of the flexible circuit board 311 connects the four spaced conductive cores 31130 of the conductive pad 3113 in series. The second conductive trace L2-30 is electrically connected to the ground terminal (not shown) of the temperature sensor 314 through the first pad 3114A. The third conductive trace L3-30 is electrically connected to the signal terminal (not shown) of the temperature sensor 314 through the second pad 3114B. The first conductive trace L1-30 is located in the insulating substrate B-30 at a layer close to the human skin. The second conductive trace L2-30 and the third conductive trace L3-30 are located in the insulating substrate B-30 at a layer close to the insulating plate 312. In order to facilitate the laying of the conductive traces L-30, the width of the wiring part 3112 is 7 mm to 9 mm. Preferably, the width of the wiring part 3112 is 8 mm.

The golden fingers 31120 of the wiring part 3112, the plurality of conductive cores 31130 of the conductive pad 3113, and the pads 3114 are all exposed from the side of the insulating substrate B-30 of the flexible circuit board 311 close to the dielectric element 313. The golden fingers 31120, the plurality of conductive cores 31130 of the conductive pad 3113, and the pads 3114 are all located on the side of the flexible circuit board 311 close to the patient's body surface. One golden finger 31120 of the wiring part 3112 has one end electrically connected with the dielectric element 313 through the first conductive trace L1-30 connected thereto and the other end welded with the corresponding part of the wire 35, so as to transmit the alternating electrical signal generated by the electric field generator (not shown) to the dielectric element 313. One of the other two golden fingers 31120 of the wiring part 3112 has an end electrically connected to the ground terminal (not shown) of the temperature sensor 314 through the second conductive trace L2-30 connected thereto, and the other one of the other two golden fingers 31120 has one end electrically connected to the signal terminal (not shown) of the temperature sensor 314 through the third conductive trace L3-30 connected thereto. The other ends of that two golden fingers 31120 of the wiring part 3112 are respectively welded 25 with the corresponding parts of the wire 35. Thus, it realizes that the related signals detected by the temperature sensor 314 are transmitted to the electric field generator (not shown) through the second conductive trace L2-30, the third conductive trace L3-30 and the wire 35.

The backing 32 is in a sheet shape, which is mainly made of a flexible and permeable insulating material. The backing 32 is a mesh fabric. Specifically, the backing 32 is a mesh non-woven fabric, which has the characteristics of softness, lightness, moisture-proof and permeability, and can keep the patient's skin surface dry after being attached to the patient's body surface for a long time. The side of the backing 32 facing the patient's body surface is also coated with a biocompatible adhesive (not shown) for tightly adhering the backing 32 to the body surface corresponding to the patient's tumor site. In the present embodiment, the backing 32 is substantially in a cubic sheet structure, and the four corners of the backing 32 are set in a rounded shape. In other implementations, the backing 32 is substantially in a cross-shaped configuration, and the four corners of the backing 32 of are all provided with concave corners (not shown). The concave corner (not shown) located at the corner of the backing 32 communicates with the outside, and is in an “L” shape. An angle between two sides of the backing 32 that forms the concave corner (not shown) is greater than or equal to 90 degrees. When the insulated electrode 300 is attached to the body surface corresponding to the patient's tumor site, the concave corner (not shown) may prevent the corners of the backing 32 from arching and forming wrinkles, and further avoid air entering between the electrode unit 310 and the skin from the wrinkles, which results in cryogenic burns due to the increase of heat generation of the electrical functional component 31 caused by the increase of the impedance between the electrical functional component 31 and the skin.

The support member 33 is adhered to the backing 32 and surrounds the outside of the electrode unit 310. In the middle of the support member 33, a through hole 331 is disposed therethrough for accommodating the electrode unit 310. The support member 33 may be made of foam material. The support member 33 is flush with the surface of a side of the electrode unit 310 away from the backing 32. The support member 33 is flush with the surface of a side of the electrode unit 310 facing the adhesive 34.

The adhesive 34 has double-sided adhesiveness. One surface of the adhesive 34 is adhered to the surface of the support member 33 and the electrode unit 310 away from the backing 32. The other surface of the adhesive 34 is used as an adhesive lay, which is attached to the skin of human body surface to keep the skin surface moist and relieve local pressure. The adhesive 34 may preferably be a conductive adhesive to serve as a conductive medium. With the support of the support member 33, the adhesive 34 has better adhesion to human skin.

The flexible circuit board 311 of the insulated electrode 300 of the present embodiment is only provided with: a path of the first conductive trace L1-30 electrically connected with the dielectric element 313: a path of the second conductive trace L2-30 electrically connected with the ground terminal (not shown) of the temperature sensor 314; and a path of the third conductive trace L3-30 electrically connected with the signal terminal (not shown) of the temperature sensor 314. It realizes to transmit the alternating electrical signals of the electric field generator (not shown) to the dielectric element 313 through the first conductive trace L1-30 to realize the purpose of applying the alternating electrical signal to the patient's tumor site for tumor treatment. At the same time, it realizes the signal transmission between the electric field generator (not shown) and the temperature sensor 314 by electrically connecting the second conductive trace L2-30 and the third conductive trace L3-30 with the temperature sensors 314, respectively. Thus, the difficulty of layering design is low: the structure is simple, the manufacturing process is simple, the manufacturing is easy, and the product manufacturing yield is high, and it may greatly reduce the manufacturing cost. In addition, since the insulated electrode 300 uses a separate electrode unit 310 to apply alternating electrical signals to the patient's tumor site, when it doesn't work properly, it only needs to replace the insulated electrode 300 with the separate electrode unit 310 without needing to scrap the entire insulated electrode containing a plurality of electrode units 310, which can reduce the cost of tumor treatment for a patient. Furthermore, the insulated electrodes 300 of the present embodiment may be freely combined in quantity according to the patient's tumor site and the size of the patient's tumor site, ensuring the coverage area of the insulated electrode 300 for tumor treating fields therapy and ensuring the required electric field strength of the tumor treating fields therapy. Moreover, the relative positions of the plurality of insulated electrodes 300 may also be freely adjusted according to the patient's own physical differences, tumor site, and tumor size, in order to obtain the optimal electric field strength and coverage area for tumor treatment. At the same time, it may allow the skin on the patient's body surface to which the insulated electrodes 300 are attached to breathe freely, avoiding the accumulation of heat on the patient's body surface caused by a long-term tumor treating fields therapy, and being not able to dissipate in time, which causes sweating and clogging pores and results in skin inflammation.

A second embodiment of the insulated electrode

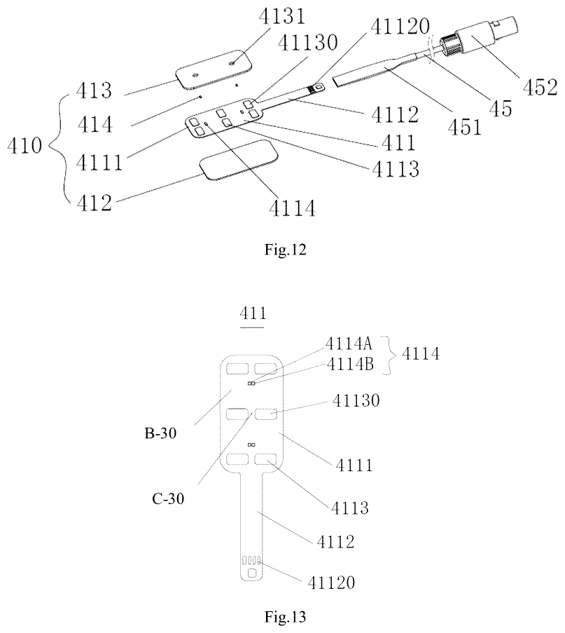

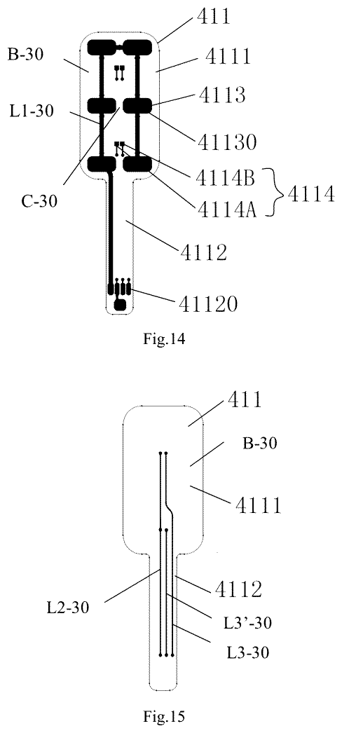

Referring to FIG. 10 to FIG. 15, in the present embodiment, the insulated electrode 400 includes: a backing 42: an electrical functional component 41 adhered to the backing 42: a support member 43 adhered to the backing 42: an adhesive 44 that covers the site corresponding to the electrical functional component 41 and the support member 43 and adheres to the body surface skin corresponding to the patient's tumor site; and a wire 45 electrically connected to the electrical functional component 41. The backing 42 and the support member 43, except for slightly differences in appearance, have the same functions and materials as the backing 32 and the support member 33 of the embodiment of the insulated electrode 300, respectively, and will not be repeated here. Reference may be made to the first embodiment for related contents.

The electrical function component 41 includes a single electrode unit 410 in a rectangular sheet shape and a wiring part 4112 connected with the electrode unit 410. The support member 43 has a through hole 431 disposed therethrough for accommodating the electrode unit 410. The wiring part 4112 is welded with the wire 45 to realize the electrical connection between the electrical functional component 41 and the wire 45. Four golden fingers 41120 are arranged on the surface of the wiring part 4112 facing the skin. The periphery of the welding point of the wire 45 and the golden fingers 41120 of the wiring part 4112 is covered with a heat shrinkable sleeve 451. The end of the wire 45 away from the wiring part 4112 is configured with a first plug 452 electrically connected to an electric field generator (not shown) or an adapter (not shown).

The electrode unit 410 includes: a main body part 4111 arranged at the end of the wiring part 4112: an insulating plate 412 arranged at one side of the main body part 4111 away from the human skin: a dielectric element 413 arranged at one side of the main body part 4111 facing the human skin; and two temperature sensors 414 arranged on the main body part 4111 and located at the same side as the dielectric element 413. The main body part 4111 and the wire 45 are respectively arranged at opposite ends of the wiring part 4112. The dielectric element 413 is provided with two perforations 4131 extended therethrough for accommodating the corresponding temperature sensors 414 respectively, in which the number of the perforations 4131 is the same as the number of the temperature sensors 414. The main body part 4111, the insulating plate 412 and the dielectric element 413 have almost the same shapes, and are all in the structure of a rectangular sheet. The main body part 4111, the insulating plate 412, and the dielectric element 413 are correspondingly arranged along the thickness direction of the main body part 4111, and have centers located on the same straight line. In the present embodiment, the main body part 4111, the insulating plate 412 and the dielectric element 413 are all in the structure of a rectangular sheet with rounded corners. Preferably, the main body part 4111 is in the structure of a rectangular sheet with a dimension of about 43.5 mm×23.5 mm. The wiring part 4112 of the electrical function component 41 is laterally extended from the main body part 4111 of the electrode unit 410. In other implementations, the main body part 4111 may also be in a strip-shaped or band-shaped structure extended from the end of the wiring part 4112.

The main body part 4111 includes an insulating substrate B-30 and four paths of conductive traces L-30 embedded in the insulating substrate B-30. The four paths of conductive traces include: one path of a first conductive trace L1-30 arranged on a side of the insulating substrate B-30 close to the dielectric element 413, one path of a second conductive trace L2-30 arranged on a side of the insulating substrate B-30 close to the insulating plate 412, and two paths of third conductive traces L3-30 and L3′-30 arranged on the same side as the second conductive trace L2-30. The main body part 4111 is centrally provided with a conductive pad 4113 that is exposed from the insulating substrate B-30 and is electorally connected with the first conductive trace L1-30. A metal layer (not shown) is attached to the surface of the dielectric element 413 facing the main body part 4111. The conductive pad 4113 is welded with the metal layer (not shown) of the dielectric element 413 to assemble the dielectric element 413 on the main body part 4111. The conductive pad 4113 may be completely covered by the dielectric element 413, so that the conductive pad 4113 and the dielectric element 413 can be welded by solder (not shown). The center of the conductive pad 4113 is located on the center line of the main body part 4111. The conductive pad 4113 includes a plurality of conductive cores 41130 center-symmetrically arranged, which may effectively prevent the offset of the position of the dielectric element 413 caused by the stacking of the solder (not shown) during the welding process. The top surfaces of the plurality of conductive cores 41130 are located on the same plane, which may avoid pseudo soldering when welding with the dielectric element 413. The plurality of conductive cores 41130 are all connected with a first conductive trace L1-30. The plurality of conductive cores 41130 are connected in series by the first conductive trace L1-30.

In the present embodiment, the conductive pad 4113 of the main body part 4111 is substantially in a rectangle structure, and has symmetry axes respectively coincident with the corresponding symmetry axes of the main body part 4111. The conductive pad 4113 includes six spaced conductive cores 41130 located at four corners thereof and in the middle of two long sides thereof. The conductive cores 41130 are arranged in a multi-point interval mode, so that the consumption of cop foil for manufacturing the conductive cores 41130 can be reduced. At the same time, the amount of solder (not shown) used for welding the conductive cores 41130 and the dielectric element 413 can be saved, reducing the manufacturing cost. Each conductive core 41130 is in a rectangular structure with a dimension of approximately 8 mm×4 mm. Preferably, each conductive core 41130 is constructed in a rectangular shape with rounded corners. The longitudinal axis of each conductive core 41130 is parallel to the extension direction of the wiring part 4112. In other embodiments, each conductive core 41130 of the conductive pad 4113 may also be circular, square, etc.

In the present embodiment, the six conductive cores 41130 constituting the conductive pad 4113 are arranged in a matrix at intervals, and the six conductive cores 41130 are arranged in three rows and two columns along the longitudinal direction of the main body part 4111. There are two conductive cores 41130 in the first row, two conductive cores 41130 in the middle row; and two conductive cores 41130 in the last row: The spacing formed between two columns of conductive cores 41130 is about 2.4 mm, and the spacings between conductive cores 41130 in adjacent rows are all about 12.8 mm. The six conductive cores 41130 constituting the conductive pad 4113 are both center-symmetrically arranged and axial-symmetrically arranged, and each conductive core 41130 is also axial-symmetrically arranged, so that when the six conductive cores 41130 of the main body part 4111 are welded with the dielectric element 413, the stress at each welding point is balanced, ensuring an overall welding balance of the dielectric element 413 and improving the welding quality. Therefore, it may avoid an inclination of the dielectric element 413 caused by the unbalanced welding stress, which may result in a weak strength and easy fracture of the welding point on a side with a larger spacing between the dielectric element 413 and the main body part 4111. At the same time, it may also avoid affecting the fit of the insulated electrode 400.

The six conductive cores 41130 of the conductive pad 4113 are arranged at intervals, and a spacing C-30 is formed between two adjacent conductive cores 41130. Every two of the four conductive cores 41130 located in adjacent rows are arranged at intervals, and the four spacings C-30 located between the four conductive cores 41130 are communicated and substantially arranged in a cross shape. The dimension of the spacing C-30 between two adjacent conductive cores 41130 in the same column is larger than the dimension of the spacing C-30 between two conductive cores 41130 in the same row: Seven spacings C-30 are formed between the six conductive cores 41130, and the seven spacings C-30 are communicated and substantially arranged in a “#” shape. Adjacent spacings C-30 are also arranged in a communicated manner. Among the seven spacings C-30, three of the spacings C-30 located between two adjacent conductive cores 41130 in the same row are at a straight line in a direction consistent with the extension direction of the wiring part 4112.

The main body part 4111 is further provided with two pairs of pads 4114 that are exposed from the insulating substrate B-30, and the two pairs of pads 4114 are respectively welded with corresponding parts of the two corresponding temperature sensors 414, so as to realize the electrical connection between the temperature sensors 414 and the main body part 4111. Each pair of pads 4114 is located at the corresponding communication area of four spacings C-30 formed by four conductive cores 41130 located in adjacent rows. The line connecting respective symmetrical centers of the two pairs of pads 4114 is at a straight line in a direction consistent with the extension direction of the wiring part 4112. The line connecting the two symmetrical centers of the two pairs of pads 4114 is at a straight line coincident with the longitudinal axis of the main body part 4111. The line connecting the two symmetrical centers of the two pairs of pads 4114 is at a straight line coincident with the longitudinal axis of the conductive pad 4113. The four conductive cores 41130 in the first row and the middle row are center-symmetrically arranged, and the four conductive cores 41130 in the middle row and the last row are also center-symmetrically arranged. The two pairs of pads 4114 are arranged offset from the symmetrical center of the four conductive cores 41130 located in two adjacent rows.

Specifically, one pair of pads 4114 is arranged on a side of the symmetrical center of a rectangle away from the wiring part 4112, and the rectangle is formed by the four conductive cores 41130 in the first row and the middle row: The other pair of pads 4114 is arranged on a side of the symmetrical center of a rectangle close to the wiring part 4112, and the rectangle is formed by the four conductive cores 41130 in the middle row and the last row. Each pair of pads 4114 includes a first pad 4114A and a second pad 4114B. The first pad 4114A of each pair of pads 4114 is electrically connected to the second conductive trace L2-30. One of the two second pads 4114B is electrically connected to the third conductive trace L3-30, and the other one of the two second pads 4114B is electrically connected to the third conductive trace L3′-30. The temperature sensor 414 has a signal terminal (not shown) and a ground terminal (not shown). The first pad 4114A is welded with the ground terminal (not shown) of the temperature sensor 414, and the second pad 4114B is welded with the signal terminal (not shown) of the temperature sensor 414.

One of the two temperature sensors 414 is located at the communication area of four spacings C-30 between the four conductive cores 41130 in the first row and the middle row; and the other one of the two temperature sensors 414 is located at the communication area of four spacings C-30 between the four conductive cores 41130 in the middle row and the last row. One temperature sensor 414 located in the surrounding area of the four conductive cores 41130 in the first row and the middle row is located at a side of the symmetrical center of the surrounding area of the four conductive cores 41130 in the first row and the middle row away from the wiring part 4112. The other temperature sensor 414 located in the surrounding area of the four conductive cores 41130 in the middle row and the last row is located at a side of the symmetrical center of the surrounding area of the four conductive cores 41130 in the middle row and the last row close to the wiring part 4112. Both of the two temperature sensors 414 are located in an area surrounded by the conductive pad 4113. Each temperature sensor 414 is welded with the first pad 4114A arranged on the main body part 4111 through the ground terminal (not shown) thereof and is welded with the corresponding second pad 4114B arranged on the main body part 4111 through the signal terminal (not shown) thereof, so as to realize the electrical connection between the temperature sensors 414 and the main body part 4111.

Since the two first pads 4114A of the main body part 4111 are both connected with the second conductive trace L2-30, one of the two second pads 4114B is connected with the third conductive trace L3-30, the other one of the two second pads 4114B is connected with the third conductive trace L3′-30, meanwhile, the first pads 4114A are welded with the ground terminals (not shown) of the temperature sensors 414, and the two second pads 4114B are respectively welded with the corresponding signal terminals (not shown) of the two temperature sensors 414, thereby the ground terminals (not shown) of the two temperature sensors 414 are both electrically connected with the second conductive trace L2-30 of the main body part 4111, and the signal terminals (not shown) of the two temperature sensors 414 are electrically connected with the third conductive traces L3-30, L3′-30 of the main body part 4111, respectively. That is, the two temperature sensors 414 transmit the temperature signals monitored thereby in parallel through the second conductive trace L2-30 and the third conductive traces L3-30, L3′-30. The two temperature sensors 414 are welded on the main body part 4111 and are respectively accommodated in the corresponding perforations 4131 of the dielectric element 413. Preferably, the temperature sensor 414 is a thermistor.

The wiring part 4112 has the same structure as the main body part 4111 and includes a corresponding insulating substrate B-30 and four paths of the conductive traces L-30 embedded in the insulating substrate B-30. The four paths of the conductive traces L-30 of the wiring part 4112 are electrically connected to the corresponding conductive traces L-30 of the main body part 4111 in one-to-one correspondence, respectively. The four golden fingers 41120 of the wiring part 4112 are all exposed from the side of the insulating substrate B-30 close to the dielectric element 413. The four paths of the conductive traces L-30 of the wiring part 4112 are electrically connected with the golden fingers 41120, respectively. The four paths of the conductive traces L-30 of the wiring part 4112 are also the first conductive trace L1-30, the second conductive trace L2-30, and the third conductive traces L3-30 and L3-30′, respectively. The first conductive trace L1-30 of the wiring part 4112 is extended from the first conductive trace L1-30 of the main body part 4111. The second conductive trace L2-30 of the wiring part 4112 is extended from the second conductive trace L2-30 of the main body part 4111. The third conductive traces L3-30, L3-30′ of the wiring part 4112 are respectively extended from the corresponding third conductive traces L3-30, L3-30′ of the main body part 4111.

The wiring part 4112 realizes the electrical connection with the conductive pad 4113 of the main body part 4111 by connecting the first conductive trace L1-30 thereof with the first conductive trace L1-30 of the main body part 4111, and connecting the first conductive trace L1-30 of the main body part 4111 with the conductive pads 4113 on the main body part 4111, and further realizes the electrical connection with the dielectric element 413 through the welding of the conductive pad 4113 of the main body part 4111 and the dielectric element 413. The wiring part 4112 realizes the electrical connection with the first pads 4114A of the main body part 4111 by connecting the second conductive trace L2-30 thereof with the second conductive trace L2-30 of the main body part 4111, and connecting the second conductive trace L2-30 of the main body part 4111 with the first pads 4114A on the main body part 4111, and further realizes the electrical connection with the ground terminals (not shown) of the temperature sensors 414 through the welding of the first pads 4114A and the temperature sensors 414. The wiring part 4112 realizes the electrical connection with the two second pads 4114B of the main body part 4111 by respectively connecting the third conductive traces L3-30, L3-30′ thereof with the corresponding third conductive traces L3-30, L3-30′ of the main body part 4111, and respectively connecting the third conductive traces L3-30, L3-30′ of the main body part 4111 with the corresponding second pads 4114B, and further realizes the parallel electrical connection with the signal terminals (not shown) of the two temperature sensors 414 by respectively welding the two second pads 4114B with the corresponding signal terminals (not shown) of the two temperature sensors 414. Thus, the temperature signals monitored by the two temperature sensors can be rapidly transmitted to the electric field generator (not shown) in parallel, so that the electric field generator (not shown) may timely and efficiently adjust the alternating voltage or alternating current of the alternating electrical signals applied to the dielectric element 413 to avoid cryogenic burns caused by excessively high temperature.

The main body part 4111 and the wiring part 4112 together constitute the flexible circuit board 411 of the electrical functional component 41. The respective insulating substrates B-30 of the wiring part 4112 and the main body part 4111 together constitute the insulating substrate B-30 of the flexible circuit board 411. The conductive trace L-30 of the main body part 4111 and the conductive trace L-30 of the wiring part 4112 are arranged in one-to-one correspondence and constitute the conductive trace L-30 of the flexible circuit board 411.

In view of the formation of the electrode unit 410, the insulating plate 412 is arranged on the side of the main body part 4111 of the flexible circuit board 411 away from the human skin, the dielectric element 413 is arranged on the side of the main body part 4111 of the flexible circuit board 411 facing the human skin, and the two temperature sensors 414 are arranged on the side of the main body part 4111 of the flexible circuit board 411 facing the human skin. The insulating plate 412 and the dielectric element 413 are respectively arranged on opposite sides of the main body part 4111 of the flexible circuit board 411. The first conductive trace L1-30 of the flexible circuit board 411 connects the six spaced conductive cores 41130 of the conductive pad 4113 in series. The second conductive trace L2-30 is electrically connected to the ground terminals (not shown) of two temperature sensors 414, respectively, through two first pads 4114A. The third conductive traces L3-30, L3-30′ are electrically connected to the signal terminals (not shown) of two temperature sensors 414, respectively, through the two second pads 4114B. The first conductive trace L1-30 is located in the insulating substrate B-30 at a layer close to the human skin. The second conductive trace L2-30 and the third conductive traces L3-30, L3-30′ are located in the insulating substrate B-30 at a layer close to the insulating plate 412. In order to facilitate the laying of the conductive traces L-30, the width of the wiring part 4112 is 7 mm to 9 mm. Preferably, the width of the wiring part 4112 is 8 mm.

The golden fingers 41120 of the wiring part 4112, the six conductive cores 41130 of the conductive pad 4113, and the pads 4114 are all exposed from the side of the insulating substrate B-30 of the flexible circuit board 411 close to the dielectric element 413. The golden finger 41120, the six conductive cores 41130 of the conductive pad 4113, and the pad 4114 are all located on the side of the flexible circuit board 411 close to the patient's body surface. One golden finger 41120 of the wiring part 4112 has an end electrically connected with the dielectric element 413 through the first conductive trace L1-30 connected thereto and the other end welded with the corresponding part of the wire 45, so as to transmit the alternating electrical signal generated by the electric field generator (not shown) to the dielectric element 413. One of the other three golden fingers 41120 of the wiring part 4112 has an end electrically connected to the ground terminals (not shown) of the temperature sensors 414 through the second conductive trace L2-30 connected thereto, and the other two of the other three golden fingers 41120 have ends electrically connected to the signal terminals (not shown) of the temperature sensors 414 through the third conductive traces L3-30, L3-30′ connected thereto. The other ends of the other three golden fingers 41120 are respectively welded with the corresponding parts of the wire 45. Thus, it realizes that the related signals detected by the temperature sensors 414 are transmitted to the electric field generator (not shown) quickly and concurrently through the second conductive trace L2-30, the third conductive traces L3-30, L3-30′ and the wire 45. Therefore, the alternating voltage or alternating current of the alternating electrical signals applied to the dielectric element 413 may be changed in time and quickly by the electric field generator (not shown) to achieve the purpose of avoiding cryogenic burns.

An alternate implementation of the second embodiment of the insulated electrode

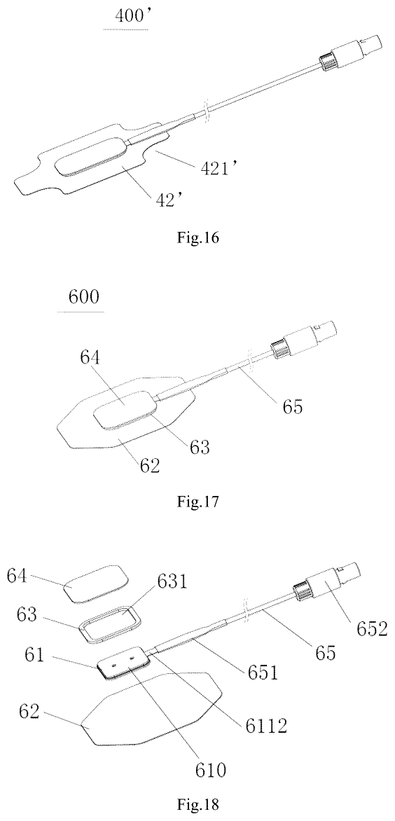

Referring to FIG. 16, the insulated electrode 400′ is an alternate embodiment of the insulated electrode 400 of the second embodiment. The only difference between the insulated electrode 400′ and the insulated electrode 400 is that: the four corners of the backing 42′ of the insulated electrode 400′ are provided with concave corners 421′ recessed inward. The backing 42′ is substantially arranged in a cross-shaped configuration. The concave corners 421′ communicate with the outside, and are in an “L” shape. When the insulated electrode 400′ is attached to the body surface corresponding to the patient's tumor site, the concave corner 421′ 5 may prevent the corners of the backing 42′ from arching and forming wrinkles, and further avoid air entering between the electrode unit 410 and the skin from the wrinkles, which results in cryogenic burns due to the increase of heat generation of the electrical functional component 41 causing by the increase of the impedance between the electrical functional component 41 and the skin.

Since the insulated electrodes 400, 400′ of the present embodiment use a separate electrode unit 410, it is easy to replace, and can be freely combined according to the size of the patient's tumor site, ensuring the effect of treating fields therapy. Meanwhile, the flexible circuit board 411 of the insulated electrodes 400, 400′ of the present disclosure is only provided with: a path of the first conductive trace L1-30 electrically connected with the dielectric element 413; a path of the second conductive trace L2-30 electrically connected with both the ground terminals (not shown) of the two temperature sensors 414; and two paths of the third conductive traces L3-30, L3-30′ electrically connected with the signal terminals (not shown) of the two temperature sensors 414 respectively, which realizes to transmit the alternating electrical signal of the electric field generator (not shown) to the dielectric element 413 through the first conductive trace L1-30, and realizes the purpose of applying the alternating electrical signal to the patient's tumor site for tumor treatment. At the same time, it realizes the signal transmission between the electric field generator (not shown) and the two temperature sensors 414 by electrically connecting the second conductive trace L2-30 and the third conductive trace L3-30, L3-30′ with the two temperature sensors 414, respectively. Thus, the difficulty of layering design is low; the structure is simple, the manufacturing process is simplified, the manufacturing is easy, and the product manufacturing yield is high, which may greatly reduce the manufacturing cost.

Furthermore, since the insulated electrodes 400, 400′ use a separate electrode unit 410 to apply an alternating electrical signal to the patient's tumor site, when it doesn't work properly, it only needs to replace the insulated electrodes 400, 400′ with the separate electrode unit 410 without needing to scrap the entire insulated electrode containing a plurality of electrode units 410, which can reduce the cost of tumor treatment for a patient. In addition, the insulated electrodes 400, 400′ of the present embodiment may be freely combined in quantity according to the patient's tumor site and the size of the patient's tumor site, ensuring the coverage area of the insulated electrodes 400, 400′ for tumor treating fields therapy and ensuring the required electric field strength of the tumor treating fields therapy. Moreover, the relative positions of the plurality of insulated electrodes 400, 400′ may also be freely adjusted according to the patient's own physical differences, tumor site, and tumor size, in order to obtain the optimal electric field strength and coverage area for tumor treatment. At the same time, it may allow the skin on the patient's body surface to which the insulated electrodes 400, 400′ are attached to breathe freely, avoiding the accumulation of heat on the patient's body surface caused by a long-term tumor treating fields therapy, and being not able to dissipate in time, which causes sweating and clogging pores and results in skin inflammation.

A third embodiment of the insulated electrode

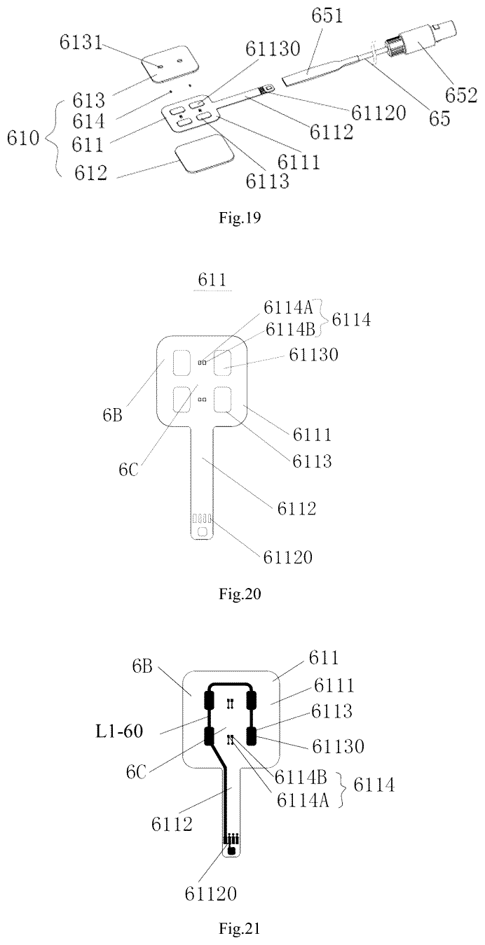

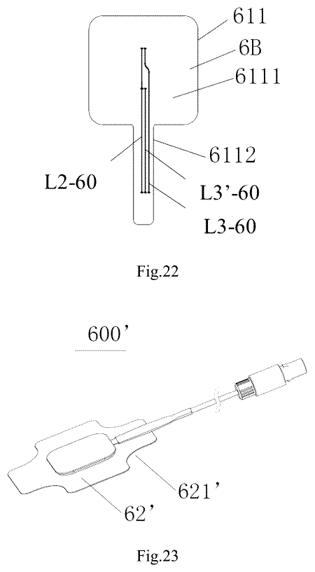

Referring to FIG. 17 to FIG. 22, a plurality of insulated electrodes 600 of the present embodiment may be used in combination, and the plurality of insulated electrodes 600 are connected with an adapter (not shown) to jointly perform tumor treating fields therapy on a tumor site. The insulated electrode 600 includes: a backing 62: an electrical functional component 61 adhered to the backing 62: a support member 63 adhered to the backing 62: an adhesive 66 that covers the corresponding parts of the electrical functional component 61 and the support member 63 and adheres to the body surface skin corresponding to the patient's tumor site; and a wire 65 electrically connected to the electrical functional component 61. The insulated electrode 600 is attached to the body surface corresponding to a patient's tumor site through the backing 62, and an alternating electric field is applied to the patient's tumor site through the electrical functional component 61 to interfere or prevent the mitosis of the patient's tumor cells, so as to achieve the purpose of treating the tumor.

The electrical functional component 61 includes a single electrode unit 610 in a square sheet shape and a wiring part 6112 connected with the electrode unit 610. The wiring part 6112 is welded with the wire 65 to realize the electrical connection between the electrical functional component 61 and the wire 65. A plurality of golden fingers 61120 are arranged on the surface of the wiring part 6112. In the present embodiment, there are four golden fingers 61120 arranged on the surface of the wiring part 6112 facing the skin. The wire 65 is welded with the golden fingers 61120 of the wiring part 6112, and the periphery of the welding point is covered with a heat shrinkable sleeve 651. The heat shrinkable sleeve 651 provides support and insulation protection for the connecting point between the wire 65 and the wiring part 6112 of the electrical functional component 61, so as to prevent the connecting point between the wire 65 and the wiring part 6112 of the electrical functional component 61 from being broken. Meanwhile, it may also be dustproof and waterproof. The end of the wire 65 away from the wiring part 6112 is configured with a first plug 652 electrically connected to an electric field generator (not shown) or an adapter (not shown). One end of the wire 65 is electrically connected to the golden fingers 61120 of the wiring part 6112, and the other end of the wire 65 is electrically connected with the electric field generator (not shown) or the adapter (not shown) through the first plug 652, so as to provide the insulated electrode 600 with alternating current signals for tumor treatment during the tumor treating fields therapy.

The electrode unit 610 includes: a main body part 6111: an insulating plate 612 arranged at one side of the main body part 6111 away from the human skin: a dielectric element 613 arranged at one side of the main body part 6111 facing the human skin; and two temperature sensors 614 arranged on the main body part 6111 and located at the same side as the dielectric element 613. The main body part 6111, the insulating plate 612 and the dielectric element 613 are almost the same of shapes and are all in the structure of a square sheet. The main body part 6111, the insulating plate 612, and the dielectric element 613 are correspondingly arranged along the thickness direction of the main body part 6111 and have centers located on the same straight line. In the present embodiment, the main body part 6111, the insulating plate 612 and the dielectric element 613 are all in the structure of a square sheet with arc-shaped corners. Preferably, the main body part 6111 is in the structure of a square sheet with a dimension of approximately 32 mm×32 mm. The wiring part 6112 of the electrical function component 61 is laterally extended from the main body part 6111 of the electrode unit 610.

The main body part 6111 includes an insulating substrate 6B and four paths of conductive traces L-60 embedded in the insulating substrate 6B. The four paths of conductive traces include: one path of a first conductive trace L1-60 arranged on a side of the insulating substrate 6B close to the dielectric element 613, one path of a second conductive trace L2-60 arranged on a side of the insulating substrate B close to the insulating plate 612, and two paths of third conductive traces L3-60 and L3′-60 arranged on the same side as the second conductive trace L2-60. The main body part 6111 is centrally provided with a conductive pad 6113 that is exposed from the insulating substrate 6B and is electorally connected with the first conductive trace L1-60. The conductive pad 6113 may be welded with the dielectric element 613 to assemble the dielectric element 6113 on the main body part 6111. The conductive pad 6113 may be completely covered by the dielectric element 613, so that the conductive pad 6113 and the dielectric element 613 can be welded by solder (not shown). The center of the conductive pad 6113 is located on the center line of the main body part 6111. The conductive pad 6113 includes a plurality of conductive cores 61130 center-symmetrically arranged, which may effectively prevent the offset of the position of the dielectric element 613 caused by the stacking of the solder (not shown) during the welding process. The top surfaces of the plurality of conductive cores 61130 are located on the same plane, which may avoid pseudo soldering when welding with the dielectric element 613. The plurality of conductive cores 61130 are all connected with a first conductive trace L1-60. The plurality of conductive cores 61130 are connected in series by the first conductive trace L1-60.

In the present embodiment, the conductive pad 6113 of the main body part 6111 is substantially in a square structure, and has symmetry axes respectively coincident with the symmetry axes of the main body part 6111. The conductive pad 6113 includes four spaced conductive cores 61130 located at four corners thereof. The conductive cores 61130 are arranged in a multi-point interval mode, so that the consumption of cop foil for manufacturing the conductive core 61130 can be reduced. At the same time, the amount of solder (not shown) used for welding the conductive cores 61130 and the dielectric element 613 can be saved, reducing the manufacturing cost. Each conductive core 61130 is in a rectangular structure with a dimension of approximately 8 mm×4 mm. Preferably, each conductive core 61130 is constructed in a rectangular shape with rounded corners. The longitudinal axis of each conductive core 61130 is parallel to the extension direction of the wiring part 6112. In other embodiments, each conductive core 61130 of the conductive pad 6113 may also be circular, square, etc.

In the present embodiment, the four conductive cores 61130 constituting the conductive pad 6113 are arranged in a matrix, and the four conductive cores 61130 are arranged in two rows and two columns. The gap between two columns of the conductive cores 61130 is about 8.5 mm, and the gap between two rows of the conductive cores 61130 is about 4 mm. The four conductive cores 61130 constituting the conductive pad 6113 are both center-symmetrically arranged and axial-symmetrically arranged, and each conductive core 61130 is also axial-symmetrically arranged, so that when the four conductive cores 61130 of the main body part 6111 are welded with the dielectric element 613, the stress at each welding point is balanced, ensuring an overall welding balance of the dielectric element 613 and improving the welding quality. Therefore, it may avoid an inclination of the dielectric element 613 caused by the unbalanced welding stress, which may result in a weak strength and easy fracture of the welding point on a side with larger spacing between the dielectric element 613 and the main body part 6111. At the same time, it may also avoid affecting the fit of the insulated electrode 600. Every two of the four conductive cores 61130 of the conductive pad 6113 are arranged at intervals, and a spacing 6C is formed between two adjacent conductive cores 61130. The four spacings 6C are communicated and substantially arranged in a cross shape. Adjacent spacings 6C are communicated. An extension direction of two of the four spacings 6C located between two conductive cores 61130 in the same row is consistent with the extension direction of the wiring part 6112.

The main body part 6111 is further provided with two pairs of pads 6114 that are exposed from the insulating substrate 6B, and the two pairs of pads 6114 are respectively welded with corresponding parts of the two corresponding temperature sensors 614, so as to realize the electrical connection between the temperature sensors 614 and the main body part 6111. Each pair of pads 6114 is located between the corresponding two conductive cores 61130 arranged at intervals in the same row: The two pairs of pads 6114 are both located in the extension direction of the wiring part 6112. Each pair of pads 6114 has a symmetrical center, and the line connecting the two symmetrical centers of the two pairs of pads 6114 is parallel to the extension direction of the wiring part 6112. Each pair of pads 6114 includes a first pad 6114A and a second pad 6114B. The first pads 6114A of the pairs of pads 6114 are electrically connected to the second conductive trace L2-60. One of the two second pads 6114B is electrically connected to the third conductive trace L3-60, and the other one of the two second pads 6114B is electrically connected to the third conductive trace L3′-60. Each temperature sensor 614 has a signal terminal (not shown) and a ground terminal (not shown). The first pad 6114A is welded with the ground terminal (not shown) of the temperature sensor 614, and the second pad 6114B is welded with the signal terminal (not shown) of the corresponding temperature sensor 614.

The insulating plate 612 is made of an insulating material. Preferably, the insulating plate 612 is made of an epoxy glass cloth laminate plate. The insulating plate 612 is adhered to a surface of the main body part 6111 away from human skin by sealant (not shown), which can enhance the strength of the main body part 6111, provide a flat welding plane for the welding operation between the main body part 6111 and the dielectric element 613, and improve the product yield rate. At the same time, the insulating plate 612 may also isolate the water vapor in the air on the side of the insulated electrode 600 away from the skin from contacting the solder (not shown) located between the main body part 6111 and the dielectric element 613, so as to prevent the water vapor from eroding the solder (not shown) between the main body part 6111 and the dielectric element 613, which may affect the electrical connection between the main body part 6111 and the dielectric element 613.

The dimension of the insulating plate 612 is the same as the dimension of the main body part 6111, so as to prevent the sealant (not shown) from climbing to the side of the main body part 6111 facing the human skin due to capillary effect when the insulating plate 612 is adhered to the side of the main body part 6111 away from the human skin by sealant (not shown), which may affect the filling of the sealant (not shown) in a gap (not shown) formed by welding the dielectric element 613 and the main body part 6111, resulting the existence of cavities in the sealant (not shown). This may further prevent the sealant (not shown) from bursting due to the rapid expansion of water vapor caused by a large difference in thermal expansion coefficients between the sealant (not shown) and the water vapor in the cavities during high-temperature curing, which may lead to popcorn phenomenon and damage the products.

The dielectric element 613 is made of a material with a high dielectric constant, and has conductive characteristics of blocking the conduction of direct current and allowing alternating current to pass through, which may ensure human safety. Preferably, the dielectric element 613 is made of a dielectric ceramic sheet. The dielectric element 613 is provided with two perforations 631 extended therethrough for accommodating the corresponding temperature sensors 614 respectively, in which the number of the perforations 631 is the same as the number of the temperature sensors 614. A metal layer (not shown) is attached to the surface of the dielectric element 613 facing the main body part 6111. A point-to-side welding is formed between the metal layer (not shown) of the dielectric element 613 and the conductive cores 61130 of the conductive pad 6113 of the main body part 6111, so that it is more convenient to weld without requiring higher welding alignment accuracy. The inner edge of the metal layer (not shown) of the dielectric element 613 and the edge of the perforation 631 of the dielectric element 613 are arranged at intervals, so as to prevent the solder (not shown) between the metal layer (not shown) of the dielectric element 613 and the main body part 6111 from spreading in a direction toward the perforation 631 of the dielectric element 613 when heated and melted, which may result in a short circuit of the temperature sensor 614. The outer edge of the metal layer (not shown) of the dielectric element 613 and the outer edge of the dielectric element 613 are also arranged at intervals, so as to prevent the solder (not shown) between the metal layer (not shown) of the dielectric element 613 and the main body part 6111 from overflowing to the outside of the main body part 6111 when heated and melted, which may result in a direct current that is not hindered by the dielectric element 613 passes through and acts on the patient's body surface when the insulated electrode 600 is attached to the body surface of the patient's tumor site.

Each temperature sensor 614 is welded with the first pad 6114A arranged on the main body part 6111 through the ground terminal (not shown) thereof, and is weld with the second pad 6114B arranged on the main body part 6111 through the signal terminal (not shown) thereof, so as to realize the electrical connection with the main body part 6111. The two first pads 6114A of the main body part 6111 are both electrically connected with the second conductive trace L2-60, one of the two second pads 6114B is electrically connected with the third conductive trace L3-60, the other of the two second pads 6114B is connected with the third conductive trace L3′-60, and the two first pads 6114A are respectively welded with the corresponding ground terminals (not shown) of the two temperature sensors 614, the two second pads 6114B are respectively welded with the corresponding signal terminals (not shown) of the two temperature sensors 614, thereby the ground terminals (not shown) of the two temperature sensors 614 are both electrically connected with the second conductive trace L2-60 of the main body part 6111, and the signal terminals (not shown) of the two temperature sensors 614 are electrically connected with the third conductive traces L3-60, L3′-60 of the main body part 6111, respectively. That is, the two temperature sensors 614 transmit the monitored temperature signals in parallel through the second conductive trace L2-60 and the third conductive traces L3-60, L3′-60. The two temperature sensors 614 are welded on the main body part 6111 and are respectively accommodated in the corresponding perforations 631 of the dielectric element 613. Preferably, the temperature sensor 614 is a thermistor. The temperature sensor 614 is used to monitor the temperature of the adhesive 64 covering the surface of the dielectric element 616 of the electrical functional component 61 facing the human skin, and further detect the temperature of the human skin to which the adhesive 64 is attached. When the temperature monitored by the temperature sensor 614 exceeds the upper limit of the safe temperature of human body, the tumor treating fields system (not shown) may reduce the alternating voltage or the alternating current of the alternating electrical signals or turn off the alternating electrical signals transmitted to the insulated electrode 600 in time, so as to avoid cryogenic burns of human body: The two temperature sensors 614 are symmetrically arranged on the main body part 6111, and may detect the temperatures of human skin corresponding to different positions and ensure the reliability of detection data. The two temperature sensors 614 are welded to the main body part 6111 through the two pairs of pads 6114 of the main body part 6111, and are then sealed with sealant (not shown), so as to prevent the water vapor from eroding the temperature sensors 614 and causing a failure of the temperature sensors 614.

The wiring part 6112 has the same structure as the main body part 6111, and also has a corresponding insulating substrate 6B and four paths of the conductive traces L-60 embedded in the insulating substrate 6B. The four paths of the conductive traces L-60 of the wiring part 6112 are respectively electrically connected to the corresponding conductive traces L-60 of the main body part 6111 in one-to-one correspondence. The four golden fingers 61120 of the wiring part 6112 are all exposed from the side of the insulating substrate 6B close to the dielectric element 613. The four paths of the conductive traces L-60 of the wiring part 6112 are electrically connected with the golden fingers 61120, respectively. The four paths of the conductive traces L-60 of the wiring part 6112 are also the first conductive trace L1-60, the second conductive trace L2-60, and the third conductive traces L3-60 and L3-60′, respectively. The first conductive trace L1-60 of the wiring part 6112 is extended from the first conductive trace L1-60 of the main body part 6111. The second conductive trace L2-60 of the wiring part 6112 is extended from the second conductive trace L2-60 of the main body part 6111. The third conductive traces L3-60, L3-60′ of the wiring part 6112 are respectively extended from the corresponding third conductive traces L3-60, L3-60′ of the main body part 6111.

The wiring part 6112 realizes the electrical connection with the conductive pad 6113 of the main body part 6111 by connecting the first conductive trace L1-60 thereof with the first conductive trace L1-60 of the main body part 6111, and connecting the first conductive trace L1-60 of the main body part 6111 with the conductive pad 6113 on the main body part 6111, and further realizes the electrical connection with the dielectric element 613 by welding the conductive pad 6113 of the main body part 6111 with the dielectric element 613. The wiring 20 part 6112 realizes the electrical connection with the first pad 6114A of the main body part 6111 by connecting the second conductive trace L2-60 thereof with the second conductive trace L2-60 of the main body part 6111, and connecting the second conductive trace L2-60 of the main body part 6111 with the first pads 6114A on the main body part 6111, and further realizes the electrical connection with the ground terminals (not shown) of the temperature sensors 614 by welding the first pads 6114A with the temperature sensors 614. The wiring part 6112 realizes the electrical connection with the two second pads 6114B of the main body part 6111 through a corresponding connection of the third conductive traces L3-60, L3-60′ thereof respectively with the third conductive traces L3-60, L3-60′ of the main body part 6111, and the connection of the third conductive traces L3-60, L3-60′ of the main body part 6111 respectively with the two second pads 6114B, and further realizes the electrical connection with the signal terminals (not shown) of the two temperature sensors 614 through a welding in one-to-one correspondence between the two second pads 6114B and the signal terminals (not shown) of the two temperature sensors 614. Thus, the temperature signals monitored by the temperature sensors 614 may be transmitted to the electric field generator (not shown) in parallel, so that the electric field generator (not shown) may timely and efficiently adjust the alternating voltage or alternating current of the alternating electrical signal applied to dielectric element 613 to avoid cryogenic burns caused by excessively high temperature.

The main body part 6111 and the wiring part 6112 together constitute the flexible circuit board 611 of the electrical functional component 61. The respective insulating substrates 6B of the wiring part 6112 and the main body part 6111 together constitute the insulating substrate 6B of the flexible circuit board 611. The conductive trace L-60 of the main body part 6111 and the conductive trace L-60 of the wiring part 6112 are arranged in one-to-one correspondence and constitute the conductive trace L-60 of the flexible circuit board 611. The insulating substrate 6B of the flexible circuit board 611 may isolate the water vapor in the air around the insulated electrode 600 from the solder (not shown) located between the conductive pad 6113 and the dielectric element 613, and prevent the water vapor in the air away from the skin from eroding the solder (not shown) located between the dielectric element 613 and the flexible circuit board 611 with the conductive pad 6113 on the main body part 6111 of the flexible circuit board 611. The insulating substrate 6B of the flexible circuit board 611 and the insulating plate 612 function as a double isolation, which may prolong the service life of the insulated electrode 600.

In view of the formation of the electrode unit 610, the insulating plate 612 is arranged on the side of the main body part 6111 of the flexible circuit board 611 away from the human skin, and the dielectric element 613 is arranged on the side of the main body part 6111 of the flexible circuit board 611 facing the human skin. The two temperature sensors 614 are arranged on the side of the main body part 6111 of the flexible circuit board 611 facing the human skin. The insulating plate 612 and the dielectric element 613 are respectively arranged on opposite sides of the main body part 6111 of the flexible circuit board 611. The first conductive trace L1-60 of the flexible circuit board 611 connects the four spaced conductive cores 61130 of the conductive pad 6113 in series. The second conductive trace L2-60 is electrically connected to the ground terminals (not shown) of two temperature sensors 614 through two first pads 6114A. The third conductive traces L3-60, L3-60′ are electrically connected to the signal terminals (not shown) of two temperature sensors 614 through two second pads 6114B, respectively. The first conductive trace L1-60 is located in the insulating substrate 6B at a layer close to the human skin. The second conductive trace L2-60 and the third conductive traces L3-60, L3-60′ are located in the insulating substrate 6B at a layer close to the insulating plate 612. In order to facilitate the laying of the conductive traces L-60, the width of the wiring part 6112 is 7 mm to 9 mm. Preferably, the width of the wiring part 6112 is 8 mm.

The golden fingers 61120 of the wiring part 6112, four conductive cores 61130 of the conductive pad 6113, and the pads 6114 are all exposed from the side of the insulating substrate 6B of the flexible circuit board 611 close to the dielectric element 613. The golden fingers 61120, the four conductive cores 61130 of the conductive pad 6113, and the pads 6114 are all located on the side of the flexible circuit board 611 close to the patient's body surface. One golden finger 61120 of the wiring part 6112 has an end electrically connected with the dielectric element 613 through the first conductive trace L1-60 connected thereto and the other end welded with the corresponding part of the wire 65, so as to transmit the alternating electrical signal generated by the electric field generator (not shown) to the dielectric element 613. One of the other three golden fingers 61120 of the wiring part 6112 has an end electrically connected to the ground terminals (not shown) of the temperature sensors 614 through the second conductive trace L2-60 connected thereto, and the other two of the other three golden fingers 61120 have ends electrically connected to the signal terminals (not shown) of the two temperature sensors 614 through the third conductive traces L3-60, L3-60′ connected thereto. The other ends of the other three golden fingers 61120 of the wiring part 6112 are respectively welded with the corresponding parts of the wire 65. Thus, it realizes that the related signals detected by the temperature sensors 614 are transmitted in parallel to the electric field generator (not shown) through the second conductive trace L2-60, the third conductive traces L3-60, L3-60′ and the wire 65.

The backing 62 is in a sheet shape, which is mainly made of a flexible and permeable insulating material. The backing 62 is a mesh fabric. Specifically, the backing 62 is a mesh non-woven fabric, which has the characteristics of softness, lightness, moisture-proof and permeability, and can keep the patient's skin surface dry after being attached to the patient's body surface for a long time. The side of the backing 62 facing the patient's body surface is also coated with a biocompatible adhesive (not shown) for tightly adhering the backing 62 to the body surface corresponding to the patient's tumor site. In the present embodiment, the backing 62 is substantially in an octagonal sheet structure.

The support member 63 is adhered to the backing 62, and surrounds the outside of the electrode unit 610. In the middle of the support member 63, a through hole 631 is disposed therethrough for accommodating the electrode unit 610. The support member 63 may be made of foam material. The support member 63 is flush with the surface of a side of the electrode unit 610 away from the backing 62. That is, the support member 63 is flush with the surface of a side of the electrode unit 610 facing the adhesive 64 to support the adhesive 64.

The adhesive 64 has double-sided adhesiveness. One surface of the adhesive 64 is adhered to the surface of the support member 63 and the surface of a side of the electrode unit 310 away from the backing 62. The other surface of the adhesive 64 is used as an adhesive lay, which is attached to the skin of human body surface to keep the skin surface moist and relieve local pressure. Preferably, the adhesive 64 may use conductive hydrogel to serve as a conductive medium. With the support of the support member 63, the adhesive 64 has better adhesion to human skin.

The insulated electrode 600 of the present embodiment is substantially the same as the insulated electrode 400, and the only difference is that: the shape and dimension of the electrode unit 610 is different, and the shape, dimension, or arrangement of the two pairs of pads 6114 and the conductive pad 6113 correspondingly arranged on the main body part 6111 is different. The following only describes the differences, and other contents may refer to the insulated electrode 400 of the second embodiment.

The electrode unit 610 is in a square sheet shape, and the main body part 6111, the insulating plate 612, and the dielectric element 613 are all in the structure of a square sheet with arc-shaped corners. The dimension of the main body part 6111 is approximately 32 mm×32 mm. The conductive pad 6113 of the main body part 6111 is substantially in a square structure, and has symmetry axes coincident with the symmetry axes of the main body part 6111. The conductive pad 6113 includes four conductive cores 61130 located at four corners and arranged at intervals. Each conductive core 61130 is in the structure of a rectangular with a dimension of approximately 9 mm×6 mm. Preferably, each conductive core 61130 is constructed in a rectangular shape with rounded corners. The longitudinal axis of each conductive core 61130 is parallel to the extension direction of the wiring part 6112.

The four conductive cores 61130 constituting the conductive pad 6113 are in a matrix shape, and the four conductive cores 61130 are arranged in two rows and two columns. The gap between two columns of the conductive cores 61130 is about 8.5 mm, and the gap between two rows of the conductive cores 61130 is about 4 mm. The four conductive cores 61130 constituting the conductive pad 6113 are both center-symmetrically arranged and axial-symmetrically arranged, and each conductive core 61130 is also axial-symmetrically arranged, so that when the four conductive cores 61130 of the main body part 6111 are welded with the dielectric element 613, the stress balance of each welding point is guaranteed, improving the welding quality. Every two of the four conductive cores 61130 of the conductive pad 6113 are arranged at intervals, and a spacing C-30 is formed between two adjacent conductive cores 61130. The four spacings C-30 are communicated and substantially in a cross shape. Adjacent spacings C-30 are communicated. An extension direction of two of the four spacings C-30 located between two conductive cores 61130 in the same row is consistent with the extension direction of the wiring part 6112.

The two pairs of pads 6114 of the main body part 6111 are respectively located between the corresponding two conductive cores 61130 arranged at intervals in the row: The two pairs of pads 6114 are both located in the extension direction of the wiring part 6112. Each pair of pads 6114 has a symmetrical center, and the line connecting the two symmetrical centers of the two pairs of pads 6114 is parallel to the extension direction of the wiring part 6112.

An alternate implementation of the third embodiment of the insulated electrode

Referring to FIG. 23, the insulated electrode 600′ is an alternate embodiment of the insulated electrode 600 of the third embodiment. The only difference between the insulated electrode 600′ and the insulated electrode 600 is that: the four corners of the backing 62′ of the insulated electrode 600′ are provided as concave corners 621′ recessed inward. The backing 62′ is substantially in a cross-shaped configuration. The concave corners 621′ communicate with the outside, and are in an “L” shape. When the insulated electrode 600′ is attached to the body surface corresponding to the patient's tumor site, the concave corners 621′ may prevent the corners of the backing 62′ from arching and forming wrinkles, and further avoid air entering between the electrode unit 610 and the skin from the wrinkles, which results in cryogenic burns due to the increase of heat generation of the electrical functional component 61 causing by the increase of the impedance between the electrical functional component 61 and the skin.

Since the insulated electrodes 600, 600′ of the present embodiment use a separate electrode unit 610 to apply an alternating electrical signal to the patient's tumor site, when it doesn't work properly, it only needs to replace the insulated electrodes 600 with the separate electrode unit 610 without needing to scrap the entire insulated electrode containing a plurality of electrode units 610, which can reduce the cost of tumor treatment for a patient. In addition, the insulated electrodes 600, 600′ of the present embodiment may be freely combined in quantity according to the patient's tumor site and the size of the patient's tumor site, ensuring the coverage area of the insulated electrodes 600, 600′ for tumor treating fields therapy and ensuring the required electric field strength of the tumor treating fields therapy. Meanwhile, the flexible circuit board 611 of the insulated electrodes 600, 600′ of the present embodiment is only provided with: a path of the first conductive trace L1-60 electrically connected with the dielectric element 613: a path of the second conductive trace L2-60 electrically connected together with the ground terminals (not shown) of the two temperature sensors 614; and two paths of the third conductive traces L3-60, L3′-60 electrically connected with the signal terminals (not shown) of the two temperature sensors 614 respectively, which may realize to transmit the alternating voltage signal of the electric field generator (not shown) to the dielectric element 613 through the first conductive trace L1-60, and realize the purpose of applying the alternating electrical signal to the patient's tumor site for tumor treatment. At the same time, it realizes the signal transmission between the electric field generator (not shown) and the two temperature sensors 614 by electrically connecting the second conductive trace L2-60 and the third conductive trace L3-60, L3′-60 with the two temperature sensors 614, respectively. Thus, the difficulty of layering design is low, the structure is simple, the manufacturing process is simple, the manufacturing is easy; and the product manufacturing yield is high, which may greatly reduce the manufacturing cost.

Moreover, the relative positions of the plurality of insulated electrodes 600, 600′ may also be freely adjusted according to the patient's own physical differences, tumor site, and tumor size, in order to obtain the optimal electric field strength and coverage area for tumor treatment. At the same time, it may allow the skin on the patient's body surface to which the insulated electrodes 600, 600′ are attached to breathe freely, avoiding the accumulation of heat on the patient's body surface caused by a long-term tumor treating fields therapy, and being not able to dissipate in time, which causes sweating and clogging pores and results in skin inflammation.

A fourth embodiment of the insulated electrode

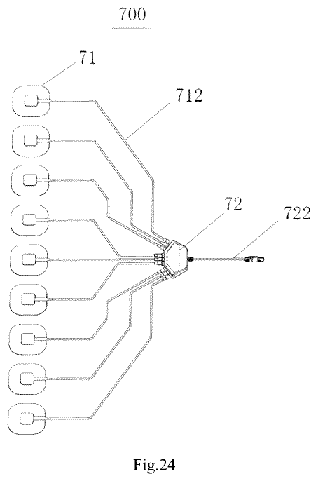

In the above three embodiments, the insulated electrode only has a single electrode unit, thus, during the treatment, a certain number of insulated electrodes are needed to ensure the therapeutic effect at the same time. However, for an electric field generator (not shown) or an adapter (not shown), the number of interfaces for connecting with insulated electrodes is limited. Therefore, the present application provides the following embodiments. Referring to FIG. 24 and FIG. 25, the insulated electrode 700 of the present embodiment includes: an electrical connector 72 electrically connected to an electric field generator (not shown) or an adapter (not shown); and a plurality of electrode sheets 71 detachably assembled on the electrical connector 72. The structure of the electrode sheet 71 may directly adopt the insulated electrodes 300, 400, 400′, 600, 600′ in the first to the third embodiments.

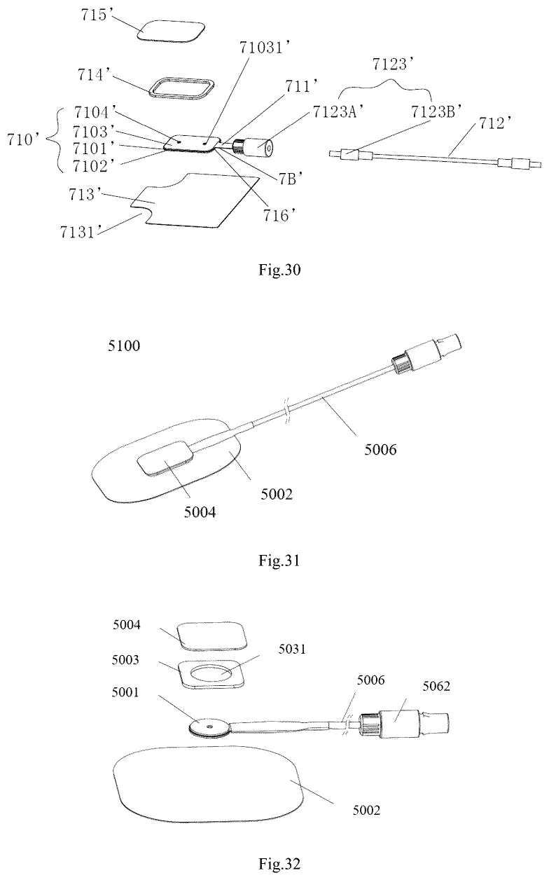

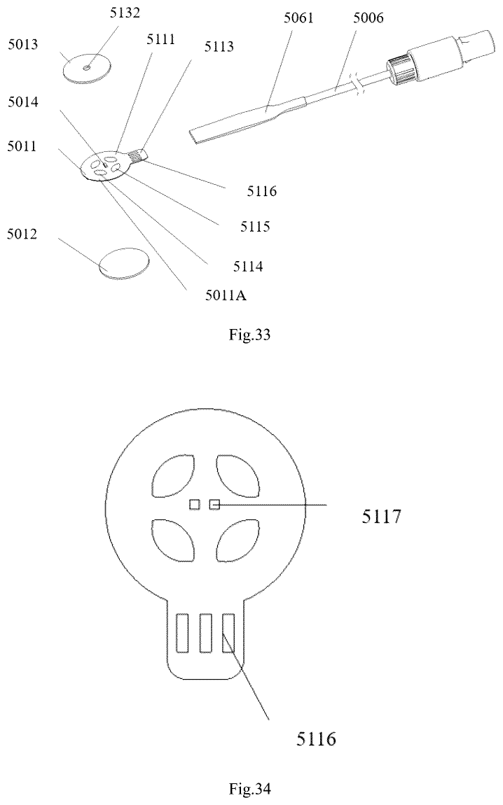

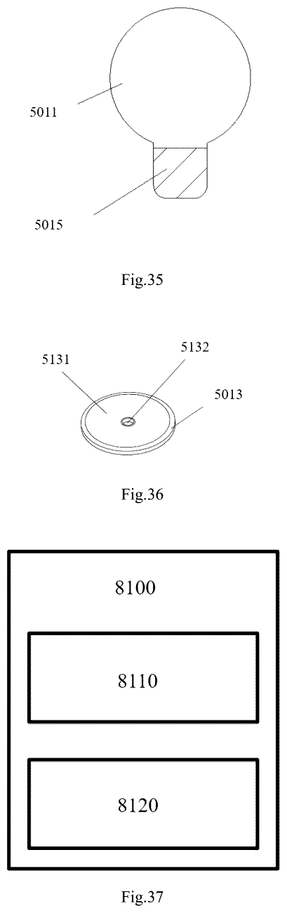

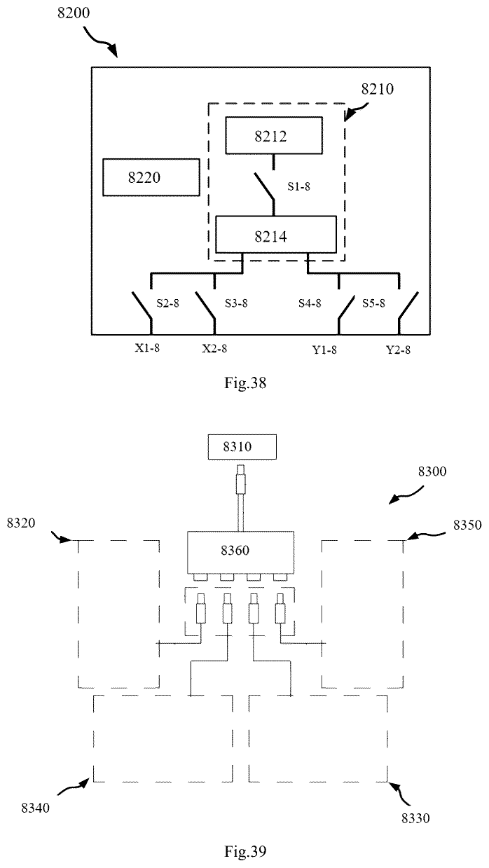

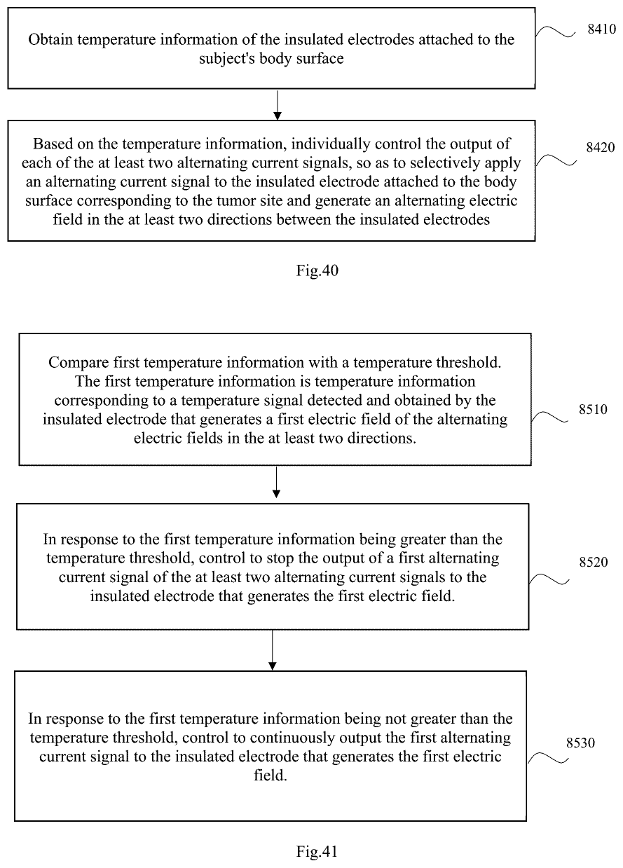

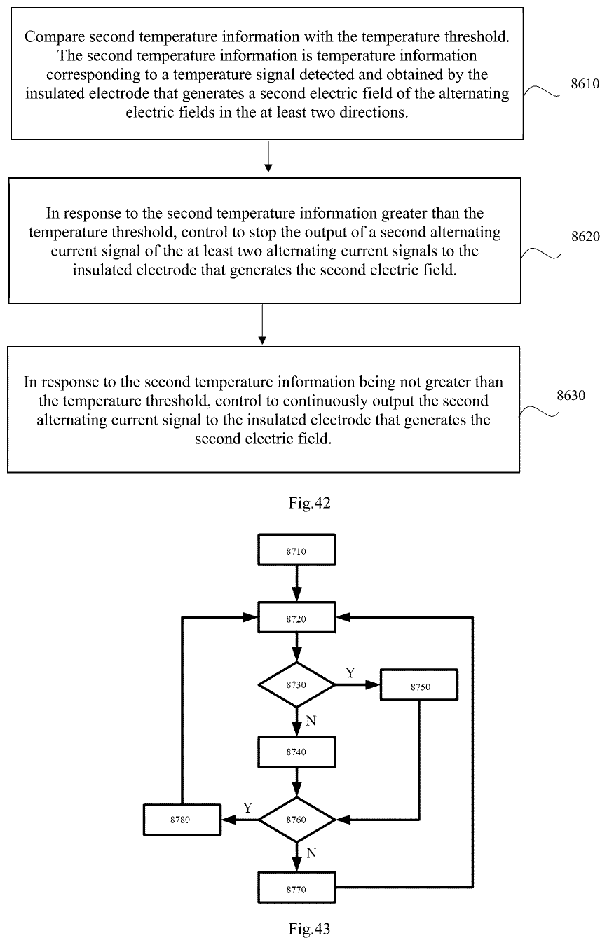

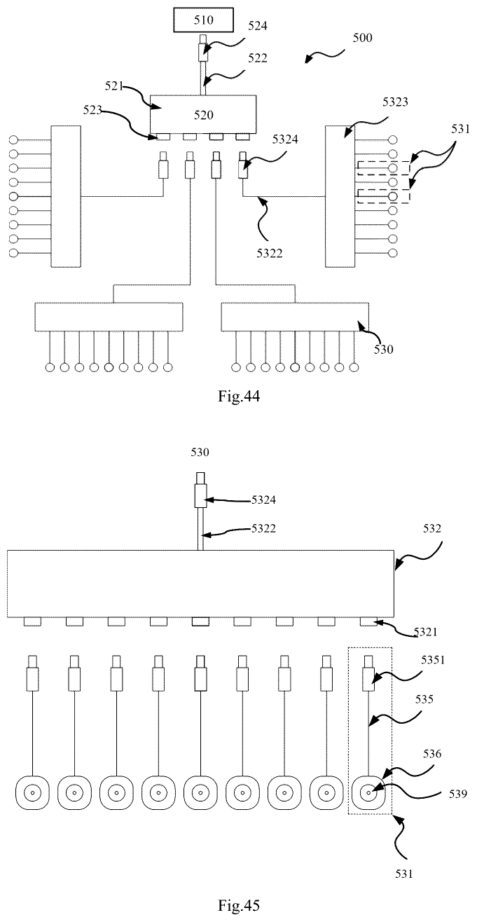

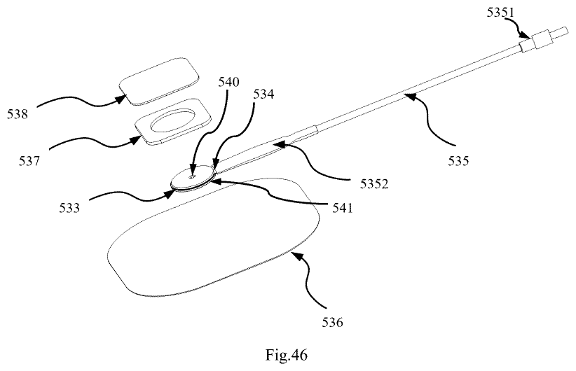

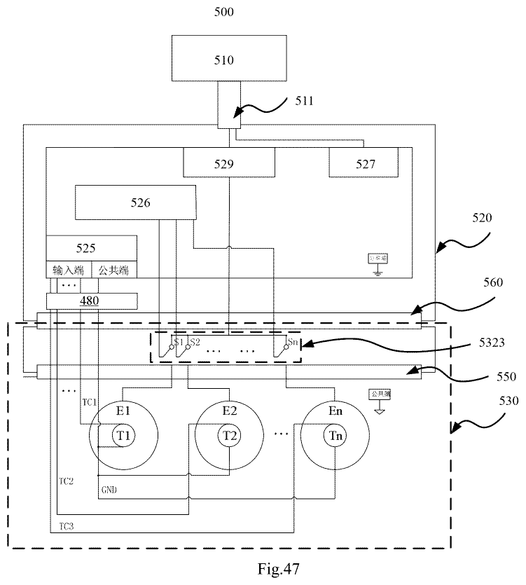

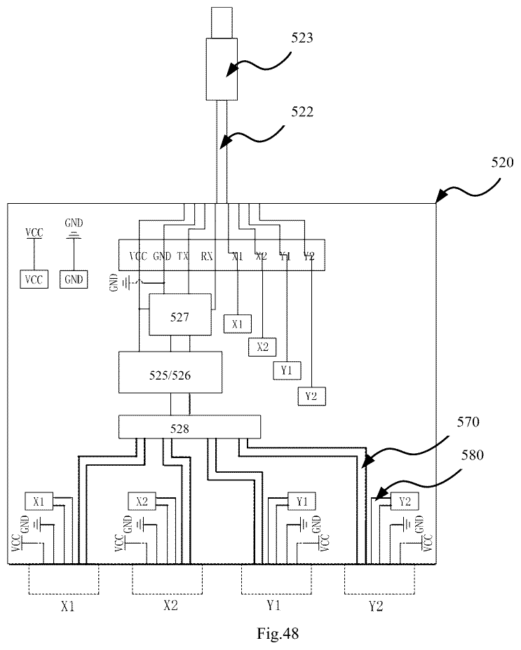

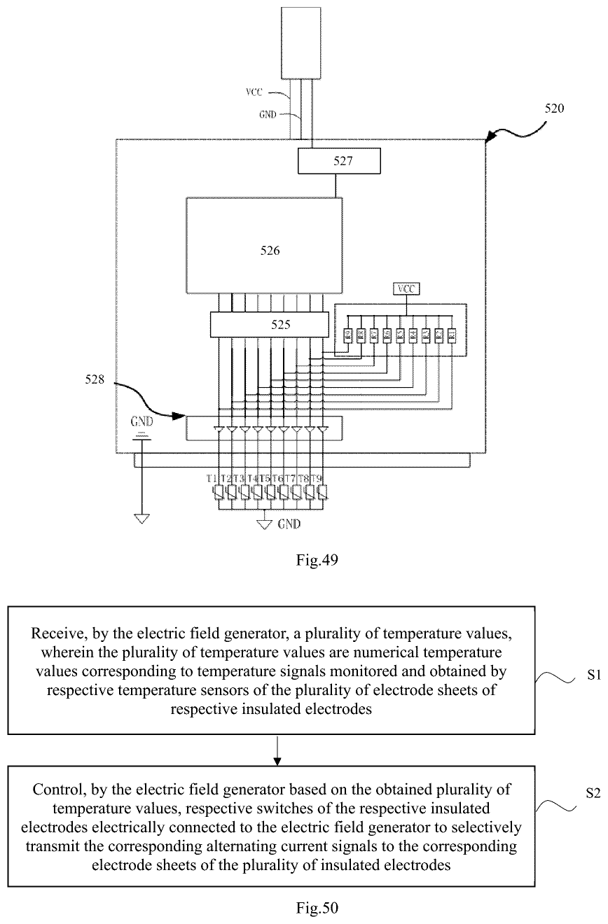

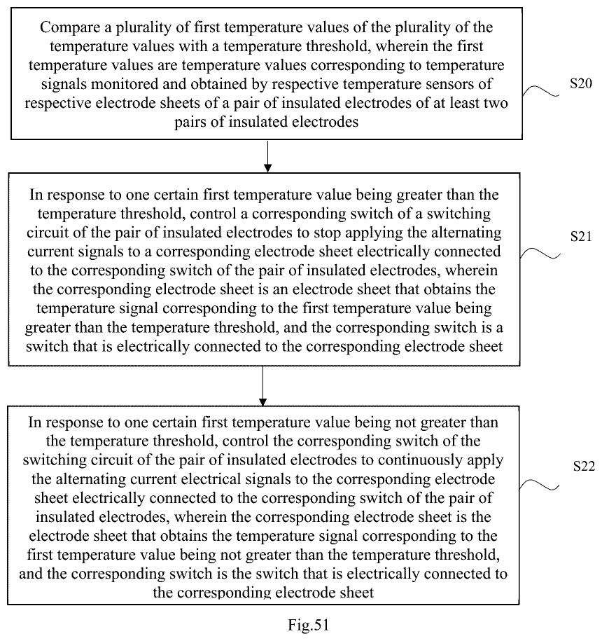

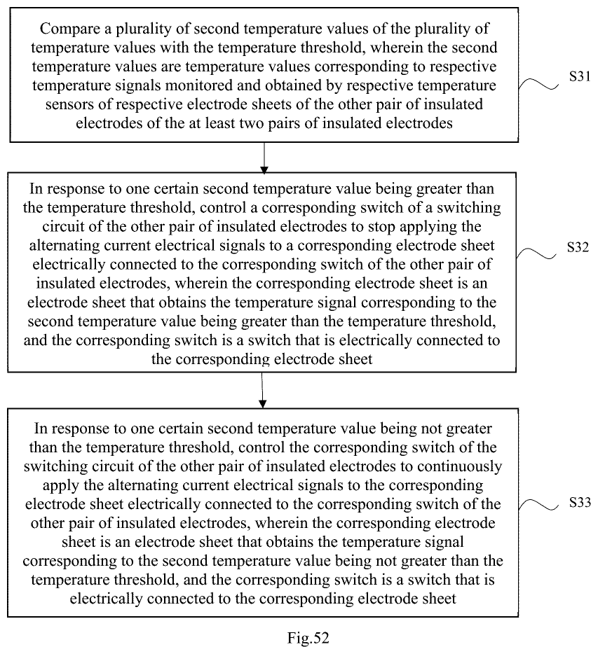

Mainly referring to FIG. 26 to FIG. 28, each electrode sheet 71 includes: a single electrode unit 710 for applying an alternating electric signal to a patient's tumor site: a wiring part 711 electrically connected to the electrode unit 710; a first wire 712 welded with the wiring part 711: a backing 713 adhered to the electrode unit 710; a support member 714 adhered to the backing 713 in a manner of surrounding the electrode unit 710; and an adhesive 715 covering the corresponding part of the support member 714 and the electrode unit 710. One end of the first wire 712 is welded with the wiring part 711, and the other end of the first wire 712 is detachably connected with the electrical connector 72 through a first plug 7121 arranged at the end of the first wire 712, so as to realize the electrical connection between the electrode unit 710 and the electrical connector 72, and thus the alternating current signals generated by the electric field generator (not shown) are transmitted to the electrode unit 710 through the electrical connector 72 for tumor treating fields therapy. Alternatively, the electrode sheet 71 may be directly plugged with an electric field generator (not shown) through the first plug 7121 of the first wire 712, or may be first plugged with an adapter (not shown) and then electrically connected with the electric field generator (not shown) through the adapter (not shown) to realize the electrical connection between the electrode sheet 71 and the electric field generator (not shown).

The electrode unit 710 of the electrode sheet 71 includes: a main body part 7101 arranged at the end of the wiring part 711 and electrically connected to the wiring part 711: an insulating plate 7102 and a dielectric element 7103 respectively arranged at two opposite sides of the main body part 7101; and a temperature sensor 7104 arranged on the main body part 7101 and located at the same side as the dielectric element 7103. The electrode unit 710 is in a circular sheet structure as a whole. The main body part 7101, the insulating plate 7102 and the dielectric element 7103 are all in the circular sheet structure, and are substantially the same in size and are arranged in one-to-one correspondence along the thickness direction. The centers of the main body part 7101, the insulating plate 7102 and the dielectric element 7103 are located on the same straight line.

The side of the main body part 7101 facing the human skin is provided with a conductive pad 7105, and the conductive pad 7105 is welded with the dielectric element 7103 to assemble the dielectric element 7103 on the main body part 7101. The conductive pad 7105 may be completely covered by the dielectric element 7103, so that the conductive pad 7105 and the dielectric element 7103 may be welded by a solder (not shown). The center of the conductive pad 7105 is located on the center line of the main body part 7101. The conductive pad 7105 includes a plurality of conductive cores 71051 center-symmetrically arranged, which may effectively prevent the offset of the position of the dielectric element 7103 caused by the stacking of the solder (not shown) during the welding process. The top surfaces of the plurality of conductive cores 71051 are located on the same plane, which may avoid pseudo soldering when welding with the dielectric element 7103. There is also a pair of pads 7106 arranged between the plurality of conductive cores 71051, which may be welded with the corresponding parts of the temperature sensor 7104 to realize the electrical connection between the temperature sensor 7104 and the main body part 7101. The two pads 7106 include a first pad 7106A and a second pad 7106B. The temperature sensor 7104 has a signal terminal (not shown) and a ground terminal (not shown). The first pad 7106A is welded with the ground terminal (not shown) of the temperature sensor 7104, and the second pad 7106B is welded with the signal terminal (not shown) of the temperature sensor 7104.

The insulating plate 7102 is made of an insulating material. Preferably, the insulating plate 7102 is made of an epoxy glass cloth laminate plate. The insulating plate 7102 is adhered to a surface of the main body part 7101 away from human skin by sealant (not shown). On one hand, it may enhance the strength of the main body part 7101 to provide a flat welding plane for the welding operation between the main body part 7101 and the dielectric element 7103. On the other hand, it may also isolate the water vapor in the air on the side of the electrode sheet 71 away from the skin from contacting the solder (not shown) located between the main body part 7101 and the dielectric element 7103, so as to prevent the water vapor from eroding the solder (not shown) between the main body part 7101 and the dielectric element 7103, which may affect the electrical connection between the main body part 7101 and the dielectric element 7103. The dimension of the insulating plate 7102 is substantively the same as the dimension of the main body part 7101, so as to prevent the sealant (not shown) from climbing to the side of the main body part 7101 facing the human skin by capillary effect when the insulating plate 7102 is adhered to a surface of the main body part 7101 away from human skin by sealant (not shown), which may affect the filling of the sealant (not shown) in a gap (not shown) formed by welding the dielectric element 7103 and the main body part 7101, resulting the existence of cavities in the sealant (not shown). This may further prevent the sealant (not shown) from bursting due to the rapid expansion of water vapor caused by a large difference in thermal expansion coefficients between the sealant (not shown) and the water vapor in the cavities during high-temperature curing, which may lead to popcorn phenomenon, and damage the products.

The dielectric element 7103 is made of a material with high dielectric constant, and has conductive characteristics of blocking the conduction of direct current and allowing alternating current to pass through, which may ensure user's safety during the tumor treating fields therapy. Preferably, the dielectric element 7103 is made of a dielectric ceramic sheet. The dielectric element 7103 is provided with a perforation 71031 extended through the middle thereof. The perforation 71031 is corresponding to the pair of pads 7106 of the main body part 7101, and is used for accommodating the corresponding temperature sensor 7104. A metal layer (not shown) is attached to the surface of the dielectric element 7103 facing the main body part 7101. A point-to-side welding is formed between the metal layer (not shown) of the dielectric element 7103 and the conductive cores 71051 of the conductive pad 7105 of the main body part 7101, so that it is more convenient to weld without requiring higher welding alignment accuracy. The inner edge of the metal layer (not shown) of the dielectric element 7103 and the edge of the perforation 71031 of the dielectric element 7103 are arranged at intervals, so as to prevent the solder (not shown) between the metal layer (not shown) of the dielectric element 7103 and the main body part 7101 from spreading in a direction toward the perforation 71031 of the dielectric element 7103 when heated and melted, which may result in a short circuit of the temperature sensor 7104. The outer edge of the metal layer (not shown) of the dielectric element 7103 and the outer edge of the dielectric element 7103 are also arranged at intervals, so as to prevent the solder (not shown) between the metal layer (not shown) of the dielectric element 7103 and the main body part 7101 from overflowing to the outside of the main body part 7101 when heated and melted, which may result in a direct current that is not hindered by the dielectric element 7103 passes through and acts on the patient's body surface when the electrode sheet 71 is attached to the body surface of the patient's tumor site.

The gap (not shown) formed by welding the dielectric element 7103 and the main body part 7101 is filled with sealant (not shown) to protect the solder (not shown) between the dielectric element 7103 and the main body part 7101, so as to prevent the dielectric element 7103 from being affected by external force, which leads to the fracture of the welding part, and further leads to the failure of the alternating electric field to be applied to the patient's tumor site through the dielectric element 7103. At the same time, it can also prevent the water vapor in the air from entering the gap (not shown) and eroding the solder (not shown) between the dielectric element 7103 and the main body part 7101, which may affect the electrical connection between the dielectric element 7103 and the main body part 7101. The outer diameter of the dielectric element 7103 is slightly smaller than the diameter of the main body part 7101. When filling the sealant (not shown), the sealant (not shown) can be filled into the gap (not shown) along the edge of the main body part 7101 located outside the dielectric element 7103 by capillary phenomenon, which is beneficial to the filling of the sealant (not shown) in the gap (not shown) formed by welding the dielectric element 7103 and the main body part 7101. When filling the sealant (not shown) in the gap (not shown) formed by welding the dielectric element 7103 and the main body part 7101, the air in the gap (not shown) may be exhausted from the perforation 71031 of the dielectric element 7103, so as to prevent the sealant (not shown) filled in the gap (not shown) from generating cavities, and thus improve the product quality.

The temperature sensor 7104 is arranged on the main body part 7101 by welding the ground terminal (not shown) thereof with the first pad 7106A arranged on the main body part 7101, and welding the signal terminal (not shown) thereof with the second pad 7106B arranged on the main body part 7101. The temperature sensor 7104 is welded on the main body part 7101 and is accommodated in the perforation 71031 of the dielectric element 7103. Preferably, the temperature sensor 7104 is a thermistor. The temperature sensor 7104 is used to monitor the temperature of the adhesive 715 covering the surface of the dielectric element 7103 of the electrode unit 710 facing the human skin, so as to further detect the temperature of the human skin to which the adhesive 715 is attached. When the temperature monitored by the temperature sensor 7104 exceeds the upper limit of the safe temperature of human body, the tumor treating fields system 3000 may reduce the alternating voltage of the alternating current of the alternating electrical signal or turn off the alternating electrical signal applied to the electrode sheet 71 by the electric field generator (not shown) in time, so as to avoid cryogenic burns of human body. The temperature sensor 7104 is welded to the main body part 7101 through the pair of pads 7106 of the main body part 7101, and is then sealed with sealant (not shown), so as to prevent the water vapor from eroding the temperature sensor 7104 and causing a failure of the temperature sensor 7104.

The wiring part 711 is laterally extended from the main body part 7101 of the electrode unit 710. The periphery of the welding point of the wiring part 711 and the first wire 712 is covered with a heat shrinkable sleeve 7122. The heat shrinkable sleeve 7122 provides support and insulation protection for the connecting point between the first wire 712 and the wiring part 711, so as to prevent the connecting point between the first wire 712 and the wiring part 711 from being broken. Meanwhile, it may also be dustproof and waterproof.

The wiring part 711 and the main body part 7101 of the electrode unit 710 together constitute the flexible circuit board 716 of the electrode sheet 71. In view of the formation of the electrode unit 710, the insulating plate 7102 is arranged on the side of the main body part 7101 of the flexible circuit board 716 away from the human skin, the dielectric element 7103 is arranged on the side of the main body part 7101 of the flexible circuit board 716 facing the human skin, and the temperature sensor 7104 is arranged on the side of the main body part 7101 of the flexible circuit board 716 facing the human skin.

The flexible circuit board 716 has an insulating substrate 7B and a plurality of conductive traces (not shown) embedded in the insulating substrate 7B. That is, the wiring part 711 and the main body part 7101 of the electrode unit 710 each includes the insulating substrate 7B and the plurality of conductive traces (not shown) embedded in the insulating substrate 7B. The plurality of conductive traces (not shown) in the insulating substrate 7B of the main body part 7101 are respectively electrically connected with the plurality of corresponding conductive traces (not shown) in the insulating substrate 7B of the wiring part 711. In the present embodiment, the flexible circuit board 716 has three paths of conductive traces (not shown), including one path of conductive trace (not shown) connecting all conductive cores 71051 of the conductive pad 7105 located on the main body part 7101 in series, one path of conductive trace (not shown) electrically connected with the ground terminal (not shown) of the temperature sensor 7104 located on the main body part 7101, and one path of conductive trace (not shown) electrically connected with the signal terminal (not shown) of the temperature sensor 7104 located on the main body part 7101. There are three golden fingers 7111 arranged on the side of the wiring part 711 facing the human skin. The three golden fingers 7111 of the wiring part 711 are electrically connected to the three conductive traces (not shown), respectively. The three golden fingers 7111 of the wiring part 711 are welded with an end of the first wire 712 away from the first plug 7121, so as to realize the electrical connection between the main body part 7101 of the electrode unit 710 and the first wire 712, and further realize the electrical connection between the dielectric element 7103 and the temperature sensor 7104 with the first wire 712 through the main body part 7101.

The conductive cores 71051 are exposed from the insulating substrate 7B of the main body party 7101. The insulating substrate 7B of the flexible circuit board 716 may isolate the water vapor in the air around the electrode sheet 71 from the solder (not shown) located between the conductive pad 7105 and the dielectric element 7103, and prevent the water vapor in the air away from the skin from eroding the solder (not shown) located between the dielectric element 7103 and the conductive pad 7105 on the main body part 7101 of the flexible circuit board 716. The insulating substrate 7B of the flexible circuit board 716 and the insulating plate 612 function as a double isolation, which may prolong the service life of the electrode sheet 71.

The backing 713 is in a sheet shape, which is mainly made of a flexible and permeable insulating material. The backing 713 is a mesh fabric. Specifically, the backing 713 is a mesh non-woven fabric, which has the characteristics of softness, lightness, moisture-proof and permeability; and can keep the patient's skin surface dry after being attached to the patient's body surface for a long time. The side of the backing 713 facing the patient's body surface is also coated with a biocompatible adhesive (not shown) for tightly adhering the backing 713 to the body surface corresponding to the patient's tumor site. In the present embodiment, only one electrode unit 710 is adhered to the backing 713. The backing 713 is substantially in a cubic sheet structure, and the four corners of the backing 713 are set in a rounded shape.

The support member 714 is adhered to the backing 713, and surrounds the outside of the electrode unit 710. In the middle of the support member 714, a through hole 7141 is disposed therethrough for accommodating the electrode unit 710. The support member 714 may be made of foam material. The support member 714 is flush with the surface of a side of the electrode unit 710 away from the backing 713. That is, the support member 714 is flush with the surface of a side of the electrode unit 710 facing the adhesive 715.

The adhesive 715 has double-sided adhesiveness. One surface of the adhesive 715 is adhered to the surface of the support member 714 and the surface of a side of the electrode unit 710 away from the backing 713. The other surface of the adhesive 715 is used as an adhesive lay, which is attached to the skin of human body surface to keep the skin surface moist and relieve local pressure. Preferably, the adhesive 715 may preferably be a conductive adhesive to serve as a conductive medium. With the support of the support member 714, the adhesive 715 has better adhesion to human skin.

Mainly referring to FIG. 24 to FIG. 25, the electrical connector 72 is provided with a plurality of sockets 721 which are plugged with the first plugs 7121 of the first wires 712 of the corresponding electrode sheets 71, and a second wire 722 which is plugged with an adapter (not shown) or an electric field generator (not shown). A second plug 7221 is arranged at an end of the second wire 722 away from the electrical connector 72, which may be directly connected with the electric field generator (not shown) or may be connected with the adapter (not shown) first and then connected with the electric field generator (not shown) through the adapter (not shown) to realize the electrical connection with the electric field generator (not shown). The plurality of sockets 721 and the second wire 722 are respectively arranged at opposite ends of the electrical connector 72. The electrical connector 72 is plugged with the first plugs 7121 of the first wires 712 of the electrode sheets 71 through the socket 721 thereof, so that the plurality of electrode sheets 71 are connected to the electrical connector 72 respectively to realize the electrical connection between the plurality of electrode sheets 71 and the electrical connector 72, and then realize the electrical connection between the plurality of electrode sheets 71 and the electric field generator (not shown) through the second plug 7221 plugged with the electric field generator (not shown) or the adapter (not shown). In use, the plurality of electrode sheets 71 are attached to the body surface corresponding to a patient's tumor site. The plurality of electrode sheets 71 is plugged into the corresponding sockets 721 of the electrical connector 72 through the first plugs 7121, and the electrical connector 72 is electrically connected with the electric field generator (not shown) through the second plug 7221, so as to realize the transmission of the alternating electric signals generated by the electric field generator (not shown) to the plurality of electrode sheets 71 through the electrical connector 72, and interfere or prevent the mitosis of the patient's tumor cells by applying the plurality of electrode sheets 71 to the patient's tumor site, so as to achieve the purpose of treating the tumor.

In the present embodiment, the plurality of electrode sheets 71 of the insulated electrode 700 are assembled on the electrical connector 72 in a detachable manner, and the plurality of electrode sheets 71 are connected to the electrical connector 72 in parallel, so that it may be easier to replace the damaged electrode sheets 71 when a certain one of the electrode sheets 71 is damaged and is unable to work, and there is no need to scrap all the plurality of electrode sheets 71, which may reduce the manufacturing cost and avoid waste, ensuring sufficient electric field strength during the tumor treating fields. Meanwhile, the plurality of electrode sheets 71 may be freely combined in quantity and freely adjusted in position according to the patient's body difference, tumor site and tumor size, ensuring the electric field strength applied to the patient's tumor site is optimal. In addition, the adhesion position of the plurality of electrode sheets 71 and the spacings between the plurality of electrode sheets 71 may also be freely adjusted according to the patient's own situation. It may allow the skin on the patient's tumor site to breathe freely, avoiding the rapid accumulation of heat on the patient's tumor site to which the electrode sheets 71 are adhered caused by a long-term treating field, and being not able to dissipate in time, which causes the patient's body surface where the electrode sheets 71 are adhered sweating and clogging pores and results in skin inflammation.

In the present embodiment, the number of the sockets 721 of the electrical connector 72 is nine, and the number of the electrode sheets 71 is nine. The electrical connector 72 is equipped with a body 720, and the body 720 is substantially in a polyhedral structure. In the present embodiment, the body 720 is substantially in a hexagonal prism structure. The nine sockets 721 are respectively arranged on multiple adjacent sides of the body 720, and an obtuse angle is formed between adjacent sides of the body 720. A second wire 722 is arranged on a side of the body 720 away from the sockets 721. In the present embodiment, the nine sockets 721 are evenly arranged on three adjacent sides of the body 720, and every three of the sockets 721 are arranged on a same side of the body 720 of the electrical connector 72. Terminals (not shown) inside the nine sockets 721 of the electrical connector 72 may be connected in series, so that the nine electrode sheets 71 are connected with each other in series. Terminals (not shown) inside the nine sockets 721 of the electrical connector 72 may also be connected in parallel, so that the nine electrode sheets 71 are connected with each other in parallel. When the terminals (not shown) inside the sockets 721 of the electrical connector 72 are connected in series, all the electrode sheets 71 need to be plugged with the electrical connector 72 for use.

When the terminals (not shown) inside the sockets 721 of the electrical connector 72 are connected in parallel, it may select some of the electrode sheets 71 to be plugged with the electrical connector 72 as needed, which will be more convenient and flexible for use. Alternatively, the terminals (not shown) inside the nine sockets 721 of the electrical connector 72 may be partially connected in series and partially connected in parallel. The terminals (not shown) inside the sockets 721 of the electrical connector 72 may be connected in series or parallel, or be partially connected in series and partially connected in parallel as needed, so that the plurality of electrode sheets 71 connected with the electrical connector 72 are all in series or all in parallel, or partially in series and partially in parallel. When the tumor is relatively large, it may select an appropriate number of electrode sheets 71 and freely adjust the spacings between the electrode sheets 71 as needed, so as to ensure the coverage area and the effect of treating fields of the insulated electrode 700 for tumor treating fields therapy. When the tumor is deviated to one side of the body, it may appropriately increase the number of the electrode sheets 71 of the insulated electrode 700 adhered to the other side of the body surface away from the tumor, so as to enhance the electric field strength on the side away from the tumor.

An alternate embodiment of the fourth embodiment of the insulated electrode

Referring to FIG. 29 and FIG. 30, the insulated electrode 700′ is an alternate implementation of the insulated electrode 700 of the above embodiment. The insulated electrode 700′ also includes: a plurality of electrode sheets 71′ for applying the alternating electric signals to a patient's tumor site; and an electrical connector 72′ electrically connected with an adapter (not shown) or an electric field generator (not shown). The plurality of electrode sheets 71′ are assembled on the electrical connector 72′ in a detachable plugging manner, so as to realize the electrical connection with the electrical connector 72′, and thus realize the electrical connection with the electric field generator (not shown) through the electrical connector 72′. Each electrode sheet 71′ includes: an electrode unit 710′, a wiring part 711′ connected to the electrode unit 710′, a first wire 712′ welded with the wiring part 711′, a backing 713′ adhered to the electrode unit 710′, a support member 714′ surrounding the electrode unit 710′ and adhered to the backing 713′, and an adhesive 715′ covering the corresponding part of the support member 714′ and the electrode unit 710′. The insulated electrode 700′ is different from the insulated electrode 700 of the previous embodiment in that: the insulated electrode 700′ includes three electrode sheets 71′, the body 720′ of the electrical connector 72′ is substantially in a triangular prism structure, the electrical connector 72′ is provided with three sockets 721′, and all the three sockets 721′ are arranged on the same side of the body 720′ of the electrical connector 72′. The wiring part 71l′ of each electrode sheet 71′ is connected with the corresponding first wire 712′ in a detachable plug-in manner. The wiring part 711′ of the electrode sheet 71′ is electrically connected with the first wire 712′ through a connector 7123′. The connector 7123′ includes a docking socket 7123A′ and a docking plug 7123B′. The docking socket 7123A′ is connected with the wiring part 711′, and the docking plug 7123B′ is connected with the end of the first wire 712′ away from the first plug 7121′. That is, the docking socket 7123A′ is arranged at the end of the wiring part 711′, and the docking plug 7123B′ is arranged at the end of the first wire 712′ away from the first plug 7121′. The docking socket 7123A′ and the electrode unit 710′ are respectively located at opposite ends of the wiring part 711′. The docking plug 7123B′ and the first plug 7121′ are respectively arranged at opposite ends of the first wire 712′. When the electrode unit 710′ of the electrode sheet 71′ is damaged and is unable to work, it may only replace the part of the electrode sheet 71′ except the first wire 712′, and the first wire 712′ can be continuously used, further reducing the cost of tumor treatment for patients.

The backing 713′ of the electrode sheet 71′ is substantially structured in a shape of the Chinese character “ [symbol] ”. The backing 713′ has two concave corners 7131′ recessed inward from two corners thereof. The two concave corners 7131′ are located at two corners of the backing 713′ away from the wiring part 711′. The concave corner 7131′ at the corner of the backing 713′ communicates with the outside and is in an “L” shape. An angle between two sides of the backing 713′ that forms the concave corner 7131′ is greater than or equal to 90 degrees, so that when the electrode sheet 71′ is attached to the body surface corresponding to the patient's tumor site, it may prevent the corners of the backing 713′ from arching and forming wrinkles, and further avoid air entering between the electrode unit 710′ and the skin from the wrinkles, which results in cryogenic burns due to the increase of heat generation of the electrode unit 710′ causing by the increase of the impedance between the electrode unit 710′ and the skin. The electrode unit 710′ is substantially in a square sheet structure. The main body part 7101′, the insulating plate 7102′, and the dielectric element 7103′ of the electrode unit 710′ are all in a square sheet structure. Two temperature sensors 7104′ are arranged on a side of the main body part 7101′ where the dielectric element 7103′ is arranged. The dielectric element 7103′ is provided with two perforations 71031′ for accommodating the temperature sensors 7104′ respectively. The two temperature sensors 7104′ are symmetrically arranged on the main body part 7101′, which can detect temperatures of human skin corresponding to different positions and ensure the accuracy of detection data. Four paths of conductive traces (not shown) are embedded in the insulating substrate 7B′ of the flexible circuit board 716′ which is mainly composed of the wiring part 711′ and the main body part 7101′ of the electrode unit 710′. The four paths of conductive traces (not shown) of the flexible circuit board 716′ are, respectively, one path of conductive trace (not shown) connecting all conductive cores (not shown) of a conductive pad (not shown) located in the main body part 7101′ in series: one path of conductive trace (not shown) connecting the ground terminals (not shown) of the two temperature sensors 7104′ located on the main body part 7101′ in series; and two paths of conductive traces (not shown) connecting the signal terminals (not shown) of the two temperature sensors 7104′ in parallel. There are four golden fingers (not shown) arranged on the side of the wiring part 711′ facing the human skin. The four conductive traces (not shown) are electrically connected to the four golden fingers (not shown) of the wiring part 711′ respectively.

At least one of the electrode sheet 71, 71′ of the insulated electrodes 700, 700′ of the present embodiment is detachably connected to an electric field generator (not shown) through the first wire 712, 712′ provided thereon, or may be first detachably connected to an adapter (not shown), and then electrically connected to the electric field generator (not shown) through the adapter (not shown), or is detachably connected to the electrical connector 72, 72′, and then electrically connected to the electric field generator (not shown) through the electrical connector 72, 72′, so as to realize the electrical connection with the electric field generator (not shown). And, each electrode sheet 71, 71′ only includes one electrode unit 710, 710′ electrically connected with the corresponding first wire 712, 712′. When the electrode unit 710, 710′ is damaged and is unable to work, it may only replace the corresponding electrode sheet 71, 71′, reducing the cost of tumor treatment for patients. In addition, the insulated electrodes 700, 700′ may freely combine the electrode sheets 71, 71′ in quantity or freely adjust electrode sheets 71, 71′ in positions according to a patient's tumor site, tumor position and tumor size, so as to ensure the coverage area for tumor treating fields therapy of the tumor treating fields system 3000, and ensure the electric field strength for tumor treating fields therapy of the tumor treating fields system 3000. At the same time, the relative spacing between the electrode sheets 71, 71′ allows the skin on the patient's body surface to breathe freely and exchange heat with the outside air, so as to avoid accumulation of heat on the patient's body surface caused by a long-term treating fields therapy, which causes sweating and clogging pores and results in skin inflammation.

A fifth embodiment of the insulated electrode

FIG. 31 to FIG. 36 illustrate the insulated electrode 5100 of the fifth embodiment of the present application, which includes: a backing 5002: an electrical functional component 5001 adhered to the backing 5002: a support member 5003 adhered to the backing 5002: an adhesive 5004 covering the corresponding parts of the support member 5003 and the electrical functional component 5001; and a wire 5006 welded with the electrical functional component 5001. The insulated electrode 5100 of the present embodiment is attached to the body surface corresponding to a patient's tumor site through the backing 5002, and an alternating electric signal is applied to the patient's tumor site through the electrical functional component 5001 to interfere or prevent the mitosis of the patient's tumor cells, so as to achieve the purpose of treating the tumor. The insulated electrode 5100 of the present embodiment is suitable for adhering to the trunk or head of a patient for tumor treating fields therapy, and a plurality of insulated electrodes 5100 may be freely combined.

The electrical functional component 5001 includes: a flexible circuit board 5011: an insulating plate 5012 and a dielectric element 5013 respectively arranged on opposite sides of the flexible circuit board 5011: a temperature sensor 5014 arranged on the flexible circuit board 5011 and located on the same side with the dielectric element 5013; and a reinforcement plate 5015 arranged on a side of the flexible circuit board 5011. The dielectric element 5013 and the temperature sensor 5014 are arranged on the side of the flexible circuit board 5011 close to the patient's body surface, and the insulating plate 5012 is arranged on the side of the flexible circuit board 5011 away from the patient's body surface. The electrical functional component 5001 is tightly attached to the backing 5002 by adhering the corresponding sites of the insulating plate 5012 and the flexible circuit board 5011 to the backing 5002, respectively.

The flexible circuit board 5011 includes: a main body part 5111; and a wiring part 5113 extended outward from the main body part 5111 and electrically connected to the wire 5006. In the present embodiment, the wiring part 5113 is in a strip shape or a band shape. The wiring part 5113 of the flexible circuit board 5011 is welded with the wire 5006 to realize the electrical connection between the wire 5006 and the electrical functional component 5001. The welding point between the wire 5006 and the wiring part 5113 is covered with a heat shrinkable sleeve 5061, which is used for sealing and providing insulation protection for the connecting point between the wire 5006 and the wiring part 5113 on the flexible circuit board 5011, and for improving the strength support, so as to prevent the connecting point between the wire 5006 and the electrical functional component 5001 from being broken. Meanwhile, it may also be dustproof and waterproof. One end of the wire 5006 is welded with the wiring part 5113 of the electrical functional component 5001, and the other end of the wire 5006 is provided with a first plug 5062 electrically connected to the adapter (not shown). Optionally, the first plug 5062 of the wire 5006 is directly electrically connected with the electric field generator 5200.

The main body part 5111 is in a circular sheet shape. The main body part 5111 is provided with a conductive pad 5114 corresponding to the dielectric element 5013. The conductive pad 5114 may be welded with the dielectric element 5013 through solder (not shown) to assemble the dielectric element 5013 on the main body part 5111 of the flexible circuit board 5011. The center of the conductive pad 5114 coincides with the center of the main body part 5111. The conductive pad 5114 has four conductive cores 5115 protruding or exposed from the main body part 5111. The conductive cores 5115 are center-symmetrically arranged, which may effectively prevent the position deviation of the dielectric element 5013 caused by the stacking of solder (not shown) during the welding process. The four conductive cores 5115 are arranged at intervals, which may reduce the consumption of copper foil for manufacturing the conductive cores 5115 and reduce the material cost. At the same time, it may save the amount of solder (not shown) used for welding the conductive cores 5115 and the dielectric element 5013, and further reduce the material cost. In other embodiments, the main body part 5111 may be in a shape of other polygonal sheets.

The main body part 5111 also has two pads 5117 protruding or exposed from the main body part 5111. The two pads 5117 are located approximately at the center of the area surrounded by the conductive pad 5114. The temperature sensor 5014 has a signal terminal (not shown) and a ground terminal (not shown). One of the pads 5117 on the main body part 5111 is welded with the signal terminal (not shown) of the temperature sensor 5014, and the other pad 5117 is welded with the ground terminal (not shown) of the temperature sensor 5014, so as to realize the electrical connection between the main body part 5111 and the temperature sensor 5014.

Both the main body part 5111 and the wiring part 5113 include an insulating substrate 5011A and a plurality of conductive traces (not shown) embedded in the insulating substrate 5011A. The plurality of conductive traces (not shown) in the insulating substrate 5011A of the main body part 5111 are electrically connected with the plurality of conductive traces (not shown) in the insulating substrate 5011A of the wiring part 5113, respectively, in one-to-one correspondence. That is, the flexible circuit board 5011 includes the insulating substrate 5011A and the plurality of conductive traces (not shown) embedded in the insulating substrate 5011A. In the present embodiment, both the main body part 5111 and the wiring part 5113 are arranged with three paths of the conductive traces (not shown). That is, the flexible circuit board 5011 is arranged with three paths of the conductive traces (not shown). The three paths of the conductive traces (not shown) include: one path of the conductive trace (not shown) connecting all conductive cores 5115 of the conductive pad 5114 located on the main body part 5111 in series: one path of the conductive trace (not shown) electrically connecting to one pad 5117 located on the main body part 5111 and welded with the ground terminal (not shown) of the temperature sensor 5014; and one path of the conductive trace (not shown) electrically connecting to the other pad 5117 located on the main body part 5111 and welded with the signal terminal (not shown) of the temperature sensor 5014.

A plurality of golden fingers 5116 respectively electrically connected with the three paths of the conductive traces (not shown) are arranged on a side of the wiring part 5113. The number of the golden fingers 5116 is consistent with the number of paths of the conductive traces (not shown). In the present embodiment, the number of the golden fingers is three. The three golden fingers 5116 are welded with the wire 5006, so that the wire 5006 is electrically connected to the three paths of conductive traces (not shown) of the flexible circuit board 5011, and then electrically connected to the dielectric element 5013 welded with the conductive pad 5114 and the temperature sensor 5014 welded with the pads 5117 through the three paths of conductive traces (not shown). Specifically, the wire 5006 has three signal wires (not shown), and the three signal wires (not shown) of the wire 5006 are respectively welded with the corresponding golden fingers 5116.

Referring to FIG. 35, the reinforcing plate 5015 is in a strip shape or a band shape. The reinforcing plate 5015 and the plurality of golden fingers 5116 of the wiring part 5113 are respectively arranged on opposite sides of the wiring part 5113. The reinforcing plate 5015 is located on the surface of a side of the wiring part 5113 away from the golden fingers 5116 and is arranged opposite to the golden fingers 5116, so that the pulling force of the wire 5006 on the wiring part 5113 is greatly dispersed during the process of moving or overturning the flexible circuit board 5011 after the wire 5006 is welded with the flexible circuit board 5011, and most of the pulling force is transferred to the reinforcing plate 5015, which is able to avoid the broken of the connection point between the golden fingers 5116 and the conductive traces (not shown) when the wire 5006 is pulled by the wiring part 5113. Preferably, the reinforcing plate 5015 is arranged on a side of the wiring part 5113 away from the golden fingers 5116, and is arranged opposite to the corresponding parts of the three golden fingers and the three paths of conductive traces (not shown). That is, the reinforcing plate 5015 is not only arranged opposite to the golden fingers 5116, but also arranged opposite to some conductive traces (not shown) connected to the golden fingers 5116. The area of the reinforcing plate 5015 is larger than the area of the corresponding golden fingers 5116. In the present embodiment, the area of the reinforcing plate 5015 is greater than 10 mm². The length of the reinforcing plate 5015 is not greater than the length of the wiring part 5113. The area of the reinforcing plate 5015 is not larger than the area of the wiring part 5113. Preferably, the wiring part 5113 is set with the same width as the reinforcing plate 5015. The length of the reinforcing plate 5015 is 5 mm to 40 mm. The reinforcing plate 5015 is made of a rigid reinforcing material with a thickness of 0.2 mm to 1 mm, such as epoxy glass fiber material and metal material. Preferably, the reinforcing plate 5015 is made of epoxy glass fiber material with a thickness of 0.2 mm to 0.5 mm. Optionally, the reinforcing plate 5015 is made of polyimide material with a thickness of 0.6 mm to 1 mm.

The insulating plate 5012 is in a circular sheet shape. The insulating plate 5012 is made of an insulating material, and is adhered to a side of the main body part 5011 of the flexible circuit board 5011 away from a patient's body surface, which may enhance the strength of the main body part 5011, and meanwhile provide a flat welding plane for the welding operation between the conductive pad 5114 and the dielectric element 5013 so as to improve the product yield rate. The insulating plate 5012 may isolate the water vapor in the air on the side of the electrical functional component 5001 away from the patient's body surface from entering the electrical functional component 5001, thereby preventing the water vapor from contacting the solder (not shown) located between the dielectric element 5013 and the main body part 5011, which may affect the electrical connection between the main body part 5011 and the dielectric element 5013.

The dielectric element 5013 is in a circular sheet shape. The dielectric element 5013 is made of a material with high dielectric constant, which may ensure human safety due to its characteristic of blocking the direct current and allowing the alternating current. The dielectric element 5013 has a dielectric constant of at least greater than 1000. An annular metal layer 5131 is attached to a side of the dielectric element 5013 facing the main body part 5111, which may be welded with the conductive pad 5114 on the main body part 5111 through a solder (not shown). The gap (not shown) formed by welding between the dielectric element 5013 and the main body part 5111 is filled with sealant (not shown) to protect the solder (not shown) between the dielectric element 5013 and the main body part 5111, so as to prevent the dielectric element 5013 from being affected by external force, which leads to the fracture of the welding part, and further leads to the failure of the alternating electric field to be applied to the patient's tumor site through the dielectric element 5013. At the same time, it can also prevent the water vapor in the air from entering the gap (not shown) and eroding the solder (not shown) between the dielectric element 5013 and the main body part 5111, which may affect the electrical connection between the dielectric element 5013 and the main body part 5111. The outer ring of the metal layer 5131 and the outer edge of the dielectric element 5013 are also arranged at intervals, so as to prevent the solder (not shown) between the metal layer 5131 of the dielectric element 5013 and the main body part 5111 from overflowing to the outside of the main body part 5111 when heated and melted, which may result in a direct current that is not hindered by the dielectric element 5013 passes through and acts on the patient's body surface when the insulated electrode 5100 is attached to the body surface of the patient's tumor site. The dielectric element 5013 has an opening 5132 disposed therethrough for accommodating the temperature sensor 5014. The edge of the opening 5132 of the dielectric element 5013 and the inner ring of the metal layer 5131 of the dielectric element 5013 are arranged at intervals, so as to prevent the solder (not shown) between the metal layer 5131 of the dielectric element 5013 and the main body part 5111 from diffusing toward the opening 5132 of the dielectric element 5013 when heated and melted, which may cause a short circuit of the temperature sensor 5014. The main body part 5111, the insulating plate 5012 and the dielectric element 5013 are arranged in one-to-one correspondence, and have centers located on the same straight line.

The temperature sensor 5014 is fixed at the center of the main body part 5111, and is used for monitoring the temperature of the adhesive 5004, so as to monitor the temperature of the human skin to which the adhesive 5004 is adhered. When the temperature monitored by the temperature sensor 5014 exceeds the upper limit of the safe temperature of human body, the electric field therapy instrument 1 shown in FIG. 1 or the electrical field generator (not shown) may reduce the alternating voltage of the alternating current of the alternating electrical signal or turn off the alternating electrical signal transmitted to the insulated electrode 5100 in time to avoid cryogenic burns of human body. The temperature sensor 5014 is welded to the two pads 5117 of the main body part 5111, and is then sealed with sealant (not shown) to prevent the water vapor from eroding the temperature sensor 5014 and causing a failure of the temperature sensor 5014. In the present embodiment, one temperature sensor 5014 is provided. In other embodiments, a plurality of temperature sensors 5014 may be disposed on the main body part 5111.

The support member 5003 is in a sheet shape. The supporting member 5003 is arranged around the dielectric element 5013 and adhered to the backing 5002. The support 5003 has a through hole 5031 disposed therethrough for accommodating the dielectric element 5013. The surface of the side of the support member 5003 close to the patient's body surface is flush with the surface of the side of the dielectric element 5013 close to the patient's body surface, so that the adhesive 5004 may be evenly covered on the support member 5003 and the dielectric element 5013, improving the comfort of applying the insulated electrode. In the present embodiment, the number of the support member 5003 is one.

The support member 5003 may be made of polyethylene (PE), or PET, or thermal conductive silica gel sheet, or a soft, chemically stable, light weight, non-deformable and non-toxic insulating material composed of polyurethane, polyethylene, dispersant, flame retardant and carbon fiber. Preferably; the support member 5003 may be made of flexible foam.

The adhesive 5004 is in a sheet shape. The adhesive 5004 has double-sided adhesiveness, with one side attached to the support member 5003 and the dielectric element 5013, and the other side attached to a patient's body surface. Preferably, the adhesive 5004 is made of a conductive hydrogel, serving as a conductive medium to conduct the alternating current passing through the dielectric element 5013 to the patient's tumor site. The number of the adhesive 5004 is the same as the number of the support member 5003. In the present embodiment, the number of the adhesive 5004 is one. The size of the adhesive 5004 is approximately the same as the size of the support member 5003. With the support of the support member 5003, the adhesive 5004 has better adhesion to human skin.

The flexible circuit board 5011 of the insulated electrode 5100 of the present embodiment has a reinforcing plate 5015 arranged on the wiring part 5113, located on the same side with the insulating plate 5012 and arranged opposite to the golden fingers 5116 of the wiring part 5113, which is to strengthen the strength of the connection part between the golden fingers 5116 of the wiring part 5113 and the conductive traces (not shown) thereof, so as to greatly disperse the pulling force of the wire 5006 on the wiring part 5113 during the process of the flexible circuit board 5011 being moved or turned over by the wire 5006, and prevent the connection point of the golden fingers 5116 on the wiring part 5113 and the conductive traces (not shown) from being broken when the wiring part 5113 is pulled by the wire 5006, which may otherwise cause the insulated electrode 5100 to become usable.

Referring to FIG. 37 to FIG. 43, the present application further provides a method of the tumor treating fields system applying alternating current signals.

FIG. 37 is a schematic block diagram of an embodiment of the electric field generator 8100 of the tumor treating fields system 3000 of the present application. As shown in FIG. 37, the electric field generator 8100 includes an alternating current signal generator 8110 and a signal controller 8120.

The alternating current signal generator 8110 is configured to generate at least two alternating current signals, and output the at least two alternating current signals to the corresponding at least two pairs of insulated electrodes, so as to generate alternating electric fields in at least two directions between the at least two pairs of the insulated electrodes.

The signal controller 8120 is configured to obtain temperature information detected and obtained by the insulated electrodes attached to the body surface corresponding to the tumor site, and individually control output of each of the at least two alternating current signals based on the temperature information, so as to selectively apply alternating current signals to the corresponding pairs of insulated electrodes to generate alternating electric fields in at least two directions between the insulated electrodes set in pairs.

In one example, the signal controller 8120 controls whether each alternating current signal generated by the alternating current signal generator 8110 to be output to the corresponding first pair of insulated electrodes 3001 or the second pair of insulated electrodes 3002. Each pair of insulated electrodes 3001, 3002 may include two of the afore-mentioned insulated electrodes 300, 400, 400′, 600, 600′, 700, 700′, and 5100. When the signal controller 8120 controls the first alternating current signal to be output to the corresponding first pair of insulated electrodes 3001, the first alternating current signal will generate an electric field 3003 in a first direction between the two insulated electrodes 3001. The two insulated electrodes 3001 can be attached to the body surface of the subject, so that the electric field 3003 in the first direction can be applied to the attached site. Similarly, when the signal controller 8120 controls the second alternating current signal, which is different from the first alternating current signal, generated by the alternating current signal generator 8110 to be output to the corresponding second pair of insulated electrodes 3002, the second alternating current signal will generate an electric field 3004 in a second direction between the two insulated electrodes 3002. Based on the temperature information of the first pair of insulated electrodes 3001 corresponding to the first alternating current signal and the temperature information of the second pair of insulated electrodes 3002 corresponding to the second alternating current signal, the signal controller 8120 may independently control whether the first alternating current signal and the second alternating current signal to be output to the corresponding first pair of insulated electrodes 3001 or the second pair of insulated electrodes 3002.

To sum up, the electric field generator 8100 may use the signal controller 8120 to control the various outputs of the alternating current signal generator 8110. Due to the separate control of the various alternating current signals, the controllability of applying electric fields to the corresponding insulated electrodes is improved.

FIG. 38 is a schematic block diagram of another embodiment of the electric field generator 8200 of the tumor treating fields system 3000 of the present application. As shown in FIG. 38, the electric field generator 8200 includes an alternating current signal generator 8210 and a signal controller 8220. The alternating current signal generator 8210 includes: a direct current signal source 8212 and a power converter 8214. The direct current signal source 8212 is configured to generate the direct current signal. In one example, a high-power direct current signal source may be used. The power converter 8214 is configured to convert the direct current signal into at least two alternating current signals.

In an exemplary embodiment, the alternating current signal generator 8210 further includes a direct current signal switch S1-8. The direct current signal switch S1-8 is electrically connected between the direct current signal source 8212 and the power converter 8214. The signal controller 8220 is configured to control a supply of the direct current signal from the direct current signal source 8212 to the power converter 8214 by controlling the direct current signal switch S1-8.

In an exemplary embodiment, the electric field generator 8200 further includes at least two pairs of output terminals. FIG. 38 illustrates the two pairs of output terminals (X1-8, X2-8) and (Y1-8, Y2-8). Each pair of output terminals is configured for supplying corresponding alternating current signal of the at least two alternating current signals from the alternating current signal generator 8210. In one example, the power converter 8214 converts the direct current signal of the direct current signal source 8212 into two alternating current signals with intermediate and high frequencies. The two alternating current signals are respectively defined as: a X-direction alternating current signal transmitted along an X-direction loop; and a Y-direction alternating current signal transmitted along a Y-direction loop, in which the pair of output terminals (X1-8, X2-8) constitutes the X-direction loop, and the pair of output terminals (Y1-8, Y2-8) constitutes the Y-direction loop. The X-direction alternating current signal generates an X-direction electric field between the corresponding first pair of insulated electrodes 3001, and the Y-direction alternating current signal generates a Y-direction electric field between the corresponding second pair of insulated electrodes 3002.

In an exemplary embodiment, the electric field generator 8200 further includes at least two pairs of switches S2-8, S3-8, S4-8 and S5-8. The at least two pairs of switches S2-8, S3-8, S4-8 and S5-8 are electrically connected to the at least two pairs of output terminals X1-8. X2-8. Y1-8 and Y2-8, respectively. The signal controller 8220 is configured to individually control output of the at least two alternating current signals from the at least two pairs of output terminals by individually controlling the at least two pairs of switches. FIG. 38 illustrates two pairs of switches S2-8, S3-8, S4-8 and S5-8. The pair of switches (S2-8, S3-8) is electrically connected to the pair of output terminals (X1-8. X2-8), and each of the switches is electrically connected to the corresponding output terminal, respectively. For example, the switch S2-8 is electrically connected to the output terminal X1-8 and the switch S3-8 is electrically connected to the output terminal X2-8. The pair of switches (S4-8, S5-8) and the pair of output terminals (Y1-8, Y2-8) are also electrically connected in a similar manner. Furthermore, the signal controller 8220 may control the output of X alternating current signal and Y alternating current signal from the pairs of output terminals (X1-8, X2-8) and (Y1-8, Y2-8) by individually controlling the pairs of switches (S2-8, S3-8) and (S4-8, S5-8). In various embodiments, the switches S1-8 to S5-8 may take any suitable form, such as electronic switches, mechanical switches, relays and the like.

In one example, when it is necessary to apply an X-direction electric field based on temperature information, the pair of switches (S2-8, S3-8) is closed. If it is not necessary to apply the X-direction electric field, the pair of switches (S2-8, S3-8) is opened, such that the pair of output terminals (X1-8, X2-8) cannot supply the X alternating current signal for establishing the X-direction electric field. For the Y-direction electric field, it can also be controlled based on temperature information in a similar way. It should be known that, the control of the X-direction electric field will not interfere with the control of the Y-direction electric field, and vice versa.

To sum up, the electric field generator 8200 may independently control the electric field applied to the corresponding body sites of the subject by individually controlling each pair of switches. For example, the electric field generator 8200 may independently control the X-direction and Y-direction electric fields, which improves the utilization rate of electric fields and ensures the therapeutic effect.

In an exemplary embodiment, signal controllers 8120, 8220, such as the signal controller 8120 in FIG. 37 or the signal controller 8220 in FIG. 38, are configured to monitor the obtained temperature information according to each of the above-mentioned insulated electrodes 3001, 3002, 300, 400, 400′, 600, 600′, 700, 700′, and 5100. In response to the temperature information being greater than a temperature threshold, it controls to stop outputting the alternating current signal applied to that pair of insulated electrodes; and in response to the temperature information being not greater than the temperature threshold, it controls to output the alternating current signal applied to that pair of insulated electrodes among the at least two alternating current signals. In one example, the temperature threshold may be set as 41° C., which is the upper limit of the safe temperature of the human body surface. Therefore, when the temperature information monitored by an insulated electrode of a pair of insulated electrodes is greater than 41° C., the signal controllers 8120, 8220 may control to stop outputting the alternating current signal applied to the pair of insulated electrodes containing that insulated electrode. At the same time, when the temperature information monitored by a pair of insulated electrodes is not greater than 41° C., the signal controllers 8120, 8220 may control to continuously output the alternating current signal applied to that pair of insulated electrodes. The range of the temperature threshold is 37° C.-41° C.

In the present application, the actions of “controlling to stop outputting the alternating current signals” and “controlling to output the alternating current signals” may be realized by controlling the opening and closing of the corresponding switches S2-8, S3-8, S4-8 and S5-8, respectively. However, it will be understood that, these actions do not necessarily require explicit physical operations. For example, if a switch was originally closed to output an alternating current signal, it is not necessary to perform any explicit physical operation to control that switch to output an alternating current signal, but only to maintain that switch in a closed state, such as by maintaining the supply of a control signal for closing the switch.

To sum up, the electric field generators 8100, 8200 of the present embodiment may independently control the output of alternating current signals based on the temperature information monitored by the insulated electrodes attached to the body surface of the subject through the signal controllers 8120, 8220, so as to ensure that the body temperature of the subject is within a safety threshold and avoid cryogenic burns.

FIG. 39 is a schematic block diagram of a tumor treating fields system 8300 according to an embodiment of the present application. The tumor treating fields system 8300 in the present embodiment includes at least two pairs of insulated electrodes 8320, 8330, 8340, 8350, and an electric field generator 8310. The insulated electrodes 8320, 8330, 8340, 8350 may be the insulated electrodes 300, 400, 400′, 600, 600′, 700, 700′, 5100 as described above in the first to the fifth embodiment. The at least two pairs of insulated electrodes 8320, 8330, 8340, 8350 are configured to be in contact with a corresponding body part of a subject. In one example, each of the insulated electrodes 8320, 8330, 8340, 8350 may include a plurality of capacitively coupled electrodes. The insulated electrodes may have good electrical contact with the body when placed on a subject's body. Each of the insulated electrodes 8320, 8330, 8340, 8350 has a temperature sensor array including temperature sensors 314, 414, 614, 714, 5014 as described above in the first to the fifth embodiment arranged on the insulated electrodes 8320, 8330, 8340, 8350.

The temperature sensor is configured to sense temperature signals of the adhesives 34, 44, 64, 715, 715′, 5004 attached to the corresponding body site to provide corresponding temperature information.

The electric field generator 8310 may be the electric field generator 8100 as shown in FIG. 37 or the electric field generator 8200 as shown in FIG. 38, or any electric field generator described in the embodiments.

In one example embodiment, the tumor treating fields system 8300 further includes an adapter 8360. The adapter 8360 is configured to convert the temperature signals from the temperature sensors of the insulated electrodes into temperature information, and transmit at least two alternating current signals to the corresponding at least two pairs of insulated electrodes. In one example, the temperature signals sensed by the temperature sensor arrays of at least two pairs of insulated electrodes are conducted into the adapter 8360 for processing, so as to obtain the temperature information that can be used by the signal controller 8120, 8220 of the electric field generator 8310. For example, the adapter 8360 may process the voltage values sensed by the temperature sensors into corresponding temperature values for the signal controller 8120, 8220 of the electric field generator 8310 to make a further determination.

To sum up, the tumor treating fields system 8300 for applying an electric field to a subject may collect temperature signals and feed them back to the electric field generator 8310, and the electric field generator 8310 controls the alternating current signal applied to the insulated electrodes based on the temperature information, thus ensuring the safety of the tumor treating fields system 8300 when applying electric fields. Because the electric field generator 8310 of the present embodiment can independently control the electric fields in all directions, it also ensures that the tumor treating fields system 8300 can apply electric fields purposefully.

FIG. 40 is a schematic flow diagram of the electric field generator 8310 of the tumor treating fields system 8300 applying an alternating current signal to the insulated electrodes, including Step 8410 and Step 8420. The electric field generator is the electric field generator 8100 shown in FIG. 37 or the electric field generator 8200 shown in FIG. 38.

Step 8410: Obtain temperature information of the insulated electrodes attached to the subject's body surface.

Step 8420: Based on the temperature information, individually controlling the output of each of the at least two alternating current signals, so as to selectively apply the alternating current signals to the insulated electrodes attached to the body surface corresponding to the tumor site and generate alternating electric fields in the at least two directions between the insulated electrodes.

FIG. 41 is a flowchart of controlling the electric field generator 8310 to apply alternating current signals to a pair of insulated electrodes in the Step 8420 shown in FIG. 40. The Step 8420 further includes the following Step 8510 to Step 8530.

Step 8510: Compare first temperature information with a temperature threshold. The first temperature information is temperature information corresponding to a temperature signal detected and obtained by the insulated electrodes that generates a first electric field of the alternating electric fields in the at least two directions.

Step 8520: In response to the first temperature information being greater than the temperature threshold, control to stop the output of a first alternating current signal of the at least two alternating current signals to the insulated electrodes that generates the first electric field.

Step 8530: In response to the first temperature information being not greater than the temperature threshold, control to continuously output the first alternating current signal to the insulated electrodes that generates the first electric field.

The range of the temperature threshold is 37° C.-41° C.

FIG. 42 is a further flowchart of controlling the electric field generator 8310 to apply alternating current signals to a pair of insulated electrodes in the Step 8420 shown in FIG. 40. The Step 8420 shown in FIG. 40 further includes the following Step 8610 to Step 8630.

Step 8610: Compare second temperature information with the temperature threshold.

The second temperature information is temperature information corresponding to a temperature signal detected and obtained by the insulated electrodes that generates a second electric field of the alternating electric fields in the at least two directions.

Step 8620: In response to the second temperature information greater than the temperature threshold, control to stop the output of a second alternating current signal of the at least two alternating current signals to the insulated electrodes that generates the second electric field.

Step 8630: In response to the second temperature information being not greater than the temperature threshold, control to continuously output the second alternating current signal to the insulated electrodes that generates the second electric field.

The range of the temperature threshold is 37° C.-41° C.

In an exemplary embodiment, the electric field generator 8310 continuously obtains the temperature information monitored and obtained by the insulated electrodes attached to the body surface of the tumor site, so as to control the output of the alternating current signal applied to the insulated electrodes in real time.

FIG. 43 is a flowchart of the operation of the tumor treating fields system 8300 of the present embodiment applying alternating current signals for tumor treatment. The method includes the following steps.

Step 8710: Turn on the tumor treating fields system 8300 to alternately apply alternating current signals to at least two pairs of insulated electrodes.

Step 8720: Continuously detect temperature signals, and feed back the temperature information corresponding to the temperature signals to the electric field generator.

Step 8730: Determine, by the electric field generator 8310, whether a first temperature information is greater than a temperature threshold. When the first temperature information is not greater than the temperature threshold, it proceeds to Step 8740. When the first temperature information is greater than the temperature threshold, it proceeds to Step 8750.

Step 8740: Continuously output, by the electric field generator 8310, a first alternating current signal to a first pair of insulated electrodes, so as to generate an electric field in a first direction between the first pair of insulated electrodes.

Step 8750: Control, by the electric field generator 8310, to stop outputting the first alternating current signal that generates the electric field in the first direction to the first pair of insulated electrodes, and apply a second alternating current signal to a second pair of insulated electrodes.

Step 8760: Determine, by the electric field generator 8310, whether a second temperature information is greater than the temperature threshold. When the second temperature information is not greater than the temperature threshold, it proceeds to Step 8770. When the second temperature information is greater than the temperature threshold, it proceeds to Step 8780.

Step 8770: Continuously output, by the electric field generator 8310, the second alternating current signal to the second pair of insulated electrodes, so as to generate an electric field in the second direction between the second pair of insulated electrodes.

Step 8780: Control, by the electric field generator 8310, to stop outputting the second alternating current signal that generates the electric field in the second direction to the second pair of insulated electrodes, and apply the first alternating current signal to the first pair of insulated electrodes.

The alternating current signals alternately applied in Step 8710 includes the first alternating current signal and the second alternating current signal. The first alternating current signal and the second alternating current signal are both sine wave signals, and have the same frequency and the same peak value of the AC voltage amplitude.

The temperature signal in Step 8720 is the temperature signal of the adhesive monitored and obtained by the temperature sensors of the insulated electrodes when applying alternating current signals to the insulated electrodes. The first temperature information in Step 8730 is obtained by the electric field generator 8310 or the adapter processing the feedbacked temperature signal of the first pair of insulated electrodes. The second temperature information in Step 8760 is obtained by the electric field generator 8310 or the adapter 8360 processing the feedbacked temperature signal of the second pair of insulated electrodes. The range of the temperature threshold in Step 8730 and Step 8760 is 37° C.-41° C. The electric field in the first direction in Step 8740 is perpendicular to the electric field in the second direction in Step 8770.

During the process of the tumor treating fields system 8300 applying alternating current signals through insulated electrodes, when any detected temperature information exceeds the temperature threshold, the electric field generator 8310 will turn off the alternating current signal applied to the pair of insulated electrodes until the temperature information of that pair of insulated electrodes returns to normal. However, turning off the output of the alternating current signal on one pair of insulated electrodes does not affect the output of the alternating current signal on the other pair of insulated electrodes. That is, when the temperature information on one pair of insulated electrodes exceeds the temperature threshold, the alternating current signal generated by the treating fields device 1 shown in FIG. 1 or the electrical field generator 8310 is switched to the other pair of insulated electrodes, which can ensure the continuous application of alternating current signals to the tumor site and ensure the therapeutic effect.

The present embodiment further provides a computer-readable storage medium having instructions stored thereon. When the instructions are executed by the signal controller 8120, 8220 of the electric field generator 8310 as mentioned above, the electric field generator 8310 is caused to perform the above-mentioned method.

The present embodiment further provides a computer program product comprising instructions. When the instructions are executed by the signal controller 8120, 8220 of the electric field generator 8310 as mentioned above, the electric field generator is caused to perform the above-mentioned method.

Referring to FIG. 44 to FIG. 52, the present application provides another embodiment of a tumor treating fields system 500 and a method for applying alternating current signals thereof. Referring to FIG. 44 to FIG. 49, the tumor treating fields system 500 of the present embodiment includes: an electric field generator 510 for generating alternating current signals: an adapter 520 electrically connected with the electric field generator 510; and two pairs of insulated electrodes 530 electrically connected with the adapter 520 and electrically connected with the electric field generator 510 through the adapter 520. The structure of the insulated electrode 530 of the present embodiment is similar to the structure of the insulated electrode 700 of the fourth embodiment of the present application, and the insulated electrode 530 also includes: an electrical connector 532 electrically connected with the adapter 520; and a plurality of electrode sheets 531 detachably assembled on the electrical connector 532. The insulated electrode 530 of the present embodiment may also be replaced by the insulated electrodes 700, 700′ of the fourth embodiment of the present application.

The structure of the electrode sheet 531 is completely the same as the structure of the electrode sheet 71 of the insulated electrode 700 in the fourth embodiment of the present application, and the electrode sheet 531 also includes: an electrode unit 533, a wiring part 534 electrically connected to the electrode unit 533, a first wire 535 welded with the wiring part 534, a backing 536 adhered to the electrode unit 533, a support member 537 adhered to the backing 536 in a manner of surrounding the electrode unit 533, and an adhesive 538 covering the corresponding part of the support member 537 and the electrode unit 533. One end of the first wire 535 is welded with the wiring part 534, and a heat shrinkable sleeve 5352 is provided at the welding point between the wiring part 534 and the first wire 535. The other end of the first wire 535 is detachably connected with the electrical connector 532 through a first plug 5351 provided at the end thereof. The electrode unit 533 further includes: a main body part (not labeled), arranged at the end of the wiring part 534 and electrically connected with the wiring part 534: an insulating plate 541 and a dielectric element 539 arranged at opposite sides of the main body part (not labeled), respectively; and a temperature sensor 540 arranged on the main body part (not labeled) and located at the same side as the dielectric elements 539. The specific structure of the electrode sheet 531 may refer to the description of the electrode sheet 71 in the fourth embodiment of the present application, and will not be repeated here. Of course, the electrode sheet 531 of the present embodiment may also be directly replaced by the insulated electrodes 300, 400, 400′, 600, 600′, 5100 in the first to the third, and the fifth embodiments of the present application. The electrode sheet 531 of the present embodiment includes a temperature sensor 540 for monitoring the temperature of the adhesive 538 applied to the body surface corresponding to a tumor site. The first wires 535 are all three-core cables, and each includes a core for transmitting alternating current signals, a core connected to the signal terminal TC1, TC2 . . . . TCn of the temperature sensor 540, and a core connected to the ground terminal GND of the temperature sensor 340.

The electrical connector 532 of the present embodiment is similar to the electrical connector 72 in the fourth embodiment of the present application in structure, and the electrical connector 532 also includes a plurality of first sockets 5321 which are plugged with the first plugs 5351 of the first wires 535 of the corresponding electrode sheets 531, and a second wire 5322 plugged with the adapter 520. A second plug 5324 is arranged at an end of the second wire 5322 away from the electrical connector 532, which may be directly plugged with the adapter 520 first, and then plugged with the electric field generator 510 through the adapter 520 to realize the electrical connection with the electric field generator 510. The plurality of first sockets 5321 and the second wire 5322 are respectively arranged at opposite ends of the electrical connector 532. The electrical connector 532 is plugged with the first plug 5351 of the first wire 535 of the electrode sheet 531 through the first socket 5321 thereof, so that the plurality of electrode sheets 531 are connected to the electrical connector 532 to realize the electrical connection between the plurality of electrode sheets 531 and the electrical connector 532, and then realize the electrical connection between the plurality of electrode sheets 531 and the adapter 520 through the second plug 5324 plugged with the adapter 520. In use, the plurality of electrode sheets 531 are attached to the body surface corresponding to a patient's tumor site. The plurality of electrode sheets 531 are plugged into the corresponding first sockets 5321 of the electrical connector 532 through the first plugs 5351, and the electrical connector 532 is plugged with the adapter 520 through the second plug 5324, so as to realize the transmission of the alternating electric signals generated by the electric field generator 510 to the plurality of electrode sheets 531 through the adapter 520 and the electrical connector 72, and interfere or prevent the mitosis of the patient's tumor cells by applying the plurality of electrode sheets 531 to the patient's tumor site, so as to achieve the purpose of treating the tumor.

In the present embodiment, the plurality of electrode sheets 531 of the insulated electrode 530 are assembled on the electrical connector 532 in a detachable manner, and the plurality of electrode sheets 531 are connected to the electrical connector 532 in parallel, so that it may be easier to replace the damaged electrode sheet 531 when a certain one of the electrode sheets 531 is damaged and is unable to work without needing to scrap all the plurality of electrode sheets 531, which may reduce the manufacturing cost and avoid waste, ensuring sufficient electric field strength during the tumor treating fields. Meanwhile, the plurality of electrode sheets 531 may be freely combined in quantity and freely adjusted in position according to the patient's body difference, tumor site and tumor size, ensuring the electric field strength applied to the patient's tumor site is optimal. In addition, the adhesion positions of the plurality of electrode sheets 531 and the spacings between the plurality of electrode sheets 531 may also be freely adjusted according to the patient's own situation. It may allow the skin on the patient's tumor site to breathe freely, avoiding the rapid accumulation of heat on the patient's tumor site to which the electrode sheets 531 are adhered caused by a long-term treating field, and being not able to dissipate in time, which causes the patient's body surface where the electrode sheets 531 are adhered sweating and clogging pores and results in skin inflammation.

In the present embodiment, the number of the first sockets 5321 of the electrical connector 532 is nine, and the number of the electrode sheets 531 is nine. The electrical connector 532 is provided with a body 5320, and the body 720 is substantially in a rectangular structure. The nine first sockets 5321 are all arranged on the same side of the body 5320, and the second wire 5322 is arranged on the side of the body 5320 away from the first sockets 5321. Terminals (not shown) inside the nine first sockets 5321 of the electrical connector 532 are connected in parallel, so that the nine electrode sheets 531 are connected with each other in parallel, so as to flexibly select the number of electrode sheets 531 plugged into the electrical connector 532 according to the actual situation such as tumor size, and freely adjust the interval between the electrode sheets 531, which will make it more convenient and flexible in use, and may ensure the coverage area and the treating effect of the insulated electrodes 500 for tumor treating fields therapy.

Inside the body 5320 of the electrical connector 532 of the present embodiment, there is also a switching circuit 5323 electrically connected with the first sockets 5321, which may independently control the turn-on and turn-off of the alternating current signals applied to the dielectric element 539 of the electrode sheet 531 after the corresponding electrode sheet 531 is plugged into the corresponding first socket 5321 through the first plug 5351 thereof, and May independently control the turn-on and turn-off of the transmission of the temperature signals detected and obtained by the corresponding electrode sheets 531. The first sockets 5321 of the electrical connector 532 are correspondingly plugged with the first plugs 5351 of the electrode sheets 531, thus forming a first connector 550 between the electrode sheets 531 and the electrical connector 532. The switching circuit 5323 includes a plurality of switches S1, S2. S3 . . . . Sn. The number of the switches S1, S2, S3 . . . . Sn is the same as the number of the electrode sheets E1, E2, E3 . . . . En plugged into the electrical connector 532. Switches S1, S2, S3 . . . . Sn of the switching circuit 5323 may be solid state relays or power triodes. States of the switches S1, S2, S3 . . . . Sn of the switching circuit 5323 are controlled by the signal processor 526 in the adapter 520. The signal processor 526 may independently control the turn-on and turn-off of each path of alternating current signals applied to the plurality of electrode sheets 531 through the switches S1, S2, S3 . . . . Sn, so as to realize the parallel transmission of multiple paths of alternating current signals.

Each of the electrode sheets E1, E2, E3 . . . . En has at least one temperature sensor T1, T2, T3 . . . . Tn, respectively. In the present embodiment, the switching circuit 5323 includes nine switches S1-S9 and nine electrode sheets 531, and each electrode sheet E is provided with a temperature sensor T. The nine switches are, respectively, used to independently control the turn-on and turn-off of alternating current signals applied to the corresponding electrode sheets E1-E9 and transmit the temperature signals of the corresponding temperature sensors T1-T9 to the adapter 520. The second wire 5322 is used to transmit the alternating current signals from the adapter 520 to the switching circuit 5323 of the corresponding insulated electrode 530, respectively, and may transmit the temperature signals detected and obtained by the corresponding insulated electrode 530 to the adapter 520. The tumor treating fields system 500 of the present embodiment may independently control the electrode sheets 531 electrically connected to the switching circuit 5323 through the switching circuit 5323 of the insulated electrode 530, so as to selectively apply alternating current signals to the corresponding electrode sheets 531. When the temperature signal detected by a certain electrode sheet 531 exceeds the temperature threshold set by the electric field generator 510, the tumor treating fields system 500 may independently disconnect the electrical connection between the electrode sheet 531 and the electrical connector 532 through the switching circuit 5323, so as to stop outputting alternating current signals to the electrode sheet 531, avoiding the electrode sheet 531 from continuously generating heat and heating up, which may result in cryogenic burns at where the electrode sheet 531 is attached. At the same time, the adapter 520 may continue to apply the alternating current signals generated by the electric field generator 510 to other electrode sheets 531, so as to continue the tumor treating fields therapy on the tumor site.

The second wire 5322 is a multi-core cable. For example, the second wire 5322 may be a 12-core copper cable, including one AC signal line for transmitting alternating current signals, nine temperature signal lines electrically connected one-on-one to the signal terminals of the respective temperature sensors 540 of the nine electrode sheets 531, one VCC line for supplying direct current to the switching circuit 5323, and one ground wire GND simultaneously connected to the ground terminals of all the temperature sensors 540 of the nine electrode sheets 531. In another embodiment, the second wire 5322 may be a 20-core copper cable, including nine cores respectively corresponding to the signal terminals TC1, TC2 . . . . TCn of the temperature sensors 540 of the nine electrode sheets 531, nine cores respectively corresponding one-on-one to the AC signal lines of the nine electrode sheets 531, one core corresponding to VCC for supplying direct current to the switching circuit 5323, and one core corresponding to the ground signal GND.

Mainly referring to FIG. 44, FIG. 47 and FIG. 48, the adapter 520 of the present embodiment is used to transmit the alternating current signals generated by the electric field generator 510 to the corresponding insulated electrode 530, and transmit back the temperature signals detected and obtained by the electrode sheets 531 of the corresponding insulated electrode 530 to the electric field generator 510, which includes a base 521 and a third wire 522 electrically connected to the base 521. The base 521 is internally provided with an analog-to-digital converter 525, a signal processor 526 communicatively connected with the analog-to-digital converter 525, a serial communication circuit 527 communicatively connected with the signal processor 526, and a buffer 528 communicatively connected with the analog-to-digital converter 525. The analog-to-digital converter 525 is configured to convert the received temperature of the corresponding electrode sheet 531 of the corresponding insulated electrode 530 into a digital signal, and transmit the converted digital signal to the signal processor 526 for processing. The analog-to-digital converter 525 may be an analog-to-digital conversion integrated circuit with a communication protocol (for example, SPI, I2C, etc.). The signal processor 526 is configured to calculate a corresponding temperature value based on the received digital signal from the analog-to-digital converter 525. The signal processor 526 may be an integrated circuit with data computation and storage capability (for example, a single chip microcomputer, FPGA, etc.). The serial communication circuit 527 is configured to serially transmit the received the temperature values from the signal processor 526 to the electric field generator 510. The serial communication circuit 527 may be an integrated circuit with a serial communication protocol (for example, RS232, RS485, etc.). As shown in FIG. 40 and FIG. 42, the analog-to-digital converter 525, the signal processor 526 and the serial communication circuit 527 of the adapter 520 are set independently. As shown in FIG. 41, the analog-to-digital converter 525 is built in the signal processor 526. The adapter 520 may also be configured with both the analog-to-digital converter 525 and the serial communication circuit 527 located in the signal processor 526 to simplify the circuit structure.

The base 521 is provided with four second sockets 523, which may be respectively plugged with the second plugs 5324 of the corresponding insulated electrodes 530 to realize the electrical connection between the adapter 520 and the corresponding insulated electrodes 530. The second sockets 523 and the third wire 522 are respectively arranged on two opposite sides of the base 521. The four second sockets 523 of the adapter 520 and the four second plugs 5324 of the insulated electrodes 530 are plugged in one-to-one correspondence to form a second connector 560 between the adapter 520 and the insulated electrodes 530. The end of the third wire 522 is provided with a third plug 524 which may be plugged with the electric field generator 510. The third wire 522 is an 8-core wire, in which four cores are AC lines X1, X2, Y1, Y2 that transmit alternating current signals to the four insulated electrodes 530; one core is a serial data transmission line TX: one core is a serial data reception line RX: one core is a VCC line that provides direct current to the adapter 520; and one core is a grounding signal line GND. The serial data transmission line TX is used to transmit temperature signals obtained by the temperature sensors 540 of the corresponding electrode sheets 531 to the electric field generator 510, and the serial data reception line RX is used to transmit control signals of the electric field generator 510 to the corresponding module.

The four second sockets 523 constitute the first connection ports X1, X2, Y1, Y2 where the adapter 520 is electrically connected with the four insulated electrodes 530. The two insulated electrodes 530 plugged into the first connection ports X1, X2 constitute a first pair of insulated electrodes, and the two insulated electrodes 530 plugged into the first connection ports Y1, Y2 constitute a second pair of insulated electrodes. Each of the first connector ports X1, X2. Y1, Y2 includes a power supply line VCC, a ground line GND, and an alternating current signal path line 570 composed of nine alternating current signals transmission lines. Each of the first connector ports X1, X2, Y1, Y2 further includes a temperature signal path line 580 composed of nine temperature signal transmission lines. The power supply voltage VCC, the alternating current signal path line 570 and the temperature signal path line 580 in the adapter 520 are all transferred to the corresponding insulated electrodes 530 through the first connector ports X1, X2, Y1, Y2. The nine temperature signal transmission lines in each insulated electrode 530 are reversely transmitted to the buffer 528 through the first connector ports X1, X2, Y1, Y2, and then transmitted to the analog-to-digital converter 525 to be converted into digital signals by the analog-to-digital converter 525, and then transmitted to the signal processor 526 for calculation. Finally, the signal processor 526 transmits the temperature value to the serial communication circuit 527 (for example, an integrated circuit with a serial communication protocol RS232), and the serial communication circuit 527 transmits the data to the electric field generator 510 through the third wire 527.

As shown in FIG. 49, the buffer 528 is configured to store temperature signals obtained by the temperature sensors 540 of the electrode sheets 531 and transmit the corresponding temperature signals to the analog-to-digital converter 525 for analog-to-digital conversion processing. The buffer 528 has a plurality of input terminals which are in one-to-one communication connection with the input terminals of the temperature sensors 540 of the electrode sheets 531 and a plurality of output terminals which are in one-to-one communication connection with the input terminals of the analog-to-digital converter 525. In an exemplary embodiment, the temperature signal lines TC1, TC2 . . . . TC3 of the temperature sensors T1, T2, T3 . . . . Tn of the electrode sheets E1, E2, E3 . . . . En may be connected to the plurality of input terminals of the buffer 528 in parallel through the second connector 560, and the ground terminals GND of the temperature sensors T1, T2, T3 . . . . Tn of the electrode sheets E1, E2, E3 . . . . En are cascaded together and then connected to the adapter 522. The buffer 528 may be composed of an operational amplifier circuit for isolating the preceding signal to protect the back-end analog-to-digital converter 525. The buffer 528 may also use a voltage follower circuit. The buffer 528 is electrically connected to the switching circuits 5323 of the insulated electrode 530 through the second connector 560.

Referring to FIG. 49, the adapter 520 further includes a voltage regulator VCC and a plurality of precision resistors R1-R9. The signal terminals of the plurality of temperature sensors (such as thermistor) T1-T9 are connected to the plurality of input terminals of the buffer 528 in one-to-one correspondence. The plurality of input terminals of the buffer 528 correspond to the plurality of output terminals thereof, respectively; and the plurality of output terminals of the buffer 528 are respectively connected to the plurality of precision resistors R1-R9 in one-to-one correspondence. The plurality of precision resistors R1-R9 are all connected in parallel to the voltage regulator VCC. That is, the plurality of precision resistors R1-R9 are respectively electrically connected between the voltage regulator VCC and the corresponding thermistor of the plurality of thermistors T1-T9 in one-to-one correspondence. For example, the precision resistor R1 is connected between the voltage regulator VCC and the thermistor T1. Since a change in temperature may synchronously cause a change in the resistance value of the thermistor, by connecting the precision resistor R and the voltage regulator VCC, the thermistor T and the precision resistor R are equivalent to two resistors connected in series for voltage division. The relationship between the resistance value R_{T} of the thermistor and the voltage V_{RT} satisfies:

$${V}_{R T} = VCC \times ( {R}_{T} / ( {R}_{T} + {R}_{S} ) )$$

Wherein, VCC is the supply voltage of the voltage regulator: R_{T} is the resistance value of the thermistor at temperature T (K); and R_{S} is the resistance value of the precision resistor connected with the thermistor. It can be seen that, when the resistance value R_{T} of the thermistor decreases due to the increase of temperature, the collected voltage V_{RT} also decreases. Since the voltage V_{RT} is an analog quantity, it is converted into a digital signal through the analog-to-digital converter 525. The signal processor 526 calculates the current temperature value based on the digital signal, where the relationship between the resistance value R_{T} of the thermistor and the voltage V_{RT} satisfies:

$${R}_{T} = {R}_{N} \times {e}^{B ( \frac{1}{T} - \frac{1}{{T}_{N}} )}$$

Wherein, R_{N} is the resistance value of the thermistor at the rated temperature T_{N}(K); T is the target temperature (K) in Kelvin, B is the thermal coefficient of the thermistor; and e is a constant (2.71828). For example, when a 3.3V power supply (VCC) and a thermistor with the thermal coefficient B of 3380 are used, R_{N} is 10K at 25° C. When the collected voltage V_{RT} is 1.5022V, the obtained R_{T} is about 8355.88 ohm. Meanwhile, the target T is calculated to be 29.8° C. In one example, the analog-to-digital converter 525 uses a 12-bit analog-to-digital conversion chip. Under a 3.3V power supply voltage, the minimum measured voltage is about 0.8056 mV, and the corresponding minimum temperature resolution is about 0.03° C. The accuracy of the measured temperature values is high. In addition, four groups of 36-channel thermistors T1, T2, etc, transmit voltage signals to the analog-to-digital converter 525 in parallel, and then the voltage signals are processed by the signal processor 526 and transmitted through the serial communication circuit 527, improving the transmission rate.

The electric field generator 510 is configured to: generate alternating current signals for tumor treating fields therapy; and transmit the alternating current signals to the switching circuit 5323 of the electrical connector 532 of the insulated electrode 530 plugged into the adapter 520 through the adapter 520 plugged into the electric field generator 510; and finally apply the alternating current signals to the corresponding electrode sheets 531 electrically connected with the switching circuit 5323 through the switching circuit 5323. Meanwhile, the electric field generator 510 is configured to receive the temperature signal from the temperature sensor 540 of the corresponding electrode sheet 531 of the corresponding insulated electrode 530 to regulate the alternating current signals applied to the electrode sheet 531.

Various electrode sheets 531 of the insulated electrode 530 of the tumor treating fields system 500 of the present embodiment are respectively connected to the electrical connector 532 in parallel, and the alternating current signals applied to each electrode sheet 531 is independently controlled by the switching circuit 5323 arranged in the electrical connector 532. The temperature signal detected and obtained by the temperature sensor 540 of the electrode sheet 531 may be transmitted to the adapter 520 through the switch Sn corresponding to the switching circuit 5323, and then the processed temperature signal may be transmitted to the electric field generator 510 through the adapter 520. The electric field generator 510 compares the temperature signal obtained through real-time monitoring with a preset temperature threshold, and regulates the alternating current signals applied to each electrode sheet 531 according to the comparison result or controls whether to apply the alternating current signals to the corresponding electrode sheet 531 through the switching circuit 5323. Therefore, it achieves the purpose of selectively applying alternating current signals to the corresponding electrode sheet 531, and controlling the heat generation of the site where the electrode sheet 531 is attached, avoiding cryogenic burns caused by the overhigh temperature of the body surface of the tumor site due to the heat of the electrode sheet 531.

The present embodiment further provides a method of the tumor treating fields system 500 applying alternating current signals. Referring to FIG. 50 to FIG. 53, the method includes the following steps.

Step S1: Receive, by the electric field generator 510, a plurality of temperature values, wherein the plurality of temperature values are numerical temperature values corresponding to temperature signals monitored and obtained by the temperature sensors 540 of the plurality of electrode sheets 531 of respective insulated electrodes 530.

Step S2: Control, by the electric field generator 510 based on the obtained plurality of temperature values, respective switching circuits 5323 of respective insulated electrodes 530 electrically connected to the electric field generator 510 to selectively transmit the corresponding alternating current signals to the corresponding electrode sheets 531 of the plurality of insulated electrodes 530.

Referring to FIG. 52, the step S2 of controlling, by the electric field generator 510 based on the obtained plurality of temperature values, respective switching circuits 5323 of respective insulated electrodes 530 electrically connected to the electric field generator 510 to selectively transmit the corresponding alternating current signals to the corresponding electrode sheets 531 of the plurality of insulated electrodes 530, further includes the following steps.

Step S20: Compare a plurality of first temperature values of the plurality of temperature values with a temperature threshold, wherein the first temperature value is a temperature value corresponding to a temperature signal monitored and obtained by the temperature sensor 540 of each electrode sheet 531 of a pair of insulated electrodes 530 that generates the electric field in the first direction.

Step S21: In response to one certain first temperature value being greater than the temperature threshold, the electric field generator 510 controls a corresponding switch Sn of the switching circuit 5323 of an insulated electrode 530 of one pair of insulated electrodes 530 that generate the electric field in the first direction to stop applying the alternating current signals to the electrode sheet 531 which obtains the temperature information corresponding to the first temperature value being greater than the temperature threshold and electrically connected with the switch Sn.

Step S22: In response to one certain first temperature value being not greater than the temperature threshold, the electric field generator 510 controls a corresponding switch Sn of the switching circuit 5323 of an insulated electrodes 530 of one pair of that generates the electric field in the first direction to continuously apply the alternating current signals to the electrode sheet 531 which obtains the temperature information corresponding to the first temperature value being not greater than the temperature threshold and electrically connected with the switch Sn.

In the Step S21, a range of the temperature threshold is set as 37° C.-41° C.

Referring to FIG. 52, the Step S2 further includes the following steps after the Step S22 is performed.

Step S31: Compare a plurality of second temperature values of the plurality of temperature values that are different from the first temperature values with the temperature threshold, wherein the second temperature value is a temperature value corresponding to a temperature signal monitored and obtained by the temperature sensor 540 of each electrode sheet 531 of the other pair of insulated electrodes 530 that generates the electric field in the second direction.

Step S32: In response to one certain second temperature value being greater than the temperature threshold, the electric field generator 510 controls a corresponding switch Sn of the switching circuit 5323 of the insulated electrode 530 of the other pair of insulated electrodes 530 that generates the electric field in the second direction to stop applying the alternating current signals to the electrode sheet 531 which obtains the temperature information corresponding to the second temperature value being greater than the temperature threshold and electrically connected with the switch Sn.

Step S33: In response to one certain second temperature value being not greater than the temperature threshold, the electric field generator 510 controls a corresponding switch Sn of the switching circuit 5323 of the insulated electrode 530 of the other pair of insulated electrodes 530 that generates the electric field in the second direction to continuously apply the alternating current signals to the electrode sheet 531 which obtains the temperature information corresponding to the second temperature value being not greater than the temperature threshold and electrically connected with the switch Sn.

In the Step S31, a range of the temperature threshold is set as 37° C.-41° C.

The tumor treating fields system 500 of the present embodiment may, based on the obtained temperature signals of respective electrode sheets 531 of the insulated electrodes 530, independently control the application of alternating current signals to each electrode sheet 531 electrically connected with the corresponding switch Sn of the switching circuit 5323 through the corresponding switch Sn of the switching circuit 5323 of the insulated electrode 530. When the temperature obtained by the electrode sheet 531 of a certain area exceeds the temperature threshold, it may turn off the switch Sn electrically connected with that electrode sheet 531 of that certain area, and stop the application of alternating current signals to the electrode sheet 531 in that certain area, so that the electrode sheet 531 of that certain area does not generate extra heat. After the temperature drops to a certain temperature being not greater than the temperature threshold, it may again turn on the switch Sn to continue to apply alternating current signals to the electrode sheet 531. The control of applying alternating current signals to each electrode sheet 531 does not interfere with each other, and the turn-off of a single path of alternating current signal has little influence on the electric field intensity generated by the whole alternating current signals, which optimizes the electric field intensity in unit area and avoids cryogenic burns at a position where the electrode sheet 531 is attached due to overhigh temperature.

The above of the present disclosure are only the preferred embodiments of the present disclosure, and are not intend to limit the present disclosure. Any modifications, equivalent alternatives, improvements, etc. made within the spirit and principles of the present disclosure shall be included within the scope of the protection of the present disclosure.

1. An insulated electrode for tumor treating fields therapy, comprising:
at least one electrode sheet configured to apply an alternating electric signal; and
an electrical connector detachably connected with the electrode sheet,
wherein the electrode sheet comprises: an individual electrode unit; and a first wire electrically connected with the electrode unit, wherein the electrode sheet is detachably connected with the electrical connector through the first wire.

2. The insulated electrode according to claim 1, wherein a plurality of electrode sheets are connected to the electrical connector in parallel through the corresponding first wires.

3. The insulated electrode according to claim 1- or 2, wherein the first wire of the electrode sheet has a first plug detachably plugged with the electrical connector, and wherein the first plug and the electrode unit are located at opposite ends of the first wire respectively.

4. The insulated electrode according to claim 3, wherein the electrical connector has a plurality of sockets which can be detachably plugged with the first plug of the first wire of the corresponding electrode sheet.

5. The insulated electrode according to claim 4, wherein the electrical connector is provided with a second wire, wherein the second wire and the plurality of sockets are respectively located at opposite ends of the electrical connector.

6. The insulated electrode according to claim 5, wherein the second wire has a second plug arranged at an end of the second wire.

7. The insulated electrode according to claim 6, wherein the electrical connector has a body, and wherein the plurality of sockets and the second wires are respectively arranged at opposite ends of the body.

8. The insulated electrode according to claim 3, wherein the electrode sheet further comprises: a wiring part connected with the electrode unit, and wherein the wiring part is welded with an end of the first wire away from the first plug.

9. The insulated electrode according to claim 8, wherein the electrode unit comprises: a main body part; and a dielectric element welded on one side of the main body part, and wherein the wiring part is laterally extended from the main body part.

10. The insulated electrode according to claim 9, wherein the wiring part and the main body part of the electrode unit form a flexible circuit board of the electrode sheet.

11. The insulated electrode according to claim 9, wherein the electrode unit further comprises: at least one temperature sensor, and wherein the temperature sensor is arranged on the main body part and at the same side as the dielectric element.

12. The insulated electrode according to claim 11, wherein at least one perforation is provided through the middle of the dielectric element, and wherein the temperature sensor is respectively accommodated in the corresponding perforation of the dielectric element.

13. The insulated electrode according to claim 9, wherein the electrode unit further comprises an insulating plate adhered to a side of the main body part away from the dielectric element.

14. The insulated electrode according to claim 8, wherein a periphery of a welding point of the first wire and the wiring part is covered with a heat shrinkable sleeve.

15. The insulated electrode according to claim 3, wherein the first wire is detachably connected with the electrode unit.

16. The insulated electrode according to claim 15, wherein the electrode sheet comprises: a wiring part electrically connected with the electrode unit, and wherein an end of the wiring part away from the electrode unit is configured with a docking socket.

17. The insulated electrode according to claim 16, wherein an end of the first wire away from the first plug is provided with a docking plug, and wherein the docking plug is detachably plugged with the docking socket.

18. The insulated electrode according to claim 1, wherein the electrode sheet further comprises: a backing adhered to the electrode unit; a support member arranged around the electrode unit and adhered to the backing; and an adhesive covering a side of the electrode unit and the support member away from the backing.

19. A tumor treating fields system, comprising:
an electric field generator; and
the insulated electrode according to claim 1, wherein the insulated electrode is connected with the electric field generator.

20. The tumor treating fields system according to claim 19, further comprising:
an adapter electrically connected with the electric field generator,
wherein the insulated electrode is detachably assembled on the adapter and is electrically connected with the electric field generator through the adapter.

21. The tumor treating fields system according to claim 19, wherein the insulated electrode is detachably assembled on the electric field generator.
